(19) United States
(12) Patent Application Publication
(10) Pub. No.: US 2025/0345412 A1
(43) Pub. Date: Nov. 13, 2025

(54) Coronavirus Vaccine

(71) Applicant: The Univesity of Melbourne Grattan Street, Victoria (AU)

(72) Inventors: Dale Ian Godfrey, Victoria (AU); David Charles Jackson, Victoria (AU); Nicholas Anthony Gherardin, Victoria (AU); Georgia Deliyannis, Victoria (AU); Damian Francis John Purcell

(21) Appl. No.: 18/269,108

(22) PCT Filed: Dec. 23, 2021
(86) PCT No.: PCT/AU2021/051553

§ 371 Date: Jun. 22, 2023

(30) Foreign Application Priority Data
Dec. 23, 2020 (AU) 2020904820
Dec. 2, 2021 (AU) 2021903911

(51) Int. Cl. A61K 39/215; A61K 39/00; A61P 37/04; C07K 14/005

(52) U.S. Cl. CPC A61K 39/215; A61P 37/04; C07K 14/005; A61K 2039/55511; C07K 2319/30; C12N 2770/20022; C12N 2770/20034

(57) ABSTRACT

The present invention relates to chimeric and fusion proteins and their compositions, and the use of such proteins and compositions in the prevention and/or treatment of coronavirus infections, or respiratory diseases or conditions associated with coronavirus infections.

### RELATED APPLICATION

This application claims the benefit of priority to Australian provisional application no. 2020904820 filed 23 Dec. 2020 and Australian provisional application no. 2021903911 filed 2 Dec. 2021, the entire contents of both are incorporated herein by reference.

### FIELD OF THE INVENTION

The present invention relates to chimeric and fusion proteins and their compositions, and the use of such proteins and compositions in the prevention and/or treatment of coronavirus infections, or respiratory diseases or conditions associated with coronavirus infections.

### BACKGROUND OF THE INVENTION

Emerging respiratory coronaviruses present a considerable threat to the health of global populations and the economy. Coronaviruses (CoVs) constitute a group of phylogenetically diverse enveloped viruses that encode large plus strand RNA genomes and replicate efficiently in most mammals. Human CoV (HCoVs-229E, OC43, NL63, and HKU1) infections typically result in mild to severe upper and lower respiratory tract disease.

Coronaviruses, belong to the Coronaviridae family in the Nidovirales order, are minute in size (80-200 nm in diameter) and contain a single-stranded RNA as a nucleic material, size ranging from 26 to 32 kbs in length. The subgroups of coronaviruses family are alpha (α), beta (β), gamma (γ) and delta (δ) coronavirus. The beta-coronaviruses are of the greatest clinical importance concerning humans. These include OC43 and HKU1 (which can cause the common cold) which are a beta-coronavirus of lineage A. Beta-coronaviruses of Lineage B include the severe acute respiratory syndrome coronaviruses SARS-COV and SARS-COV-2 (which causes the disease COVID-19). Middle East respiratory syndrome coronavirus (MERS-COV) is a beta-coronavirus from lineage C. These viruses cause acute lung injury (ALI) and acute respiratory distress syndrome (ARDS) which can lead to pulmonary failure and death.

Severe Acute Respiratory Syndrome-Coronavirus (SARS-COV) emerged in 2002-2003 causing ARDS with 10% mortality overall and up to 50% mortality in elderly populations.

Middle Eastern Respiratory Syndrome-Coronavirus (MERS-COV) emerged in the Middle East in April of 2012, manifesting as severe pneumonia, ARDS and acute renal failure.

In 2020, the world is faced with an extreme situation of a highly infectious coronavirus (2019-nCOV; SARS-COV-2) encountered by a global immunologically naïve population, manifesting as a disease termed “COVID-19”. SARS-COV-2 infections globally have exceeded 50 million confirmed cases with more than 1 million deaths to date, across more than 200 countries, areas or territories. COVID-19 manifestations range from mild to severe life-threatening with a substantial mortality rate.

There is a need for new or improved treatments for coronavirus infections and/or diseases associated with, or caused by, coronaviruses.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a chimeric or fusion protein for inducing an immune response to a coronavirus, the protein comprising 2 or more polypeptides comprising or consisting of an amino acid sequence of a receptor binding domain (RBD) from a Spike protein of a coronavirus linked to an Fc region of an antibody.

In another aspect, the present invention provides a chimeric or fusion protein for inducing an immune response to coronavirus, the protein comprising 2 or more polypeptides each comprising or consisting of an amino acid sequence of a receptor binding domain from a Spike protein of a coronavirus linked to a polypeptide comprising an Fc receptor binding domain.

In one aspect, the present invention provides a chimeric or fusion protein for inducing an immune response to coronavirus, the protein comprising a dimer of receptor binding domains from a Spike protein of a coronavirus linked to an Fc region of an antibody.

In another aspect, the present invention provides a chimeric or fusion protein for inducing an immune response to coronavirus, the protein comprising a dimer of receptor binding domains from a Spike protein of a coronavirus linked to a polypeptide comprising an Fc receptor binding domain. In one embodiment, the chimeric or fusion protein is a single chain dimer wherein a contiguous polypeptide chain comprises two RBD chain sequences that are covalently linked.

In any aspect, a receptor binding domain from a Spike protein of a coronavirus comprises, consists essentially of or consists of an amino acid sequence of any one or more of SEQ ID NO: 15 or 28, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NO: 15 or 28. In one embodiment, the amino acid sequence may include one or more of the mutations as shown in FIG. 6 or 7.

In any aspect, a receptor binding domain may be from a SARS-COV-2 variant. SARS-COV-2 variants have evolved from the original (Wuhan-Hu-1) ancestral or (“Wuhan”) strain of the virus (genome reference sequence: genbank accession NC_045512.2), herein referred to as wildtype (WT) strain. As described herein, a receptor binding domain may be from a WT (“Wuhan”), alpha, beta, gamma, kappa or delta strain or any other strain defined herein, including Table 3. In any embodiment, the receptor binding domain may be N334-P527 of a WT, alpha, beta, gamma, kappa or delta strain. The alpha variant carries 1 mutation in the RBD compared to the original WT strain: N501Y. The beta variant carries three mutations in the RBD compared to the original WT strain: N501Y, E484K and K417N. The gamma variant carries three mutations in the RBD compared to the original WT strain: K417T, E484K and N501Y. The kappa variant carries two mutations in the RBD compared to the original WT strain: L452R and E484Q. The delta variant carries two mutations in the RBD compared to the original WT strain: L452R and T478K.

In any aspect, a receptor binding domain from a Spike protein of a coronavirus comprises, consists essentially of or consists of an amino acid sequence of any one or more of SEQ ID NO: 15 or 28 having with 0 to 8 amino acid insertions, deletions, substitutions or additions (or a combination thereof). In some embodiments, the relevant amino acid sequence may have from 0 to 7, preferably from 0 to 6, preferably from 0 to 5, preferably from 0 to 4, preferably from 0 to 3, preferably from 0 to 2, preferably from 0 to 1 amino acid insertions, deletions, substitutions or additions (or a combination thereof), wherein the amino acid insertions, deletions, substitutions or additions (or a combination thereof) are located at the N- and/or C-terminus.

In any aspect, a receptor binding domain from a Spike protein of a coronavirus comprises, consists essentially of or consists of an amino acid sequence of any one or more of SEQ ID NO: 15 or 28 having with 0 to 8 amino acid insertions, deletions, substitutions or additions (or a combination thereof), and an Fc region of an antibody comprises, consists essentially of or consists of an amino acid sequence of any one of SEQ ID NO: 18 having with 0 to 8 amino acid insertions, deletions, substitutions or additions (or a combination thereof). In some embodiments, the relevant amino acid sequence may have from 0 to 7, preferably from 0 to 6, preferably from 0 to 5, preferably from 0 to 4, preferably from 0 to 3, preferably from 0 to 2, preferably from 0 to 1 amino acid insertions, deletions, substitutions or additions (or a combination thereof), wherein the amino acid insertions, deletions, substitutions or additions (or a combination thereof) are located at the N- and/or C-terminus.

In any aspect, the amino acid sequence of a chimeric or fusion protein of the invention comprises, consists essentially of or consists of a sequence as set forth in any one of SEQ ID NO: 2, 3, 5, 6, 22 or 23. In addition, the amino acid sequence of a chimeric or fusion protein of the invention comprises, consists essentially of or consists of N334-P527 of a receptor binding domain from a Spike protein of a SARS-COV-2 variant described herein, in particular a WT, alpha, beta, gamma, kappa or delta strain, linked directly or indirectly to a human IgG1 Fc, preferably the linker comprises or consists of an amino acid sequence of SEQ ID NO: 17 and the human IgG1 Fc comprises or consists of an amino acid sequence of SEQ ID NO: 18.

In any aspect, the Fc region of the antibody of the chimeric or fusion protein is an Fc region of an IgG, more preferably IgG1, more preferably a human IgG1. In some embodiments, the Fc region of an IgG is a mouse IgG1.

In any aspect, the one or more of the 2 or more polypeptides, or one or both of the receptor binding domains in the dimer of the chimeric or fusion protein, may be fused at the C-terminus to the Fc region. Alternatively, one or more of the 2 or more polypeptides, or one or both of the receptor binding domains in the dimer of the chimeric or fusion protein may be fused via a linker at the C-terminus to the Fc region.

Preferably, the Fc region of the chimeric or fusion protein comprises two heavy chain fragments, more preferably the CH2 and CH3 domains of said heavy chain. In one embodiment, the heavy chain fragments are linked via disulphide linkages.

In any aspect, an Fc region of an antibody comprises, consists essentially of or consists of an amino acid sequence of any one of SEQ ID NO: 16 or 18, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NO: 16 or 18.

In any aspect, an Fc region of an antibody comprises, consists essentially of or consists of an amino acid sequence of any one of SEQ ID NO: 16 or 18 having with 0 to 8 amino acid insertions, deletions, substitutions or additions (or a combination thereof). In some embodiments, the relevant amino acid sequence may have from 0 to 7, preferably from 0 to 6, preferably from 0 to 5, preferably from 0 to 4, preferably from 0 to 3, preferably from 0 to 2, preferably from 0 to 1 amino acid insertions, deletions, substitutions or additions (or a combination thereof).

In any aspect, a receptor binding domain from a Spike protein of a coronavirus comprises, consists essentially of or consists of an amino acid sequence of any one or more of SEQ ID NO: 15 or 28, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NO: 15 or 28, and an Fc region of an antibody comprises, consists essentially of or consists of an amino acid sequence of any one of SEQ ID NO: 16 or 18, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NO: 16 or 18. In one embodiment, the amino acid sequence of the receptor binding domain may include one or more of the mutations as shown in FIG. 6 or described herein in relation to a SARS-COV-2 variant, such as an alpha, beta, gamma, kappa, delta, delta plus, lambda, mu, iota or omicron strain.

A chimeric or fusion protein of the invention may be isolated, purified, substantially purified, enriched, synthetic or recombinant. In one embodiment, the purification is mediated by the use of a poly-His tag attached to the chimeric or fusion protein.

In another aspect of the invention, the present invention provides a composition comprising a chimeric or fusion protein of the invention as described herein and an adjuvant. Preferably, the composition further comprises a pharmaceutically acceptable carrier, diluent or excipient.

In any embodiment, the adjuvant is any one described herein, preferably a TLR2-agonist, more preferably a Pam-2-Cys containing molecule such as PEG-Four Arginine (R4)-Pam-2-Cys (PEG-R4-Pam-2-Cys), or preferably a stimulator of NKT cells, more preferably, any glycolipid that has the ability to stimulate NKT cells, such as alpha-Galactosylceramide (also referred to herein as “α-GalCer”), alpha-glucosylceramide, alpha-glucosyldiacylglycerol, alpha-galactosyldiacylglycerol, beta-mannosylceramide, and analogues thereof comprising variations in acyl and sphingosine chain lengths, saturation, and variations in polar head group composition.

In some embodiments, the TLR2-agonist can be selected from the group consisting of Pam-3-CSK4, PEG-R4-Pam-2-Cys, MALP-2, lipoteichoic acid, OspA, Porin, LcrV, lipomannan, Lysophosphatidylserine, Lipophosphoglycan (LPG), Glycophosphatidylinositol (GPI) and Zymosan.

In some embodiments, the adjuvant can be selected from the group consisting of poly-I: C, CpG, poly-ICLC, 1018 ISS, aluminum salts, Amplivax, AS03, AS04, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, Matrix M, MF59®, AddaVax™, monophosphoryl lipid A, Montanide IMS 1312, Montanide ISA 206, Montanide ISA V, Montanide ISA-51, OK-432, OM-174, OM-197-MP-EC, ONTAK. PEPTEL, vector system, PLGA microparticles, resiquimod, SRL172, Virosomes and other Virus-like particles, YF-17D, VEGF trap, R848, beta-glucan, Pam-3-Cys, and Aquila's QS21 stimulon.

In some embodiments, the adjuvant comprises a metabolizable oil and an emulsifying agent (such as a detergent or surfactant). Preferably, the oil and the emulsifying agent are present in the form of an oil-in-water emulsion having oil droplets substantially all of which are less than 1 micron in diameter. Exemplary metabolizable oils and emulsifying agents are described in U.S. Pat. Nos. 6,299,884 and 6,086,901. In one embodiment, the adjuvant comprises an oil-in-water emulsion. Preferably, the oil is squalene. Preferably, the aqueous phase is a citrate buffer (for example 10 mM at pH 6.5).

In one embodiment, the adjuvant comprises squalene in an oil-in-water emulsion. Preferably, the adjuvant further comprises TWEEN® 80 (polyoxyethylenesorbitan monooleate) and Span® 85 (sorbitan trioleate). The adjuvant may comprise 4.3% squalene, 0.5% TWEEN® 80 (polyoxyethylenesorbitan monooleate), 0.5% Span® 85 (sorbitan trioleate), optionally with 400 μg/ml MTP-PE (N-acetylmuramyl-L-alanyl-D-isoglutaminyl-L-alanine-2-[1,2-dipalmitoyl-sn-glycero-3-3(hydroxyphosphoryl-oxy)]ethylamide).

In one embodiment, the composition comprises 50% vol/vol adjuvant, preferably the adjuvant is MF59.

In one embodiment, the composition is lyophilised.

In another embodiment, the present invention provides a lyophilised composition comprising a chimeric or fusion protein of the invention as described herein and an adjuvant. Preferably the adjuvant is TLR2-agonist, more preferably a Pam-2-Cys containing molecule such as PEG-Four Arginine (R4)-Pam-2-Cys (PEG-R4-Pam-2-Cys).

The lyophilised composition may be stable for at least 1 month at temperatures ranging from −20° C. to 40° C. Preferably, the immunogenicity and protective efficacy of a reconstituted composition after storage in a lyophilised form for 1 month at temperatures ranging from −20° C. to 40° C. is at least 80%, 81%, 82%, 83%, 84%, 85%, 86%, 87%, 88%, 89%, 90%, 91%, 92%, 93%, 94%, 95%, 96%, 97%, 98%, 99% that of a reconstituted composition not subject to storage for 1 month at temperatures ranging from −20° C. to 40° C. Preferably, the antibody titres, preferably neutralising antibody titres, induced by a reconstituted composition after storage in a lyophilised form for 1 month at temperatures ranging from −20° C. to 40° C. is at least 80%, 81%, 82%, 83%, 84%, 85%, 86%, 87%, 88%, 89%, 90%, 91%, 92%, 93%, 94%, 95%, 96%, 97%, 98%, 99% that of a reconstituted composition not subject to storage for 1 month at temperatures ranging from −20° C. to 40° C. Preferably, the neutralising activity against multiple coronavirus variants, for example, the original (Wuhan-Hu-1) ancestral or (“Wuhan”) strain of SARS-COV-2 and the beta variant of SARS-COV-2 of a reconstituted composition after storage in a lyophilised form for 1 month at temperatures ranging from −20° C. to 40° C. is at least 80%, 81%, 82%, 83%, 84%, 85%, 86%, 87%, 88%, 89%, 90%, 91%, 92%, 93%, 94%, 95%, 96%, 97%, 98%, 99% that of a reconstituted composition not subject to storage for 1 month at temperatures ranging from −20° C. to 40° C.

In any aspect, the chimeric or fusion of the invention, or composition of the invention, induces an immune response to a coronavirus by inducing antibodies, preferably, the antibodies neutralise a coronavirus particle or virion. More preferably, the chimeric or fusion of the invention, or composition of the invention, induces immunoglobulins that specifically bind to a RBD of a coronavirus and inhibit the binding of the RBD to ACE2.

In any aspect of the invention, the chimeric or fusion protein, or composition of the invention, induces an immune response to a coronavirus by inducing a CD4 T cell response that promotes the production of antibodies by B cells.

In any aspect of the invention, the chimeric or fusion protein of the invention, or composition of the invention, induces an immune response to a coronavirus by inducing a CD8 T cell response that mediates cytotoxicity to coronavirus infected cells.

In any aspect of the invention, the chimeric or fusion protein of the invention, or composition of the invention, induces an immune response to a coronavirus by inducing an NKT cell response.

In any aspect of the invention, the chimeric or fusion protein of the invention, or composition of the invention, is administered as a single dose.

In any aspect of the invention, the chimeric or fusion protein of the invention, or composition of the invention, is administered as a prime-boost regime. The boost may be a first boost (i.e. second dose) or a second boost (i.e. third dose) or even a third or subsequent boost. The regimen may be a two-dose regimen or a three-dose regimen, or a four, five and beyond boost regimen for example, as described herein including the Examples. Preferably, the prime and boost doses are administered at different times. In some embodiments, the time interval between prime and boost is 1-week, 2-weeks, 3 weeks, 4-weeks, 6 weeks, 8-weeks, 3 months, 6 months, or 1 year.

In any aspect of the invention, the chimeric or fusion protein of the invention, or composition of the invention, is administered as two or more doses. In one embodiment, the chimeric or fusion protein of the invention, or composition of the invention, is administered as three separate doses.

In any aspect of the invention where prevention or prophylaxis is intended or required, the chimeric or fusion protein of the invention, or composition of the invention, is administered to the subject before any clinically or biochemically detectable symptoms of viral infection, preferably coronavirus infection. The subject may be identified as at risk of a coronavirus infection.

In any aspect of the invention, the chimeric or fusion protein of the invention, or composition of the invention, is administered to the respiratory tract. Typically, the chimeric or fusion of the invention, or composition of the invention, is administered to the upper and/or lower respiratory tract. For example, the chimeric or fusion of the invention, or composition of the invention, may be administered via inhalation or intranasally to the subject.

In any aspect of the invention, administration of chimeric or fusion protein of the invention, or composition of the invention, to a subject reduces viral load in the subject. Preferably, the viral load is reduced in the respiratory tract, for example, the upper and/or lower respiratory tract. More preferably, the viral load is reduced in the upper and lower respiratory tract. Preferably, the viral load is reduced in the lungs.

In any aspect, a composition of the invention may be formulated or adapted for administration to the respiratory tract, for example, the upper and/or lower respiratory tract. Preferably, the composition is formulated or adapted for inhalation or intranasal administration. In one embodiment, the composition is formulated as a spray, mist, or aerosol.

In a preferred embodiment, the composition is formulated as a nasal spray or as nasal drops.

In any aspect, the composition may be formulated or adapted for administration subcutaneously, intramuscularly or via any other route described herein.

In another aspect, the present invention accordingly further provides for a vaccine or immune stimulating composition for inducing an immune response to a coronavirus in a subject, the composition comprising:
an immunogen in the form of a chimeric or fusion protein as described herein, and
an adjuvant, for potentiating the immune response to the immunogen in the subject.

Preferably, the sole immunogen provided in the compositions, vaccines or immune stimulating compositions of the invention, is a chimeric or fusion protein as herein described.

In another aspect, the invention provides a pharmaceutical composition for treating and/or preventing (a) a disease associated with, or caused by, a coronavirus, or (b) a coronavirus infection, comprising as an active ingredient a chimeric or fusion protein of the invention and a pharmaceutically acceptable diluent, excipient or carrier. In one embodiment, the only active ingredient present in the composition is a chimeric or fusion protein of the invention.

In another aspect, the invention provides a pharmaceutical composition for treating and/or preventing (a) a disease associated with, or caused by, a coronavirus, or (b) a corona virus infection, comprising as a main ingredient a polypeptide or fusion protein of the invention and a pharmaceutically acceptable diluent, excipient or carrier. In one embodiment, the only active ingredient present in the composition is a chimeric or fusion protein of the invention.

In another aspect, the invention also provides a method of raising an immune response in a subject in need thereof, the method comprising administering to the subject a therapeutically effective amount of a chimeric or fusion protein of the invention, or a pharmaceutical composition of the invention, thereby raising an immune response in the subject.

The present invention also provides a method for inducing an immune response in a subject to a coronavirus, the method comprising administering to a subject in need thereof, a chimeric or fusion protein, vaccine or immune stimulating composition as described herein.

The present invention also provides a method of inducing a humoral immune response to a coronavirus in a subject, the method comprising administering to the subject, a chimeric or fusion protein, composition, vaccine or immune stimulating composition as herein defined. Preferably, the immune response that is induced comprises a “balanced” Th1-Th2 response.

The present invention also provides for methods of immunising a subject against a coronavirus infection, the method comprising administering to the subject, a chimeric or fusion protein, composition, vaccine or immune stimulating composition as herein defined.

The present invention provides a method for reducing or minimising the severity of a symptom associated with an infection with coronavirus, comprising administering to an individual in need thereof, a chimeric or fusion protein, composition, vaccine or immune stimulating composition as herein defined, wherein the symptoms are selected from the group consisting of fever, dry cough, tiredness, aches and pains, sore throat, diarrhoea, nausea and vomiting, conjunctivitis, headache, loss of taste or smell, a rash on skin, or discolouration of fingers or toes, difficulty breathing or shortness of breath, chest pain or pressure, and loss of speech or movement.

In another aspect, the invention also provides a method of treating and/or preventing (a) a disease associated with, or caused by, a coronavirus, or (b) a coronavirus infection in a subject in need thereof, the method comprising administering to the subject a therapeutically effective amount of a chimeric or fusion protein of the invention, or a composition of the invention, thereby treating and/or preventing (a) a disease associated with, or caused by, a coronavirus, or (b) a coronavirus infection in the subject.

In any aspect, a method of the invention further comprises a step of identifying a subject at risk of coronavirus infection.

In another aspect, the present invention provides for use of a chimeric or fusion protein of the invention, or a composition of the invention, in the preparation of a medicament treating and/or preventing (a) a disease associated with, or caused by, a coronavirus, or (b) a coronavirus infection in a subject in need thereof. Preferably, the medicament is adapted for administration to the upper respiratory tract.

In any embodiment, the invention also provides for use of the chimeric or fusion protein, or composition of the invention, for inhibiting or reducing the amount of coronavirus particles in the upper respiratory tract, or region thereof, or the lower respiratory tract, or region thereof, of an individual. A region of the upper or lower respiratory tract may be a region as described herein (e.g. the lung is a region of the lower respiratory tract). Preferably, the invention also provides for use of the chimeric or fusion protein, or composition of the invention, for inhibiting or reducing the amount of coronavirus particles in any tissue or organ where coronavirus is detected. Preferably, the chimeric or fusion protein of the invention, or composition of the invention, is adapted for use in the upper respiratory tract.

In any aspect, a chimeric or fusion protein of the invention is administered to the upper respiratory tract only. In other words, the chimeric or fusion protein of the invention, or composition of the invention, is not administered to the lower respiratory tract or to both the upper and lower respiratory tract (i.e. administered to the total respiratory tract).

In a further aspect, the invention provides a method of inhibiting or reducing the amount of coronavirus particles in the lung of an individual, the method comprising administering a chimeric or fusion protein, or composition of the invention to the upper respiratory tract of the individual, thereby inhibiting or reducing the amount of coronavirus particles in the lung of the individual.

In another aspect, the invention provides a method of inhibiting, delaying or reducing the progression of coronavirus particles from the upper respiratory tract to the lungs of an individual, the method comprising administering a chimeric or fusion protein of the invention, or composition of the invention, to the upper respiratory tract of the individual, thereby inhibiting, delaying or reducing the progression of the coronavirus particle from the upper respiratory tract to the lungs of the individual.

The invention further provides for use of chimeric or fusion protein of the invention in the preparation of a medicament for inhibiting, delaying or reducing the progression of coronavirus particles from the upper respiratory tract to the lungs of an individual. Preferably, the medicament is adapted for administration to the upper respiratory tract.

In any embodiment, the invention provides for use of a chimeric or fusion protein of the invention, or composition of the invention, for inhibiting, delaying or reducing the progression of coronavirus particles from the upper respiratory tract to the lungs of an individual. Preferably, the chimeric or fusion protein, or composition, of the invention is adapted for use in the upper respiratory tract.

In any method or use of the invention, the composition is a lyophilised composition and the method or use further comprises the step of reconstituting the lyophilised composition, and preferably administering that reconstituted composition to the individual.

Preferably, any method reduces or prevents dissemination of the coronavirus from the upper respiratory tract to the lungs. Preferably, the chimeric or fusion protein or composition of the invention is retained in the upper respiratory tract. In other words, the administration of the chimeric or fusion protein or composition of the invention to the upper respiratory tract prevents or reduces viral dissemination into the lungs.

As used herein, the upper respiratory tract may include any one or more of the following regions: the nose and nasal passages, nasal turbinates, paranasal sinuses, the pharynx, and the portion of the larynx above the vocal folds (cords).

Typically, the lower respiratory tract includes any one or more of the following regions: the portion of the larynx below the vocal folds, trachea, bronchi and bronchioles. The lungs can be included in the lower respiratory tract and include the respiratory bronchioles, alveolar ducts, alveolar sacs, and alveoli.

In any aspect of the present invention, administration to the upper respiratory tract may be administration to any one or more of the nose and nasal passages, paranasal sinuses, the pharynx, and the portion of the larynx above the vocal folds (cords). Consequently, a chimeric or fusion protein or composition of the invention may be (a) administered to the nose and nasal passages, (b) administered to the nose, nasal passages and paranasal sinuses, (c) administered to the nose, nasal passages, paranasal sinuses and the pharynx, or (d) administered to the nose, nasal passages, paranasal sinuses, the pharynx and the portion of the larynx above the vocal folds (cords).

In any aspect, a disease associated with, or caused by, a coronavirus is a respiratory disease.

In one aspect, the present invention provides for a chimeric or fusion protein of the invention, or a composition of the invention, for use in raising an immune response in a subject.

In one aspect, the present invention provides for a chimeric or fusion protein of the invention, or a composition of the invention, for use in treating and/or preventing (a) a disease associated with, or caused by, a coronavirus, or (b) a coronavirus infection in a subject in need thereof.

In any aspect of the present invention, the coronavirus infection may be an infection with a coronavirus from any of the genera Alphacoronavirus, Betacoronavirus, Gammacoronavirus or Deltacoronavirus. Preferably, the coronavirus is from one of the Alphacoronavirus subgroup clusters 1a and 1b or one of the Betacoronavirus subgroup clusters 2a, 2b, 2c, and 2d. The coronavirus may be any coronavirus that infects humans. Exemplary coronaviruses are SARS-COV, MERS-COV, SARS-COV-2, HCoV-NL63, HCOV-229E, HCoV-OC43 and HKU1, although the coronavirus may be any one as described herein. Most preferably, the coronavirus infection is an infection with SARS-COV-2.

In another aspect, the present invention also provides a method for obtaining an antibody directed to a coronavirus, the method comprising administering a chimeric or fusion protein, composition, vaccine or immune stimulating composition of the invention, to a non-human animal, thereby generating antibodies directed to a coronavirus in the animal. Preferably the method further comprises isolating the antibody from the animal.

In another aspect, the present invention also provides an antibody preparation comprising an antibody directed to a coronavirus, wherein the antibody preparation is obtained by administering a chimeric or fusion protein, composition, vaccine or immune stimulating composition of the invention, to a non-human animal, thereby generating antibodies directed to a coronavirus in the animal, and isolating the antibodies from the animal.

In another aspect, the invention also provides a nucleic acid molecule encoding a chimeric or fusion protein of the invention. The nucleic acid may be DNA or RNA (e.g. mRNA).

Preferably, the nucleic acid has a nucleotide sequence that encodes any one or more of the amino acid sequences corresponding to SEQ ID NO: 2, 5, 8, 12, 15, 22 or 23.

In any embodiment, the nucleic acid molecule encoding a chimeric or fusion protein comprises a nucleotide sequence corresponding to SEQ ID NO: 1, 4, 29, 30, 31 or 32.

In any embodiment, such a nucleic acid is included in an expression construct in which the nucleic acid is operably linked to a promoter. Such an expression construct can be in a vector, e.g., a plasmid.

In another aspect, the invention also provides a vector comprising a nucleic acid molecule of the invention.

In another aspect, the invention also provides a cell comprising a vector or nucleic acid of the invention. Examples of cells of the present invention include bacterial cells, yeast cells, insect cells or mammalian cells. Preferably, the cell is isolated, substantially purified or recombinant.

In another aspect, the invention also provides an animal or tissue derived therefrom comprising a cell of the invention.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Generation of variant RBD proteins. (A) Cartoon diagram of SARS-COV-2 virus covered in spike proteins at its surface and highlighting the receptor-binding domain (RBD) which binds ACE2 to elicit viral entry. (B) Cartoon diagram of different RBD proteins. (C-F) FPLC elution profiles from gel filtration size exclusion chromatography. Collected fractions are indicated. RBD monomer was eluted from a Superdex-75 column whereas RBD dimer and the RBD-Fc proteins were eluted from Superdex-200 column. (G) SDS-PAGE gel of the four RBD variants post gel-filtration. (H) Flow cytometry contour plots showing HEK293T cells transiently transfected to express human ACE2, or a T cell receptor (TCR) as a negative control, stained with RBD-Fc (mouse IgG1) or anti-TCR antibodies.

FIG. 2. RBD-specific antibody responses in primary and secondary sera from mice vaccinated with RBD-mouse IgG1-Fc dimer (RBD). BALB/c mice (n=5 per group) were vaccinated either via the subcutaneous route at the base of the tail or intranasally to the URT with 0.1 nmoles of RBD dimer administered without adjuvant (RBD) or administered in the presence of 0.3 nmoles PEG-R4-Pam-2-Cys (RBD P2C) or 0.2 μg a-GC (RBD a-GC). Mice were primed on day 0 and boosted on day 28. Sera collected just prior to the first injection (pre-bleed), just prior to the second injection (1°), and two weeks following the second injection (2°) were tested for RBD-specific antibodies by ELISA. Antibody titres were calculated as the reciprocal of the highest dilution of serum (log₁₀) giving an OD of 0.3. Antibody titres from individual animals are represented with the mean value for each group indicated by the horizontal line.

FIG. 3. SARS-COV-2 neutralising antibody titres in primary and secondary sera from mice vaccinated with RBD-mouse IgG1-Fc dimer (RBD). Sera from the BALB/c mice described in FIG. 2 were also assessed for SARS-COV-2 neutralising antibodies. Sera collected just prior to the second injection (19), and two weeks following the second injection (2°) were tested for neutralising antibodies using a microneutralization assay. Serial dilutions of individual serum samples were incubated with 100 TCID₅₀ of the SARS-COV-2 Wuhan Index strain VIC01 for 1 hour and residual virus infectivity was assessed using Vero cell monolayers set up in 96 well plates. Viral cytopathic effect was read on day 5. The neutralizing antibody titre was calculated using the Reed/Muench method (Reed, L. J. and Muench, H. (1938) A Simple Method of Estimating Fifty Percent Endpoints. American Journal of Hygiene, 27, 493-497). Neutralising antibody titres from individual animals are represented with the mean value for each group indicated by the horizontal line.

FIG. 4. Surrogate virus neutralisation test assessing neutralising activity of sera from mice vaccinated with RBD-mouse IgG1-Fc dimer (RBD). Secondary sera from the BALB/c mice described in FIG. 2 were also assessed for SARS-COV-2 neutralising antibodies using the surrogate virus neutralisation test. The capacity of serum antibodies to inhibit the binding of horseradish peroxidase conjugated SARS-COV-2 RBD protein (HRP-RBD) to wells coated with fixed ACE2 receptor was tested in an ELISA format at a serum dilution of 1:10. The degree to which serum inhibited binding of the HRP-RBD to ACE2 receptors, compared to control serum, was determined by optical density reading, with 20% inhibition and above considered a positive result. Percent inhibition of RBD binding to ACE-2 for individual animals are shown with the mean value for each group indicated by the horizontal line.

FIG. 5. Serological responses to SARS-COV-2 RBD in human convalescent patient samples (hCov) relative to serum samples from mice vaccinated with RBD-mouse IgG1-Fc dimer (RBD). RBD-specific antibody responses, and neutralising antibody responses assessed by a microneutralization assay and the surrogate virus neutralisation test were compared between sera from RBD vaccinated mice and plasma samples from hCov patients. The murine serum samples and hCov plasma samples were tested in separate experiments.

FIG. 6. Mutations in the SARS-COV-2 spike protein and neutralization of SARS-COV-2 variants by human convalescent plasma samples (hCov) and sera from mice vaccinated with RBD-mouse IgG1-Fc dimer (RBD). (A) Spike coding mutations and (frequency) with >10 local cases, mutations in the RBD region are shown in red. (B) SARS-COV-2 neutralisation assay showing inability or impaired ability of 3 convalescent patient plasma samples (DD2, DD3 and DD4) to neutralise 501Y and 477N mutants. (C) SARS-COV-2 neutralisation assay showing sera from mice immunized with RBD-mouse IgG1-Fc dimer vaccine, in the presence of P2C or a-GC adjuvant, neutralizing the 501Y and 477N mutants (VIC2089 and VIC4881) and the original VIC01 strain.

FIG. 7. Breadth of binding of antibodies in the sera of mice vaccinated with RBD-mouse IgG1-Fc dimer with or without adjuvant to RBD Variants. Using an RBD multiplex bead array assay, antibodies in sera collected from mice 4 weeks after a second subcutaneous dose of the RBD-mouse IgG1-Fc dimer vaccine administered in the presence of PEG-R4-Pam-2-Cys (RBD P2C) or a-GC (RBD a-GC) adjuvant, or without adjuvant (RBD) were tested for binding to 18 different RBD variants. Binding is indicated as the half-maximal effective concentration (EC₅₀) for each serum sample against each variant.

FIG. 8. SARS-COV-2 mouse challenge model established using VIC2089 (N501Y/D614G). BALB/c and C57BL/6 mice were aerosol challenged with either the Index SARS-COV-2 strain VIC01 or the VIC2089 (N501Y/D614G) variant. Three days after challenge mice were killed, right lung lobe harvested, homogenised and supernatants collected and stored at −80° C. Titre of infectious virus (TCID₅₀; 50% tissue culture infectious dose) was determined by titrating the supernatants on Vero cell monolayers and assessing CPE 5 days later.

FIG. 9. Comparing neutralisation of the Index SARS-COV-2 strain VIC01 (WT) and the VIC2089 (D614G/N501Y) variant by sera from mice vaccinated with RBD-mouse IgG1-Fc dimer. Sera collected 4 weeks following the second injection of RBD dimer administered without adjuvant (RBD) or administered in the presence of PEG-R4-Pam-2-Cys (RBD P2C) or a-GC (RBD a-GC) were tested for neutralising antibodies against VIC01 (WT) and the VIC2089 (D614G/N501Y) variant using a microneutralization assay. Viral CPE was read on day 5. The neutralizing antibody titre was calculated using the Reed/Muench method. Neutralising antibody titres from individual animals are represented with the mean value for each group indicated by the horizontal line.

FIG. 10. Lung viral titres in mice vaccinated with RBD-mouse IgG1-Fc dimer and challenged with VIC2089 (D614G/N501Y) variant. Mice immunised with the RBD-mouse IgG1-Fc vaccine alone (RBD), or with PEG-R4-Pam-2-Cys (RBD P2C) or a-GC (RBD a-GC) adjuvant were challenged with VIC2089 (D614G/N501Y) variant 103 days after the second dose of vaccine. Age matched unvaccinated control mice were also challenged at the same time. Three days after challenge mice were killed, right lung lobe harvested, homogenised and supernatants collected and stored at −80° C. Titre of infectious virus in the lungs (TCID₅₀) was determined by titrating the supernatants on Vero cell monolayers and assessing CPE 5 days later.

FIG. 11. Viral titres in nasal turbinates as a measure of upper airway protection of mice vaccinated with RBD-mouse IgG1-Fc dimer and challenged with VIC2089 (D614G/N501Y) variant. In addition to assessing lung viral titres in mice immunised with the RBD-mouse IgG1-Fc vaccine alone (RBD), or with PEG-R4-Pam-2-Cys (RBD P2C) or a-GC (RBD a-GC) adjuvant and challenged with VIC2089 (D614G/N501Y) variant, viral titres in the nasal turbinates of the same mice were also assessed as a measure of upper airway protection. Three days after challenge mice were killed, nasal turbinates harvested, homogenised and supernatants collected and stored at −80° C. Titre of infectious virus in the nasal turbinates (TCID₅₀) was determined by titrating the supernatants on Vero cell monolayers and assessing CPE 5 days later.

FIG. 12. Assessing protective efficacy of intramuscular administration of RBD-mouse IgG1-Fc (RBD) dimer vaccine by viral challenge. A group of 10 BALB/c mice were vaccinated intramuscularly with 0.1 nmoles of RBD dimer administered with 0.3 nmoles PEG-R4-Pam-2-Cys (RBD P2C). Mice were primed on day 0, boosted on day 28 and challenged four weeks later with VIC2089 (D614G/N501Y) variant. Five age matched unvaccinated control BALB/c mice were also challenged at the same time. Three days after challenge mice were killed, the right lung lobe and nasal turbinates were harvested, homogenised and supernatants collected and stored at −80° C. Titre of infectious virus in the tissues (TCID₅₀) was determined by titrating the supernatants on Vero cell monolayers and assessing CPE 5 days later.

FIG. 13. Assessing protective efficacy of intramuscular administration of RBD-mouse IgG1-Fc (RBD) dimer vaccine to C57BL/6 mice by viral challenge. A group of 5 C57BL/6 mice were vaccinated intramuscularly with 0.1 nmoles of RBD dimer administered with 0.3 nmoles PEG-R4-Pam-2-Cys (RBD P2C). Mice were primed on day 0, boosted on day 14 and challenged three months later with VIC2089 (D614G/N501Y) variant. Five age matched unvaccinated control C57BL/6 mice were also challenged at the same time. Three days after challenge mice were killed, the right lung lobe and nasal turbinates were harvested, homogenised and supernatants collected and stored at −80° C. Titre of infectious virus in the tissues (TCID₅₀) was determined by titrating the supernatants on Vero cell monolayers and assessing CPE 5 days later.

FIG. 14. Comparison of RBD-specific antibody responses in primary and secondary sera from mice vaccinated with RBD-mouse Fc dimer, RBD-human Fc dimer or RBD tandem dimer. (A) Schematic representation of the three RBD dimer antigens used for vaccination. (B) C57BL/6 mice (5 per group) were vaccinated intramuscularly with 0.1 nmoles of either RBD-mouse Fc dimer, RBD-human Fc dimer or single chain dimer all administered with 0.3 nmoles PEG-R4-Pam-2-Cys. Mice were primed on day 0 and boosted on day 28. Sera collected just prior to the first injection (pre-bleed), just prior to the second injection (1°), and two weeks following the second injection (2°) were tested for RBD-specific antibodies by ELISA. Antibody titres were calculated as the reciprocal of the highest dilution of serum (log₁₀) giving an OD of 0.3. Antibody titres from individual animals are represented with the mean value for each group indicated by the horizontal line.

FIG. 15. Durability of the RBD-specific antibody response elicited by each of the RBD antigens administered with PEG-R4-Pam-2-Cys as the adjuvant. RBD-specific antibody responses in the sera of C57BL/6 mice described in FIG. 14 were measured on days 42, 63 and 84 to assess the durability of the antibody response induced by the three different RBD antigens administered with PEG-R4-Pam-2-Cys (P2C). Antibody titres were calculated as the reciprocal of the highest dilution of serum (log₁₀) giving an OD of 0.3. Antibody titres from individual animals are represented with the mean value for each group indicated by the horizontal line.

FIG. 16. Comparison of RBD-specific antibody responses in sera of mice vaccinated with RBD-mouse IgG1-Fc dimer formulated with either MF59® or PEG-R4-Pam-2-Cys. C57BL/6 mice were immunised intramuscularly with 0.1 nmoles RBD mouse Fc dimer and 0.3 nmoles of PEG-R4-Pam-2-Cys, or 0.1 nmoles RBD mouse Fc dimer and MF59® (50% vol/vol). Mice were bled prior to the first immunisation (pre-bleeds), on day 14(1°) and boosted either on day 14 or day 28, and re-bled two weeks after the secondary immunisation in each case (2°). Total RBD-specific antibody titres were determined by ELISA. Antibody titres were calculated as the reciprocal of the highest dilution of serum (log₁₀) giving an OD of 0.3. Antibody titres from individual animals are represented with the mean value for each group indicated by the horizontal line.

FIG. 17. Comparison of RBD-specific antibody responses in sera of mice vaccinated with RBD-mouse IgG1-Fc dimer or RBD single chain dimer formulated with either MF59® or PEG-R4-Pam-2-Cys. C57BL/6 mice were immunised intramuscularly with 10 μg RBD mouse Fc dimer or 10 μg of RBD single chain dimer both formulated with either 0.3 nmoles of PEG-R4-Pam-2-Cys (P2C) or MF59® (50% vol/vol). Mice were primed on day 0 and boosted on day 14. Sera collected just prior to the first injection (pre-bleed), just prior to the second injection (1°), and four weeks following the second injection (2°) were tested for RBD-specific antibodies by ELISA. Antibody titres were calculated as the reciprocal of the highest dilution of serum (log₁₀) giving an OD of 0.3. Antibody titres from individual animals are represented with the mean value for each group indicated by the horizontal line.

FIG. 18. Comparison of RBD-specific antibody responses in sera of mice vaccinated with RBD monomer or RBD-mouse IgG1-Fc dimer formulated with PEG-R4-Pam-2-Cys. Groups of 10 BALB/c mice were immunised intramuscularly with either 0.2 nmoles RBD monomer formulated with 0.3 nmoles PEG-R4-Pam-2-Cys (P2C), or 0.1 nmoles RBD mouse Fc dimer formulated with 0.3 nmoles of PEG-R4-Pam-2-Cys (P2C). Mice were primed on day 0 and boosted on day 28. Sera collected just prior to the first injection (pre-bleed), just prior to the second injection (1°), and two weeks following the second injection (2°) were tested for RBD-specific antibodies by ELISA. Antibody titres were calculated as the reciprocal of the highest dilution of serum (log₁₀) giving an OD of 0.3. Antibody titres from individual animals are represented with the mean value for each group indicated by the horizontal line.

FIG. 19. Comparison of the protective efficacy induced by RBD monomer formulated with PEG-R4-Pam-2-Cys relative to RBD-mouse IgG1-Fc dimer formulated with PEG-R4-Pam-2-Cys. The groups of 10 BALB/c mice described in FIG. 18 were challenged four weeks after the day 28 boost with VIC2089 (D614G/N501Y) variant. Five age matched unvaccinated control BALB/c mice were also challenged at the same time. Three days after challenge mice were killed, the right lung lobe and nasal turbinates were harvested, homogenised and supernatants collected and stored at −80° C. Titre of infectious virus in the tissues (TCID₅₀) was determined by titrating the supernatants on Vero cell monolayers and assessing CPE 5 days later.

FIG. 20. Total and nAb responses in mice vaccinated intramuscularly with WT RBD-mFc and either PEG-R4-Pam-2-Cys or MF59®. (A-D) Total mouse Ig reactive to WT RBD monomer. (A and B) Total WT RBD antibody titres in primary (day 8, 15, 22, 41, 62 and 90) and secondary (day 128) sera of mice inoculated in 2-dose regime on days 0 and 112. ELISA titres are expressed as the reciprocal of the antibody dilution (log10) giving an absorbance of 0.3. This represents at least five times the background level of binding. (C and D) Total WT RBD antibody titres in primary (day 8, 15 & 22), secondary (day 41, 62, 90 & 111) and tertiary (day 132) sera from mice inoculated in 2-dose regime on days 0, 22 and 112. ELISA titres are expressed as the reciprocal of the antibody dilution (log10) giving an absorbance of 0.3. This represents at least five times the background level of binding. (E) Neutralising potency using WT Index strain (VIC01) via micro-neutralisation assay. Neutralisation titres of secondary (day 62 & 111) and tertiary (day 132) sera from mice vaccinated intramuscularly with WT RBD-mFc with MF59®. The half-maximal inhibitory concentration (IC50) was calculated based on the reciprocal dilution of serum that completely prevented CPE in 50% of the wells and was calculated by the Reed-Muench formula. LOD: Limit of detection

FIG. 21. Total and nAb responses in mice vaccinated intramuscularly with WT RBD-mFc+PEG-R4-Pam-2-Cys or WT RBD-nFc+PEG-R4-Pam-2-Cys. (A-B) Total mouse Ig reactive to WT RBD monomer. Total WT RBD antibody titres in primary (day 27) and secondary (day 41, 55, 76 and 115) sera from BALB/c mice (A) and C57BL/6 mice (B) vaccinated intramuscularly with WT RBD-mouse Fc or WT RBD-human Fc combined with PEG-R4-Pam-2-Cys. ELISA titres are expressed as the reciprocal of the antibody dilution (log10) giving an absorbance of 0.3. This represents at least five times the background level of binding. (C) Neutralising potency using WT Index strain (VIC01) via micro-neutralisation assay. Neutralisation titres of secondary (day 41) sera from C57BL/6 mice vaccinated intramuscularly with WT RBD-mouse Fc or WT RBD-human Fc combined with PEG-R4-Pam-2-Cys. The half-maximal inhibitory concentration (IC50) was calculated based on the reciprocal dilution of serum that completely prevented CPE in 50% of the wells and was calculated by the Reed-Muench formula.

FIG. 22. Viral titres in the lungs of C57BL/6 mice challenged with VIC2089 (N501Y/D614G). Mice vaccinated intramuscularly on days 0 and 28 with WT RBD-mouse Fc with PEG-R4-Pam-2-Cys, or WT RBD-human Fc with PEG-R4-Pam-2-Cys were aerosol challenged with VIC2089 on day 139 (111 days after the second immunisation). Age and sex matched unvaccinated control C57BL/6 mice were also challenged at the same time. Three days after challenge mice were killed, and the titre of infectious virus (TCID50) in the lungs of individual mice were determined by titrating lung supernatants on Vero cell monolayers and measuring viral CPE 5 days later.

FIG. 23. Total and nAb responses in mice vaccinated intramuscularly with WT RBD-mFc+MF59®. (A) Total WT RBD antibody titres in primary (1°, day 20) and secondary (2°, day 33) sera from mice vaccinated intramuscularly with 10 μg WT RBD-hFc formulated with MF59®. ELISA titres are expressed as the reciprocal of the antibody dilution (log10) giving an absorbance of 0.3. This represents at least five times the background level of binding. (B) Neutralisation of WT Index strain VIC01 or Beta variant B.1.351 via micro-neutralization assay. Neutralisation titres of secondary (day 45) sera from mice vaccinated intramuscularly with WT RBD-hFc with MF59®. The half-maximal inhibitory concentration (IC50) was calculated based on the reciprocal dilution of serum that completely prevented CPE in 50% of the wells and was calculated by the Reed-Muench formula.

FIG. 24. Viral titres in the lungs and nasal turbinates of mice challenged with VIC2089 (N501Y/D614G). Mice vaccinated intramuscularly on days 0 and 21 with 10 μg WT RBD-hFc formulated with MF59® were aerosol challenged with VIC2089 at 60 days after the second immunisation. Age and sex matched unvaccinated control mice were also challenged at the same time. Three days after challenge, mice were killed, and the titre of infectious virus (TCID50) in the lungs of individual mice were determined by titrating lung supernatants on Vero cell monolayers and measuring viral CPE 5 days later.

FIG. 25. Total and nAb responses in mice vaccinated intramuscularly with Beta RBD-mFc+MF59®. (A) Total WT antibody titres in primary (1°, day 20) and secondary (2°, day 33) sera from mice vaccinated intramuscularly with 10 μg Beta RBD-hFc or 3 μg Beta RBD-hFc formulated with MF59®. ELISA titres are expressed as the reciprocal of the antibody dilution (log10) giving an absorbance of 0.3. This represents at least five times the background level of binding. (B) Neutralisation of WT Index strain VIC01 or Beta variant B.1.351 via micro-neutralisation assay. Neutralisation titres of secondary (day 33) sera from mice vaccinated intramuscularly with 10 μg Beta RBD-hFc or 3 μg Beta RBD-hFc formulated with MF59®. The half-maximal inhibitory concentration (IC50) was calculated based on the reciprocal dilution of serum that completely prevented CPE in 50% of the wells and was calculated by the Reed-Muench formula.

FIG. 26. Total Ab responses in mice immunised with WT-RBD-hFC+MF59 or Beta RBD-hFc+MF59®. Total WT RBD antibody titres in primary sera (1°, day 20; A & C) and secondary sera (2°, day 37; B & D) from C57BL/6 mice vaccinated intramuscularly on days 0 and 21 with (10, 3, 1 or 0.3 μg) of WT RBD-hFc (A & B) or Beta RBD-hFc (C & D) in the presence of MF59®. ELISA titres are expressed as the reciprocal of the antibody dilution (log10) giving an absorbance of 0.3. This represents at least five times the background level of binding.

FIG. 27. Ab responses to WT RBD monomer and Beta RBD monomer in mice immunised with WT-RBD-hFC+MF59® or Beta RBD-hFc+MF59®. WT RBD-specific antibody titres (A) and Beta RBD-specific antibody titres (B) in secondary sera (day 56) from C57BL/6 mice vaccinated intramuscularly on days 0 and 21 with (10, 3, 1 or 0.3 μg) of WT RBD-hFc or Beta RBD-hFc in the presence of MF59®. ELISA titres are expressed as the reciprocal of the antibody dilution (log10) giving an absorbance of 0.3. This represents at least five times the background level of binding.

FIG. 28. Titre of virus in the lungs of C57BL/6 mice immunised with WT-RBD-hFC+MF59® or Beta RBD-hFc+MF59® and challenged with VIC2089 (N501Y/D614G) or the Beta variant B.1.351. Mice vaccinated intramuscularly on days 0 and 21 with 10, 3, 1 or 0.3 mg WT RBD-hFc or Beta RBD-hFc in the presence of MF59®, were aerosol challenged with VIC2089 (A), or Beta variant B.1.351 (B) 55 or 62 days respectively, after the second immunisation. Age and sex matched unvaccinated control C57BL/6 mice were also challenged on each of the corresponding days. Three days after challenge mice were killed, and the titre of infectious virus (TCID50; 50% tissue culture infectious dose) in the lungs of individual mice were determined by titrating lung and nasal supernatants on Vero cell monolayers and measuring viral cytopathic effect (CPE) 5 days later. LOD: Limit of detection.

FIG. 29. Anti-RBD antibody titres in hamsters. Anti-RBD antibody titres in hamsters, calculated as the reciprocal of the highest dilution of sera giving an O.D. value of 0.3. PBS control data for Day 14 was not included in the testing.

FIG. 30: CSU Hamster Study: Plaque reduction neutralization titres (PRNT) testing day 78 sera collected 14 days after the third dose against the WT strain.

FIG. 31: Viral titres in oropharyngeal swabs and lungs of hamsters immunised with WT RBD-hFc+MF59@ or Beta RBD-hFc+MF59® and challenged with WT (WA-01/USA) or Beta variant (B.1.351) virus. Hamsters were vaccinated intramuscularly on days 0, 21 and 64 with 30 or 10 μg of WT RBD-hFc, or 30, 10 or 3 μg of Beta RBD-hFc formulated with MF59® were challenged on day 85 under ketamine-xylazine anesthesia by intranasal instillation of 1×10⁴ pfu of virus in a volume of 100 μl. The SARS-COV-2 challenge strains used were WA-01/USA (WT) and Beta variant (B.1.351) and the hamsters were killed 3 days-post challenge. Viral load was determined in (A) oropharyngeal swabs collected 3 days post-challenge, just prior to the animals being euthanised and (B) cranial lung homogenates prepared on day 3 post-challenge. Viral titres were determined by performing serial ten-fold dilutions on confluent monolayers of Vero cells and counting plaques the following day. Titres are expressed as plaque forming units (pfu). LoD Limit of detection.

FIG. 32. Rat antibody titres specific for WT SARS-COV-2 spike antigen. IgG antibody titres to WT spike in sera from groups of 30 rats vaccinated intramuscularly on days 0, 22 and 43 with saline, 50 μg of Beta RBD-hFc or 50 μg of Beta RBD-hFc+MF59® adjuvant, as indicated at the base of each column. At termination of the toxicity on days 44-45, or day 56, a terminal blood sample was collected from each rat and processed to serum and assessed for SARS-COV-2 spike-specific IgG antibody responses by ELISA.

FIG. 33. Total mouse Ig reactive to WT RBD monomer. Total WT RBD antibody titers in (A) primary (day 21) and (B) secondary (day 43) sera from mice vaccinated intranasally with freshly prepared RBD-hFc+PEG-R4-Pam-2-Cys formulations or lyophilized RBD-hFc+PEG-R4-Pam-2-Cys formulations stored at different temperatures. ELISA titres are expressed as the reciprocal of the antibody dilution (log₁₀) giving an absorbance of 0.3. This represents at least five times the background level of binding.

FIG. 34. Neutralising potency. (A) Neutralisation of WT Index strain VIC01 via microneutralisation assay. Neutralisation titres of secondary (day 70) sera from mice vaccinated intranasally with freshly prepared RBD-hFc+PEG-R4-Pam-2-Cys formulations or lyophilized RBD-hFc+PEG-R4-Pam-2-Cys formulations stored at different temperatures. The half-maximal inhibitory dilution (ID50) was calculated based on the reciprocal dilution of serum that completely prevented CPE in 50% of the wells and was calculated by the Reed-Muench formula. (B) Percent Inhibition of RBD binding to ACE2 assessed using GenScript surrogate virus neutralization test (sVNT). sVNT titres of secondary (day 43) sera from mice vaccinated intranasally with freshly prepared RBD-hFc/PEG-R4-Pam-2-Cys formulations or lyophilized RBD-hFc/PEG-R4-Pam-2-Cys formulations stored at different temperatures.

FIG. 35. Titre of virus in the lungs and nasal turbinates of mice challenged with VIC2089 (N501Y/D614G). Mice vaccinated intranasally on days 0 and 30 with WT RBD-hFc+PEG-R4-Pam-2-Cys or lyophilized WT RBD-hFc+PEG-R4-Pam-2-Cys stored at 40° C. for one month, were aerosol challenged with VIC2089, 82 days after the second immunisation. Age and sex matched unvaccinated control mice were also challenged at the same time. Three days after challenge mice were killed, and the titre of infectious virus (TCID50) in the lungs (A) and nasal turbinates (B) of individual mice were determined by titrating lung and nasal supernatants on Vero cell monolayers and measuring viral CPE 5 days later.

FIG. 36. Neutralising potency. Neutralisation of WT Index strain VIC01 and Beta variant B1.351 via microneutralisation assay. Neutralisation titres of secondary (day 70) sera from mice vaccinated intranasally with freshly prepared WT RBD-hFc+PEG-R4-Pam-2-Cys or lyophilized WT RBD-hFc+PEG-R4-Pam-2-Cys stored at 40° C. for one month. The half-maximal inhibitory dilution (ID50) was calculated based on the reciprocal dilution of serum that completely prevented CPE in 50% of the wells and was calculated by the Reed-Muench formula.

### DESCRIPTION OF THE SEQUENCES

TABLE 1
Sequences of the invention

| SEQ ID | | |
|---|---|---|
| NO: | Description | Sequence |
| 1 | RBD-mouse IgG1 | gaattcaagcttgccaccatggggattcttcccagccctgggatgcctg |
| | Fc-fusion | cgctgctctccctcgtgagccttctctccgtgctgctgatgggttgcgtag |
| | nucleotide | ctgaaaccggtaacttgtgcccttttggtgaagtttttaacgccaccaga |
| | sequence | tttgcatctgtttatgcttggaacaggaagagaatcagcaactgtgttgct |
| | | gattattctgtcctatataattccgcatcattttccacttttaagtgttatgga |
| | | gtgtctcctactaaattaaatgatctctgctttactaatgtctatgcagattc |
| | | atttgtaattagaggtgatgaagtcagacaaatcgctccagggcaaa |
| | | ctggaaagattgctgattataattataaattaccagatgattttacaggct |
| | | gcgttatagcttggaactctaacaatcttgattctaaggttggtggtaatt |
| | | ataattacctgtatagattgtttaggaagtctaatctcaaaccttttgaga |
| | | gagatatttcaactgaaatctatcaggccggtagcacaccttgtaatggtg |
| | | ttgaaggttttaattgttactttcctttacaatcatatggtttccaacccac |
| | | taatggtgttggttaccaaccatacagagtagtagtactttcttttgaactt |
| | | ctacatgcaccagcaactgtttgtggacctggatccgggagcggctgc |
| | | aaaccctgtatctgcaccgtgcctgaagtctcctccgtctttatcttcccc |
| | | cccaagcctaaggacgtcctgaccatcaccctcacccctaaggtcac |
| | | ctgcgtcgtggtcgatatatccaaagatgaccccgaggtgcagttcag |
| | | ctggttcgtggacgatgtggaagtccacaccgcccagacccaaccc |
| | | agggaggagcagttcaactccaccttcaggtccgtgagcgagctgcc |
| | | cattatgcaccaggactggctcaacggaaaggaatttaagtgtagag |
| | | tcaacagcgctgccttccccgcccccatcgaaaagacaatctccaag |
| | | accaagggcaggcctaaagctcctcaggtctacaccatcccccccc |
| | | ccaaggagcagatggccaaggacaaggtgagcctcacctgcatga |
| | | tcacagacttcttccccgaggatataaccgtggagtggcagtggaatg |
| | | gccagcccgccgagaactataagaacacccagcccattatggaca |
| | | ccgacggcagctatttcgtctatagcaagctgaacgtccagaagtcca |
| | | attgggaagccggcaacacctttacctgctccgtgctgcatgagggcc |
| | | tgcacaatcaccacaccgagaagagcctgagccactcccccggca |
| | | agtgatgactcgag |
| 2 | RBD-mouse IgG1 | EFKLATMGILPSPGMPALLSLVSLLSVLLMGCVA [symbol] |
| | Fc-fusion amino | ETGNLCPFGEVFNATRFASVYAWNRKRISNCVAD |
| | acid sequence | YSVLYNSASFSTFKCYGVSPTKLNDLCFTNVYADS |
| | Translation of SEQ ID NO: 1 | FVIRGDEVRQIAPGQTGKIADYNYKLPDDFTGCVI |
| | pHLSec leader sequence | AWNSNNLDSKVGGNYNYLYRLFRKSNLKPFERDI |
| | shown in underline. | STEIYQAGSTPCNGVEGFNCYFPLQSYGFQPTNG |
| | Linker sequence shown in | VGYQPYRVVVLSFELLHAPATVCGPGSGSGCKPC |
| | bold. | ICTVPEVSSVFIFPPKPKDVLTITLTPKVTCVVVD |
| | 5′ cloning sites (ECOR1 and | SIKDDPEVQFSWFVDDVEVHTAQTQPREEQFNSTF |
| | HindIII) and Kozak | RSVSELPIMHQDWLNGKEFKCRVNSAAFPAPIEK |
| | sequences shown in italics. | TISKTKGRPKAPQVYTIPPPKEQMAKDKVSLTCMI |
| | \hat{ } refers to the predicted | TDFFPEDITVEWQWNGQPAENYKNTQPIMDTDG |
| | cleavage site based on | SYFVYSKLNVQKSNWEAGNTFTCSVLHEGLHNH |
| | SignalP5.0. | HTEKSLSHSPGK**LE |
| | * refers to stop codon | |
| 3 | RBD-mouse IgG1 | ETGNLCPFGEVFNATRFASVYAWNRKRISNCVAD |
| | Fc-fusion monomer | YSVLYNSASFSTFKCYGVSPTKLNDLCFTNVYADS |
| | amino acid | FVIRGDEVRQIAPGQTGKIADYNYKLPDDFTGCVI |
| | sequence | AWNSNNLDSKVGGNYNYLYRLFRKSNLKPFERDI |
| | Linker sequence shown in | STEIYQAGSTPCNGVEGFNCYFPLQSYGFQPTNG |
| | bold. | VGYQPYRVVVLSFELLHAPATVCGPGSGSGCKPCI |
| | | CTVPEVSSVFIFPPKPKDVLTITLTPKVTCVVVDI |
| | | SKDDPEVQFSWFVDDVEVHTAQTQPREEQFNSTF |
| | | RSVSELPIMHQDWLNGKEFKCRVNSAAFPAPIEK |
| | | TISKTKGRPKAPQVYTIPPPKEQMAKDKVSLTCMI |
| | | TDFFPEDITVEWQWNGQPAENYKNTQPIMDTDG |
| | | SYFVYSKLNVQKSNWEAGNTFTCSVLHEGLHNH |
| | | HTEKSLSHSPGK |
| 4 | RBD-human IgG1 | gaattcaagcttgccaccatggggatacttcccagccctgggatgcct |
| | Fc-fusion | gcgctgctctccctcgtgagccttctctccgtgctgctgatgggttgcgta |
| | nucleotide | gctgaagctagcaatctgtgccccttcggcgaggtgttcaacgccaca |
| | sequence | agattcgcctctgtgtacgcctggaaccggaagcggatcagcaattgc |
| | | gtggccgactacagcgtgctgtacaacagcgccagcttcagcacctt |
| | | caagtgctacggcgtgtcccctaccaagctgaacgacctgtgcttcac |
| | | caacgtgtacgccgacagcttcgtgatcagaggcgacgaagtgcgg |
| | | cagattgcccctggacagacaggcaagatcgccgattacaactaca |
| | | agctgcccgacgacttcaccggctgtgtgattgcctggaacagcaac |
| | | aacctggacagcaaagtcggcggcaactacaactacctgtaccggc |
| | | tgttccgcaagagcaacctgaagcctttcgagcgggacatcagcacc |
| | | gaaatctaccaggccggcagcaccccttgcaatggcgtggaaggctt |
| | | caactgctacttcccactgcaaagctacggcttccagcctacaaacgg |
| | | cgtgggctaccagccttacagagtggtggtgctgagcttcgagctgct |
| | | gcatgctcctgccacagtgtgtggacctggatccggcagcggtaccc |
| | | acacctgtcctccatgtcctgctccagaactgctcggcggaccttccgt |
| | | gttcctgtttcctccaaagcctaaggacaccctgatgatcagcagaac |
| | | ccctgaagtgacctgcgtggtggtggatgtgtctcacgaggaccccga |
| | | agtgaagttcaattggtacgtggacggcgtggaagtgcacaacgcca |
| | | agaccaagcctagagaggaacagtacaacagcacctacagagtg |
| | | gtgtccgtgctgaccgtgctgcaccaggattggctgaacggcaaaga |
| | | gtacaagtgcaaggtgtccaacaaggccctgcctgctcctatcgaga |
| | | aaaccatcagcaaggccaagggccagcctagggaaccccaggttt |
| | | acacactgcctccaagccgggaagagatgaccaagaaccaggtgt |
| | | ccctgacctgcctggtcaagggcttttaccccagcgacattgccgtgg |
| | | aatgggagagcaatggccagcctgagaacaactacaagaccaca |
| | | cctcctgtgctggacagcgacggctcattcttcctgtacagcaagctga |
| | | cagtggacaagagcagatggcagcagggcaacgtgttcagctgctc |
| | | cgtgatgcacgaggccctgcacaaccactacacccagaagtccctg |
| | | agcctgtctcctggcaagtgatgactcgag |
| 5 | RBD-human IgG1 | EFKLATMGILPSPGMPALLSLVSLLSVLLMGCVAE |
| | Fc-fusion amino | A [symbol] SNLCPFGEVFNATRFASVYAWNRKRISNCVAD |
| | acid sequence | YSVLYNSASFSTFKCYGVSPTKLNDLCFTNVYADS |
| | Translation of SEQ ID NO: 4 | FVIRGDEVRQIAPGQTGKIADYNYKLPDDFTGCVI |
| | pHLSec leader sequence | AWNSNNLDSKVGGNYNYLYRLFRKSNLKPFERDI |
| | shown in underline | STEIYQAGSTPCNGVEGFNCYFPLQSYGFQPTNG |
| | Linker sequence shown in | VGYQPYRVVVLSFELLHAPATVCGPGSGSGTHTC |
| | bold. | PPCPAPELLGGPSVFLFPPKPKDTLMISRTPEVTC |
| | 5′ cloning sites (ECOR1 and | VVVDVSHEDPEVKFNWYVDGVEVHNAKTKPREE |
| | HindIII) and Kozak | QYNSTYRVVSVLTVLHQDWLNGKEYKCKVSNKAL |
| | sequences shown in italics. | PAPIEKTISKAKGQPREPQVYTLPPSREEMTKNQV |
| | \hat{ } refers to the predicted | SLTCLVKGFYPSDIAVEWESNGQPENNYKTTPPV |
| | cleavage site based on | LDSDGSFFLYSKLTVDKSRWQQGNVFSCSVMHE |
| | SignalP5.0. | ALHNHYTQKSLSLSPGK**LE |
| | * refers to stop codon | |
| 6 | RBD-human IgG1 | EA\hat{ }SNLCPFGEVFNATRFASVYAWNRKRISNCVA |
| | Fc-fusion monomer | DYSVLYNSASFSTFKCYGVSPTKLNDLCFTNVYA |
| | amino acid | DSFVIRGDEVRQIAPGQTGKIADYNYKLPDDFTGC |
| | sequence showing | VIAWNSNNLDSKVGGNYNYLYRLFRKSNLKPFER |
| | cleavage site | DISTEIYQAGSTPCNGVEGENCYFPLQSYGFQPT |
| | Linker sequence shown in | NGVGYQPYRVVVLSFELLHAPATVCGPGSGSGT |
| | bold. | HTCPPCPAPELLGGPSVFLFPPKPKDTLMISRTPE |
| | \hat{ } refers to the predicted | VTCVVVDVSHEDPEVKFNWYVDGVEVHNAKTKP |
| | cleavage site based on | REEQYNSTYRVVSVLTVLHQDWLNGKEYKCKVS |
| | SignalP5.0. | NKALPAPIEKTISKAKGQPREPQVYTLPPSREEMT |
| | | KNQVSLTCLVKGFYPSDIAVEWESNGQPENNYKT |
| | | TPPVLDSDGSFFLYSKLTVDKSRWQQGNVFSCSV |
| | | MHEALHNHYTQKSLSLSPGK |
| 7 | RBD monomer | gaattcaagcttgccaccatggggatacttcccagccctgggatgcctg |
| | nucleotide | cgctgctctccctcgtgagccttctctccgtgctgctgatgggttgcgta |
| | sequence | gctgaagctagcaatctgtgccccttcggcgaggtgttcaacgccaca |
| | | agattcgcctctgtgtacgcctggaaccggaagcggatcagcaattgc |
| | | gtggccgactacagcgtgctgtacaacagcgccagcttcagcacctt |
| | | caagtgctacggcgtgtcccctaccaagctgaacgacctgtgcttcac |
| | | caacgtgtacgccgacagcttcgtgatcagaggcgacgaagtgcgg |
| | | cagattgcccctggacagacaggcaagatcgccgattacaactaca |
| | | agctgcccgacgacttcaccggctgtgtgattgcctggaacagcaac |
| | | aacctggacagcaaagtcggcggcaactacaactacctgtaccggc |
| | | tgttccgcaagagcaacctgaagcctttcgagcgggacatcagcacc |
| | | gaaatctaccaggccggcagcaccccttgcaatggcgtggaaggctt |
| | | caactgctacttcccactgcaaagctacggcttccagcctacaaacgg |
| | | cgtgggctaccagccttacagagtggtggtgctgagcttcgagctgct |
| | | gcatgctcctgccacagtgtgtggacctggatcccaccaccaccatca |
| | | ccactgatgactcgag |
| 8 | RBD monomer | EFKLATMGILPSPGMPALLSLVSLLSVLLMGCVAE |
| | amino acid | A [symbol] SNLCPFGEVFNATRFASVYAWNRKRISNCVAD |
| | sequence | YSVLYNSASFSTFKCYGVSPTKLNDLCFTNVYADS |
| | Translation of SEQ ID NO: 7 | FVIRGDEVRQIAPGQTGKIADYNYKLPDDFTGCVI |
| | pHLSec leader sequence | AWNSNNLDSKVGGNYNYLYRLFRKSNLKPFERDI |
| | shown in underline | STEIYQAGSTPCNGVEGFNCYFPLQSYGFQPTNG |
| | 6x His-Tag shown in bold. | VGYQPYRVVVLSFELLHAPATVCGPGSHHHHHH* |
| | 5′ cloning sites (ECOR1 and | *LE |
| | HindIII) and Kozak | |
| | sequences shown in italics. | |
| | \hat{ } refers to the predicted | |
| | cleavage site based on | |
| | SignalP5.0. | |
| | * refers to stop codon | |
| 9 | RBD monomer | EA\hat{ }SNLCPFGEVFNATRFASVYAWNRKRISNCVA |
| | amino acid | DYSVLYNSASFSTFKCYGVSPTKLNDLCFTNVYA |
| | sequence showing | DSFVIRGDEVRQIAPGQTGKIADYNYKLPDDFTGC |
| | cleavage site | VIAWNSNNLDSKVGGNYNYLYRLFRKSNLKPFER |
| | \hat{ } refers to the predicted | DISTEIYQAGSTPCNGVEGFNCYFPLQSYGFQPT |
| | cleavage site based on | NGVGYQPYRVVVLSFELLHAPATVCGPGSHHHH |
| | SignalP5.0. | HH |
| | 6x His-Tag shown in bold. | |
| 10 | RBD monomer | EA\hat{ }SNLCPFGEVFNATRFASVYAWNRKRISNCVA |
| | amino acid | DYSVLYNSASFSTFKCYGVSPTKLNDLCFTNVYA |
| | sequence without | DSFVIRGDEVRQIAPGQTGKIADYNYKLPDDFTGC |
| | His-tag showing | VIAWNSNNLDSKVGGNYNYLYRLFRKSNLKPFER |
| | cleavage site | DISTEIYQAGSTPCNGVEGFNCYFPLQSYGFQPT |
| | \hat{ } refers to the predicted | NGVGYQPYRVVVLSFELLHAPATVCGP |
| | cleavage site based | |
| | onSignalP5.0 | |
| 11 | RBD tandem dimer | gaattcaagcttgccaccatggggatacttcccagccctgggatgcct |
| | nucleotide | gcgctgctctccctcgtgagccttctctccgtgctgctgatgggttgc |
| | sequence | gtagctgaagctagcagagtgcagcccaccgagagcatcgtgcggttcc |
| | | ccaacatcaccaatctgtgccctttcggcgaggtgttcaacgccacca |
| | | gattcgcctctgtgtacgcctggaaccggaagcggatcagcaattgcg |
| | | tggccgactacagcgtgctgtacaacagcgccagcttcagcaccttc |
| | | aagtgctacggcgtgtcccctaccaagctgaacgacctgtgcttcacc |
| | | aacgtgtacgccgacagcttcgtgatcagaggcgacgaagtgcggc |
| | | agattgcccctggacagacaggcaagatcgccgattacaactacaa |
| | | gctgcccgacgacttcaccggctgtgtgattgcctggaacagcaaca |
| | | acctggacagcaaagtcggcggcaactacaactacctgtaccggct |
| | | gttccggaagtccaacctgaagcctttcgagcgggacatcagcaccg |
| | | agatctatcaggccggcagcaccccttgcaatggcgtggaaggcttc |
| | | aactgctacttcccactgcagtcctacggcttccagcctacaaacggc |
| | | gtgggctaccagccttacagagtggtggtgctgagcttcgagctgctg |
| | | catgctcctgccacagtgtgcggccctaagaaaagcaccaacctggt |
| | | caagaacaagcgggtgcagccaacagagtccattgtgcgctttccga |
| | | atatcaccaacctctgtccattcggggaagtctttaatgccacacgcttc |
| | | gccagcgtctacgcttggaataggaagagaatctccaactgcgtggcag |
| | | attactccgtcctgtacaattccgccagcttttccacgtttaagtgttat |
| | | ggcgtcagccccacaaagctcaatgatctctgctttaccaatgtctatg |
| | | ccgactcctttgtgattcgcggagatgaagtccgccagatcgctccag |
| | | gacagaccggaaaaatcgctgactataattacaagctcccggatgat |
| | | ttcacgggatgcgtgatcgcttggaactccaacaatctggattccaaa |
| | | gttggcgggaattacaattatctctatcgcctcttccgcaagagcaatct |
| | | gaagccctttgagagagacatctctacagaaatctaccaggctggctc |
| | | taccccatgcaacggcgtcgaggggtttaattgctatttcccgctgcaa |
| | | agctacgggtttcagcccaccaatggcgtcggatatcagccctatcgc |
| | | gtggtggtcctgtcttttgaactgctgcacgccccagctaccgtgtgtgg |
| | | cccaaaaaagtccaccaatctcgtgaagaacaaaggatcccacca |
| | | ccaccatcaccactgatgactcgag |
| 12 | RBD tandem dimer | EFKLATMGILPSPGMPALLSLVSLLSVLLMGCVAE |
| | amino acid | A [symbol] SRVQPTESIVRFPNITNLCPFGEVFNATRFASV |
| | sequence | YAWNRKRISNCVADYSVLYNSASFSTFKCYGVSPT |
| | Translation of SEQ ID NO: | KLNDLCFTNVYADSFVIRGDEVRQIAPGQTGKIAD |
| | 11 | YNYKLPDDFTGCVIAWNSNNLDSKVGGNYNYLYR |
| | pHLSec leader sequence | LFRKSNLKPFERDISTEIYQAGSTPCNGVEGENCY |
| | shown in underline | FPLQSYGFQPTNGVGYQPYRVVVLSFELLHAPAT |
| | 6x His-Tag shown in bold. | VCGPKKSTNLVKNKRVQPTESIVRFPNITNLCPFG |
| | 5′ cloning sites (ECOR1 and | EVFNATRFASVYAWNRKRISNCVADYSVLYNSAS |
| | HindIII) and Kozak | FSTFKCYGVSPTKLNDLCFTNVYADSFVIRGDEVR |
| | sequences shown in italics. | QIAPGQTGKIADYNYKLPDDFTGCVIAWNSNNLDS |
| | \hat{ } refers to the predicted | KVGGNYNYLYRLFRKSNLKPFERDISTEIYQAGST |
| | cleavage site based on | PCNGVEGFNCYFPLQSYGFQPTNGVGYQPYRVV |
| | SignalP5.0. | VLSFELLHAPATVCGPKKSTNLVKNKGSHHHHHH |
| | * refers to stop codon | **LE |
| 13 | RBD tandem dimer | EA\hat{ }SRVQPTESIVRFPNITNLCPFGEVFNATRFAS |
| | amino acid | VYAWNRKRISNCVADYSVLYNSASFSTFKCYGVSP |
| | sequence showing | TKLNDLCFTNVYADSFVIRGDEVRQIAPGQTGKIA |
| | cleavage site | DYNYKLPDDFTGCVIAWNSNNLDSKVGGNYNYLY |
| | \hat{ } refers to the predicted | RLFRKSNLKPFERDISTEIYQAGSTPCNGVEGFNC |
| | cleavage site based | YFPLQSYGFQPTNGVGYQPYRVVVLSFELLHAPA |
| | onSignalP5.0 | TVCGPKKSTNLVKNKRVQPTESIVRFPNITNLCPF |
| | 6x His-Tag shown in bold. | GEVFNATRFASVYAWNRKRISNCVADYSVLYNSA |
| | | SFSTFKCYGVSPTKLNDLCFTNVYADSFVIRGDEV |
| | | RQIAPGQTGKIADYNYKLPDDFTGCVIAWNSNNLD |
| | | SKVGGNYNYLYRLFRKSNLKPFERDISTEIYQAGS |
| | | TPCNGVEGFNCYFPLQSYGFQPTNGVGYQPYRV |
| | | VVLSFELLHAPATVCGPKKSTNLVKNKGSHHHHH |
| | | H |
| 14 | RBD tandem dimer | EA\hat{ }SRVQPTESIVRFPNITNLCPFGEVFNATRFASV |
| | amino acid | YAWNRKRISNCVADYSVLYNSASFSTFKCYGVSP |
| | sequence without | TKLNDLCFTNVYADSFVIRGDEVRQIAPGQTGKIA |
| | His-tag showing | DYNYKLPDDFTGCVIAWNSNNLDSKVGGNYNYLY |
| | cleavage site | RLFRKSNLKPFERDISTEIYQAGSTPCNGVEGFNC |
| | | YFPLQSYGFQPTNGVGYQPYRVVVLSFELLHAPA |
| | | TVCGPKKSTNLVKNKRVQPTESIVRFPNITNLCPF |
| | | GEVFNATRFASVYAWNRKRISNCVADYSVLYNSA |
| | | SFSTFKCYGVSPTKLNDLCFTNVYADSFVIRGDEV |
| | | RQIAPGQTGKIADYNYKLPDDFTGCVIAWNSNNLD |
| | | SKVGGNYNYLYRLFRKSNLKPFERDISTEIYQAGS |
| | | TPCNGVEGFNCYFPLQSYGFQPTNGVGYQPYRV |
| | | VVLSFELLHAPATVCGPKKSTNLVKNK |
| 15 | RBD (N334-P527) | NLCPFGEVFNATRFASVYAWNRKRISNCVADYSV |
| | amino acid | LYNSASFSTFKCYGVSPTKLNDLCFTNVYADSFVI |
| | sequence | RGDEVRQIAPGQTGKIADYNYKLPDDFTGCVIAW |
| | | NSNNLDSKVGGNYNYLYRLFRKSNLKPFERDISTE |
| | | IYQAGSTPCNGVEGFNCYFPLQSYGFQPTNGVGY |
| | | QPYRVVVLSFELLHAPATVCGP |
| 16 | Mouse IgG1 Fc | CKPCICTVPEVSSVFIFPPKPKDVLTITLTPKVTCVV |
| | amino acid | VDISKDDPEVQFSWFVDDVEVHTAQTQPREEQFN |
| | sequence | STFRSVSELPIMHQDWLNGKEFKCRVNSAAFPAPI |
| | | EKTISKTKGRPKAPQVYTIPPPKEQMAKDKVSLTC |
| | | MITDFFPEDITVEWQWNGQPAENYKNTQPIMDTD |
| | | GSYFVYSKLNVQKSNWEAGNTFTCSVLHEGLHN |
| | | HHTEKSLSHSPGK |
| 17 | Linker amino acid | GSGSG |
| | sequence | |
| 18 | Human IgG1 Fc | THTCPPCPAPELLGGPSVFLFPPKPKDTLMISRTP |
| | amino acid | EVTCVVVDVSHEDPEVKFNWYVDGVEVHNAKTK |
| | sequence | PREEQYNSTYRVVSVLTVLHQDWLNGKEYKCKV |
| | | SNKALPAPIEKTISKAKGQPREPQVYTLPPSREEM |
| | | TKNQVSLTCLVKGFYPSDIAVEWESNGQPENNYK |
| | | TTPPVLDSDGSFFLYSKLTVDKSRWQQGNVFSCS |
| | | VMHEALHNHYTQKSLSLSPGK |
| 19 | RBD-1 (R319- | RVQPTESIVRFPNITNLCPFGEVFNATRFASVYAW |
| | K537) amino acid | NRKRISNCVADYSVLYNSASFSTFKCYGVSPTKLN |
| | sequence | DLCFTNVYADSFVIRGDEVRQIAPGQTGKIADYNY |
| | | KLPDDFTGCVIAWNSNNLDSKVGGNYNYLYRLFR |
| | | KSNLKPFERDISTEIYQAGSTPCNGVEGFNCYFPL |
| | | QSYGFQPTNGVGYQPYRVVVLSFELLHAPATVCG |
| | | PKKSTNLVKNK |
| 20 | RBD-2 | RVQPTESIVRFPNITNLCPFGEVFNATRFASVYAW |
| | (R319-K537) | NRKRISNCVADYSVLYNSASFSTFKCYGVSPTKLN |
| | amino acid | DLCFTNVYADSFVIRGDEVRQIAPGQTGKIADYNY |
| | sequence | KLPDDFTGCVIAWNSNNLDSKVGGNYNYLYRLFR |
| | | KSNLKPFERDISTEIYQAGSTPCNGVEGFNCYFPL |
| | | QSYGFQPTNGVGYQPYRVVVLSFELLHAPATVCG |
| | | PKKSTNLVKNK |
| 21 | SARS-CoV-2 spike | MFVFLVLLPLVSSQCVNLTTRTQLPPAYTNSFTRG |
| | amino | VYYPDKVFRSSVLHSTQDLFLPFFSNVTWFHAIHV |
| | acid | SGTNGTKRFDNPVLPFNDGVYFASTEKSNIIRGWI |
| | sequence | FGTTLDSKTQSLLIVNNATNVVIKVCEFQFONDPFL |
| | | GVYYHKNNKSWMESEFRVYSSANNCTFEYVSQP |
| | | FLMDLEGKQGNFKNLREFVFKNIDGYFKIYSKHTPI |
| | | NLVRDLPQGFSALEPLVDLPIGINITRFQTLLALHR |
| | | SYLTPGDSSSGWTAGAAAYYVGYLQPRTFLLKYN |
| | | ENGTITDAVDCALDPLSETKCTLKSFTVEKGIYQTS |
| | | NFRVQPTESIVRFPNITNLCPFGEVFNATRFASVY |
| | | AWNRKRISNCVADYSVLYNSASFSTFKCYGVSPT |
| | | KLNDLCFTNVYADSFVIRGDEVRQIAPGQTGKIAD |
| | | YNYKLPDDFTGCVIAWNSNNLDSKVGGNYNYLYR |
| | | LFRKSNLKPFERDISTEIYQAGSTPCNGVEGFNCY |
| | | FPLQSYGFQPTNGVGYQPYRVVVLSFELLHAPAT |
| | | VCGPKKSTNLVKNKCVNFNFNGLTGTGVLTESNK |
| | | KFLPFQQFGRDIADTTDAVRDPQTLEILDITPCSFG |
| | | GVSVITPGTNTSNQVAVLYQDVNCTEVPVAIHADQ |
| | | LTPTWRVYSTGSNVFQTRAGCLIGAEHVNNSYEC |
| | | DIPIGAGICASYQTQTNSPRRARSVASQSIIAYTMS |
| | | LGAENSVAYSNNSIAIPTNFTISVTTEILPVSMTKTS |
| | | VDCTMYICGDSTECSNLLLQYGSFCTQLNRALTGI |
| | | AVEQDKNTQEVFAQVKQIYKTPPIKDFGGFNFSQI |
| | | LPDPSKPSKRSFIEDLLFNKVTLADAGFIKQYGDCL |
| | | GDIAARDLICAQKFNGLTVLPPLLTDEMIAQYTSAL |
| | | LAGTITSGWTFGAGAALQIPFAMQMAYRFNGIGVT |
| | | QNVLYENQKLIANQFNSAIGKIQDSLSSTASALGKL |
| | | QDVVNQNAQALNTLVKQLSSNFGAISSVLNDILSR |
| | | LDKVEAEVQIDRLITGRLQSLQTYVTQQLIRAAEIR |
| | | ASANLAATKMSECVLGQSKRVDFCGKGYHLMSF |
| | | PQSAPHGVVFLHVTYVPAQEKNFTTAPAICHDGK |
| | | AHFPREGVFVSNGTHWFVTQRNFYEPQIITTDNTF |
| | | VSGNCDVVIGIVNNTVYDPLQPELDSFKEELDKYF |
| | | KNHTSPDVDLGDISGINASVVNIQKEIDRLNEVAKN |
| | | LNESLIDLQELGKYEQYIKWPWYIWLGFIAGLIAIV |
| | | MVTIMLCCMTSCCSCLKGCCSCGSCCKFDEDDS |
| | | EPVLKGVKLHYT |
| 22 | Beta RBD human | SNLCPFGEVFNATRFASVYAWNRKRISNCVADYS |
| | IgG1 Fc-fusion | VLYNSASFSTFKCYGVSPTKLNDLCFTNVYADSFV |
| | monomer | IRGDEVRQIAPGQTGNIADYNYKLPDDFTGCVIAW |
| | Linker sequence shown in | NSNNLDSKVGGNYNYLYRLFRKSNLKPFERDISTE |
| | bold. | IYQAGSTPCNGVKGFNCYFPLQSYGFQPTYGVGY |
| | | QPYRVVVLSFELLHAPATVCGPGSGSGTHTCPPC |
| | | PAPELLGGPSVFLFPPKPKDTLMISRTPEVTCVVV |
| | | DVSHEDPEVKFNWYVDGVEVHNAKTKPREEQYN |
| | | STYRVVSVLTVLHQDWLNGKEYKCKVSNKALPAP |
| | | IEKTISKAKGQPREPQVYTLPPSREEMTKNQVSLT |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPGK |
| 23 | RBD-human IgG1 | SNLCPFGEVFNATRFASVYAWNRKRISNCVADYS |
| | Fc-fusion monomer | VLYNSASFSTFKCYGVSPTKLNDLCFTNVYADSFV |
| | amino acid | IRGDEVRQIAPGQTGKIADYNYKLPDDFTGCVIAW |
| | sequence | NSNNLDSKVGGNYNYLYRLFRKSNLKPFERDISTE |
| | Linker sequence shown in | IYQAGSTPCNGVEGFNCYFPLQSYGFQPTNGVGY |
| | bold. | QPYRVVVLSFELLHAPATVCGPGSGSGTHTCPPC |
| | | PAPELLGGPSVFLFPPKPKDTLMISRTPEVTCVVV |
| | | DVSHEDPEVKFNWYVDGVEVHNAKTKPREEQYN |
| | | STYRVVSVLTVLHQDWLNGKEYKCKVSNKALPAP |
| | | IEKTISKAKGQPREPQVYTLPPSREEMTKNQVSLT |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPGK |
| 24 | RBD monomer | SNLCPFGEVFNATRFASVYAWNRKRISNCVADYS |
| | amino acid | VLYNSASFSTFKCYGVSPTKLNDLCFTNVYADSFV |
| | sequence | IRGDEVRQIAPGQTGKIADYNYKLPDDFTGCVIAW |
| | 6x His-Tag shown in bold. | NSNNLDSKVGGNYNYLYRLFRKSNLKPFERDISTE |
| | | IYQAGSTPCNGVEGFNCYFPLQSYGFQPTNGVGY |
| | | QPYRVVVLSFELLHAPATVCGPGSHHHHHH |
| 25 | RBD monomer | SNLCPFGEVFNATRFASVYAWNRKRISNCVADYS |
| | amino acid | VLYNSASFSTFKCYGVSPTKLNDLCFTNVYADSFV |
| | sequence without | IRGDEVRQIAPGQTGKIADYNYKLPDDFTGCVIAW |
| | His-tag | NSNNLDSKVGGNYNYLYRLFRKSNLKPFERDISTE |
| | | IYQAGSTPCNGVEGFNCYFPLQSYGFQPTNGVGY |
| | | QPYRVVVLSFELLHAPATVCGP |
| 26 | RBD tandem dimer | SRVQPTESIVRFPNITNLCPFGEVFNATRFASVYA |
| | amino acid | WNRKRISNCVADYSVLYNSASFSTFKCYGVSPTK |
| | sequence | LNDLCFTNVYADSFVIRGDEVRQIAPGQTGKIADY |
| | 6x His-Tag shown in bold. | NYKLPDDFTGCVIAWNSNNLDSKVGGNYNYLYRL |
| | | FRKSNLKPFERDISTEIYQAGSTPCNGVEGENCYF |
| | | PLQSYGFQPTNGVGYQPYRVVVLSFELLHAPATV |
| | | CGPKKSTNLVKNKRVQPTESIVRFPNITNLCPFGE |
| | | VFNATRFASVYAWNRKRISNCVADYSVLYNSASF |
| | | STFKCYGVSPTKLNDLCFTNVYADSFVIRGDEVR |
| | | QIAPGQTGKIADYNYKLPDDFTGCVIAWNSNNLDS |
| | | KVGGNYNYLYRLFRKSNLKPFERDISTEIYQAGST |
| | | PCNGVEGFNCYFPLQSYGFQPTNGVGYQPYRVV |
| | | VLSFELLHAPATVCGPKKSTNLVKNKGSHHHHHH |
| 27 | RBD tandem dimer | SRVQPTESIVRFPNITNLCPFGEVFNATRFASVYA |
| | amino acid | WNRKRISNCVADYSVLYNSASFSTFKCYGVSPTK |
| | sequence without | LNDLCFTNVYADSFVIRGDEVRQIAPGQTGKIADY |
| | His-tag | NYKLPDDFTGCVIAWNSNNLDSKVGGNYNYLYRL |
| | | FRKSNLKPFERDISTEIYQAGSTPCNGVEGFNCYF |
| | | PLQSYGFQPTNGVGYQPYRVVVLSFELLHAPATV |
| | | CGPKKSTNLVKNKRVQPTESIVRFPNITNLCPFGE |
| | | VFNATRFASVYAWNRKRISNCVADYSVLYNSASF |
| | | STFKCYGVSPTKLNDLCFTNVYADSFVIRGDEVR |
| | | QIAPGQTGKIADYNYKLPDDFTGCVIAWNSNNLDS |
| | | KVGGNYNYLYRLFRKSNLKPFERDISTEIYQAGST |
| | | PCNGVEGFNCYFPLQSYGFQPTNGVGYQPYRVV |
| | | VLSFELLHAPATVCGPKKSTNLVKNK |
| 28 | Beta variant RBD | NLCPFGEVFNATRFASVYAWNRKRISNCVADYSV |
| | (N334-P527) | LYNSASFSTFKCYGVSPTKLNDLCFTNVYADSFVI |
| | amino acid | RGDEVRQIAPGQTGNIADYNYKLPDDFTGCVIAW |
| | sequence | NSNNLDSKVGGNYNYLYRLFRKSNLKPFERDISTE |
| | | IYQAGSTPCNGVKGFNCYFPLQSYGFQPTYGVGY |
| | | QPYRVVVLSFELLHAPATVCGP |
| 29 | Beta RBD-human | atgcctgcgctgctctccctcgtgagccttctctccgtgctgctgatgggtt |
| | IgG1 Fc-fusion | gcgtagctgaagctagcaatctgtgccccttcggcgaggtgttcaacg |
| | nucleotide | ccacaagattcgcctctgtgtacgcctggaaccggaagcggatcagc |
| | sequence with | aattgcgtggccgactacagcgtgctgtacaacagcgccagcttcag |
| | leader sequence | caccttcaagtgctacggcgtgtcccctaccaagctgaacgacctgtg |
| | | cttcaccaacgtgtacgccgacagcttcgtgatcagaggcgacgaag |
| | | tgcggcagattgcccctggacagacaggcaacatcgccgattacaa |
| | | ctacaagctgcccgacgacttcaccggctgtgtgattgcctggaacag |
| | | caacaacctggacagcaaagtcggcggcaactacaactacctgtac |
| | | cggctgttccgcaagagcaacctgaagcctttcgagcgggacatcag |
| | | caccgaaatctaccaggccggcagcaccccttgcaatggcgtgaaa |
| | | ggcttcaactgctacttcccactgcaaagctacggcttccagcctacat |
| | | acggcgtgggctaccagccttacagagtggtggtgctgagcttcgag |
| | | ctgctgcatgctcctgccacagtgtgtggacctggatccggcagcggt |
| | | acccacacctgtcctccatgtcctgctccagaactgctcggcggacctt |
| | | ccgtgttcctgtttcctccaaagcctaaggacaccctgatgatcagcag |
| | | aacccctgaagtgacctgcgtggtggtggatgtgtctcacgaggaccc |
| | | cgaagtgaagttcaattggtacgtggacggcgtggaagtgcacaac |
| | | gccaagaccaagcctagagaggaacagtacaacagcacctacag |
| | | agtggtgtccgtgctgaccgtgctgcaccaggattggctgaacggca |
| | | aagagtacaagtgcaaggtgtccaacaaggccctgcctgctcctatc |
| | | gagaaaaccatcagcaaggccaagggccagcctagggaacccca |
| | | ggtttacacactgcctccaagccgggaagagatgaccaagaaccag |
| | | gtgtccctgacctgcctggtcaagggcttttaccccagcgacattgccg |
| | | tggaatgggagagcaatggccagcctgagaacaactacaagacca |
| | | cacctcctgtgctggacagcgacggctcattcttcctgtacagcaagct |
| | | gacagtggacaagagcagatggcagcagggcaacgtgttcagctg |
| | | ctccgtgatgcacgaggccctgcacaaccactacacccagaagtcc |
| | | ctgagcctgtctcctggcaagtga |
| 30 | Beta RBD-human | agcaatctgtgccccttcggcgaggtgttcaacgccacaagattcgcc |
| | IgG1 | tctgtgtacgcctggaaccggaagcggatcagcaattgcgtggccga |
| | Fc-fusion | ctacagcgtgctgtacaacagcgccagcttcagcaccttcaagtgcta |
| | nucleotide | cggcgtgtcccctaccaagctgaacgacctgtgcttcaccaacgtgta |
| | sequence without | cgccgacagcttcgtgatcagaggcgacgaagtgcggcagattgcc |
| | leader sequence | cctggacagacaggcaacatcgccgattacaactacaagctgcccg |
| | | acgacttcaccggctgtgtgattgcctggaacagcaacaacctggac |
| | | agcaaagtcggcggcaactacaactacctgtaccggctgttccgcaa |
| | | gagcaacctgaagcctttcgagcgggacatcagcaccgaaatctac |
| | | caggccggcagcaccccttgcaatggcgtgaaaggcttcaactgcta |
| | | cttcccactgcaaagctacggcttccagcctacatacggcgtgggcta |
| | | ccagccttacagagtggtggtgctgagcttcgagctgctgcatgctcct |
| | | gccacagtgtgtggacctggatccggcagcggtacccacacctgtcc |
| | | tccatgtcctgctccagaactgctcggcggaccttccgtgttcctgtttcct |
| | | ccaaagcctaaggacaccctgatgatcagcagaacccctgaagtga |
| | | cctgcgtggtggtggatgtgtctcacgaggaccccgaagtgaagttca |
| | | attggtacgtggacggcgtggaagtgcacaacgccaagaccaagc |
| | | ctagagaggaacagtacaacagcacctacagagtggtgtccgtgct |
| | | gaccgtgctgcaccaggattggctgaacggcaaagagtacaagtgc |
| | | aaggtgtccaacaaggccctgcctgctcctatcgagaaaaccatcag |
| | | caaggccaagggccagcctagggaaccccaggtttacacactgcct |
| | | ccaagccgggaagagatgaccaagaaccaggtgtccctgacctgc |
| | | ctggtcaagggcttttaccccagcgacattgccgtggaatgggagag |
| | | caatggccagcctgagaacaactacaagaccacacctcctgtgctg |
| | | gacagcgacggctcattcttcctgtacagcaagctgacagtggacaa |
| | | gagcagatggcagcagggcaacgtgttcagctgctccgtgatgcac |
| | | gaggccctgcacaaccactacacccagaagtccctgagcctgtctcc |
| | | tggcaagtga |
| 31 | RBD-human IgG1 | atgcctgcgctgctctccctcgtgagccttctctccgtgctgctgatgggtt |
| | Fc-fusion | gcgtagctgaagctagcaatctgtgccccttcggcgaggtgttcaacg |
| | nucleotide | ccacaagattcgcctctgtgtacgcctggaaccggaagcggatcagc |
| | sequence | aattgcgtggccgactacagcgtgctgtacaacagcgccagcttcag |
| | with | caccttcaagtgctacggcgtgtcccctaccaagctgaacgacctgtg |
| | leader sequence | cttcaccaacgtgtacgccgacagcttcgtgatcagaggcgacgaag |
| | | tgcggcagattgcccctggacagacaggcaagatcgccgattacaa |
| | | ctacaagctgcccgacgacttcaccggctgtgtgattgcctggaacag |
| | | caacaacctggacagcaaagtcggcggcaactacaactacctgtac |
| | | cggctgttccgcaagagcaacctgaagcctttcgagcgggacatcag |
| | | caccgaaatctaccaggccggcagcaccccttgcaatggcgtggaa |
| | | ggcttcaactgctacttcccactgcaaagctacggcttccagcctacaa |
| | | acggcgtgggctaccagccttacagagtggtggtgctgagcttcgag |
| | | ctgctgcatgctcctgccacagtgtgtggacctggatccggcagcggt |
| | | acccacacctgtcctccatgtcctgctccagaactgctcggcggacctt |
| | | ccgtgttcctgtttcctccaaagcctaaggacaccctgatgatcagcag |
| | | aacccctgaagtgacctgcgtggtggtggatgtgtctcacgaggaccc |
| | | cgaagtgaagttcaattggtacgtggacggcgtggaagtgcacaac |
| | | gccaagaccaagcctagagaggaacagtacaacagcacctacag |
| | | agtggtgtccgtgctgaccgtgctgcaccaggattggctgaacggca |
| | | aagagtacaagtgcaaggtgtccaacaaggccctgcctgctcctatc |
| | | gagaaaaccatcagcaaggccaagggccagcctagggaacccca |
| | | ggtttacacactgcctccaagccgggaagagatgaccaagaaccag |
| | | gtgtccctgacctgcctggtcaagggcttttaccccagcgacattgccg |
| | | tggaatgggagagcaatggccagcctgagaacaactacaagacca |
| | | cacctcctgtgctggacagcgacggctcattcttcctgtacagcaagct |
| | | gacagtggacaagagcagatggcagcagggcaacgtgttcagctg |
| | | ctccgtgatgcacgaggccctgcacaaccactacacccagaagtcc |
| | | ctgagcctgtctcctggcaagtga |
| 32 | RBD-human IgG1 | agcaatctgtgccccttcggcgaggtgttcaacgccacaagattcgcc |
| | Fc-fusion | tctgtgtacgcctggaaccggaagcggatcagcaattgcgtggccga |
| | nucleotide | ctacagcgtgctgtacaacagcgccagcttcagcaccttcaagtgcta |
| | sequence without | cggcgtgtcccctaccaagctgaacgacctgtgcttcaccaacgtgta |
| | leader sequence | cgccgacagcttcgtgatcagaggcgacgaagtgcggcagattgcc |
| | | cctggacagacaggcaagatcgccgattacaactacaagctgcccg |
| | | acgacttcaccggctgtgtgattgcctggaacagcaacaacctggac |
| | | agcaaagtcggcggcaactacaactacctgtaccggctgttccgcaa |
| | | gagcaacctgaagcctttcgagcgggacatcagcaccgaaatctac |
| | | caggccggcagcaccccttgcaatggcgtggaaggcttcaactgcta |
| | | cttcccactgcaaagctacggcttccagcctacaaacggcgtgggct |
| | | accagccttacagagtggtggtgctgagcttcgagctgctgcatgctcc |
| | | tgccacagtgtgtggacctggatccggcagcggtacccacacctgtc |
| | | ctccatgtcctgctccagaactgctcggcggaccttccgtgttcctgtttc |
| | | ctccaaagcctaaggacaccctgatgatcagcagaacccctgaagt |
| | | gacctgcgtggtggtggatgtgtctcacgaggaccccgaagtgaagtt |
| | | caattggtacgtggacggcgtggaagtgcacaacgccaagaccaa |
| | | gcctagagaggaacagtacaacagcacctacagagtggtgtccgtg |
| | | ctgaccgtgctgcaccaggattggctgaacggcaaagagtacaagt |
| | | gcaaggtgtccaacaaggccctgcctgctcctatcgagaaaaccatc |
| | | agcaaggccaagggccagcctagggaaccccaggtttacacactg |
| | | cctccaagccgggaagagatgaccaagaaccaggtgtccctgacct |
| | | gcctggtcaagggcttttaccccagcgacattgccgtggaatgggag |
| | | agcaatggccagcctgagaacaactacaagaccacacctcctgtgc |
| | | tggacagcgacggctcattcttcctgtacagcaagctgacagtggaca |
| | | agagcagatggcagcagggcaacgtgttcagctgctccgtgatgca |
| | | cgaggccctgcacaaccactacacccagaagtccctgagcctgtctc |
| | | ctggcaagtga |
| 33 | Beta RBD human | MGILPSPGMPALLSLVSLLSVLLMGCVAEA [symbol] SNLC |
| | IgG1 Fc-fusion | PFGEVFNATRFASVYAWNRKRISNCVADYSVLYN |
| | monomer | SASFSTFKCYGVSPTKLNDLCFTNVYADSFVIRGD |
| | pHLSec leader sequence | EVRQIAPGQTGNIADYNYKLPDDFTGCVIAWNSN |
| | shown in underline | NLDSKVGGNYNYLYRLFRKSNLKPFERDISTEIYQ |
| | Linker sequence shown in | AGSTPCNGVKGFNCYFPLQSYGFQPTYGVGYQP |
| | bold. | YRVVVLSFELLHAPATVCGPGSGSGTHTCPPCPA |
| | \hat{ } refers to the predicted | PELLGGPSVFLFPPKPKDTLMISRTPEVTCVVVDV |
| | cleavage site based on | SHEDPEVKFNWYVDGVEVHNAKTKPREEQYNST |
| | SignalP5.0. | YRVVSVLTVLHQDWLNGKEYKCKVSNKALPAPIE |
| | | KTISKAKGQPREPQVYTLPPSREEMTKNQVSLTC |
| | | LVKGFYPSDIAVEWESNGQPENNYKTTPPVLDSD |
| | | GSFFLYSKLTVDKSRWQQGNVFSCSVMHEALHN |
| | | HYTQKSLSLSPGK |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The general chemical terms used in the formulae herein have their usual meaning.

The inventors have developed a novel recombinant RBD chimeric or fusion protein vaccine which when combined with adjuvant (e.g. α-GalCer or PEG-R4-Pam-2-Cys) elicits a strong neutralising antibody response which is broadly protective to a range of SARS-COV-2 strains. Notably, the inventors have shown that vaccination via various routes, including the upper respiratory tract via intranasal administration, of the vaccine composition with adjuvant (e.g. α-GalCer or PEG-R4-Pam-2-Cys) elicits a strong neutralising antibody response which is protective from viral replication in both the upper (nasal cavity) and lower respiratory tract. The broad protection provides protection from disseminated lung infection which can result in potentially deadly complications. Furthermore, the inventors have found that the immunity induced by vaccination lasts at least 3 months, and that broad protection is maintained to escape mutant strains.

### Coronavirus

The term “coronavirus” or “CoV” refers to any virus of the coronavirus family, including but not limited to SARS-COV-2, MERS-COV, and SARS-COV as well as endemic coronaviruses such as HCoV-NL63, HCOV-229E, HCoV-OC43 and HKU1. SARS-COV-2 refers to the newly emerged coronavirus which was identified as the cause of the serious outbreak starting in Wuhan, China, and which has rapidly spread to other areas of the globe. SARS-COV-2 has also been known as 2019-nCOV and Wuhan coronavirus. It binds via the viral spike protein to human host cell receptor angiotensin-converting enzyme 2 (ACE2). The spike protein also binds to and is cleaved by TMPRSS2, which activates the spike protein for membrane fusion of the virus.

The subfamily Coronavirinae in the family Coronaviridae and the order Nidovirales (International Committee on Taxonomy of Viruses). This subfamily consists of four genera, Alphacoronavirus, Betacoronavirus, Gammacoronavirus and Deltacoronavirus, on the basis of their phylogenetic relationships and genomic structures. Subgroup clusters are labeled as 1a and 1b for the Alphacoronavirus and 2a, 2b, 2c, and 2d for the Betacoronavirus. The alphacoronaviruses and betacoronaviruses infect only mammals. The gammacoronaviruses and deltacoronaviruses infect birds, but some of them can also infect mammals. Alphacoronaviruses and betacoronaviruses usually cause respiratory illness in humans and gastroenteritis in animals. The three highly pathogenic viruses, SARS-COV, MERS-CoV and SARS-COV-2, cause severe respiratory syndrome in humans, and the other four human coronaviruses (HCoV-NL63, HCoV-229E, HCoV-OC43 and HKU1) induce only mild upper respiratory diseases in immunocompetent hosts, although some of them can cause severe infections in infants, young children and elderly individuals. Alphacoronaviruses and betacoronaviruses can pose a heavy disease burden on livestock; these viruses include porcine transmissible gastroenteritis virus, porcine enteric diarrhoea virus (PEDV) and the recently emerged swine acute diarrhoea syndrome coronavirus (SADS-COV). On the basis of current sequence databases, all human coronaviruses have animal origins: SARS-COV, MERS-COV, SARS-COV-2, HCoV-NL63 and HCoV-229E are considered to have originated in bats; HCoV-OC43 and HKU1 likely originated from rodents.

The coronaviruses include antigenic groups I, II, and III. Nonlimiting examples of coronaviruses include SARS coronavirus, MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as any others described herein, and including those referred to in Cui, et al. Nature Reviews Microbiology volume 17, pages 181-192 (2019), and Shereen et al. Journal of Advanced Research, Volume 24, July 2020 (published online 16 Mar. 2020), Pages 91-98.

Non-limiting examples of a subgroup 1a coronavirus include FCov.FIPV.79.1146.VR.2202 (GenBank Accession No. NV_007025), transmissible gastroenteritis virus (TGEV) (GenBank Accession No. NC_002306; GenBank Accession No. Q811789.2; GenBank Accession No. DQ811786.2; GenBank Accession No. DQ811788.1; GenBank Accession No. DQ811785.1; GenBank Accession No. X52157.1; GenBank Accession No. AJ011482.1; GenBank Accession No. KC962433.1; GenBank Accession No. AJ271965.2; GenBank Accession No. JQ693060.1; GenBank Accession No. KC609371.1; GenBank Accession No. JQ693060.1; GenBank Accession No. JQ693059.1; GenBank Accession No. JQ693058.1; GenBank Accession No. JQ693057.1; GenBank Accession No. JQ693052.1; GenBank Accession No. JQ693051.1; GenBank Accession No. JQ693050.1), porcine reproductive and respiratory syndrome virus (PRRSV) (GenBank Accession No. NC_001961.1; GenBank Accession No. DQ811787), as well as any other subgroup 1a coronavirus now known (e.g., as can be found in the GenBank® Database) or later identified, and any combination thereof.

Non-limiting examples of a subgroup 1b coronavirus include BtCoV.1A.AFCD62 (GenBank Accession No. NC_010437), BtCoV.1B.AFCD307 (GenBank Accession No. NC_010436), BtCov.HKU8.AFCD77 (GenBank Accession No. NC_010438), BtCoV.512.2005 (GenBank Accession No. DQ648858), porcine epidemic diarrhea virus PEDV.CV777 (GenBank Accession No. NC_003436, GenBank Accession No. DQ355224.1, GenBank Accession No. DQ355223.1, GenBank Accession No. DQ355221.1, GenBank Accession No. JN601062.1, GenBank Accession No. N601061.1, GenBank Accession No. JN601060.1, GenBank Accession No. JN601059.1, GenBank Accession No. JN601058.1, GenBank Accession No. JN601057.1, GenBank Accession No. JN601056.1, GenBank Accession No. JN601055.1, GenBank Accession No. JN601054.1, GenBank Accession No. JN601053.1, GenBank Accession No. JN601052.1, GenBank Accession No. JN400902.1, GenBank Accession No. JN547395.1, GenBank Accession No. FJ687473.1, GenBank Accession No. FJ687472.1, GenBank Accession No. FJ687471.1, GenBank Accession No. FJ687470.1, GenBank Accession No. FJ687469.1, GenBank Accession No. FJ687468.1, GenBank Accession No. FJ687467.1, GenBank Accession No. FJ687466.1, GenBank Accession No. FJ687465.1, GenBank Accession No. FJ687464.1, GenBank Accession No. FJ687463.1, GenBank Accession No. FJ687462.1, GenBank Accession No. FJ687461.1, GenBank Accession No. FJ687460.1, GenBank Accession No. FJ687459.1, GenBank Accession No. FJ687458.1, GenBank Accession No. FJ687457.1, GenBank Accession No. FJ687456.1, GenBank Accession No. FJ687455.1, GenBank Accession No. FJ687454.1, GenBank Accession No. FJ687453 GenBank Accession No. FJ687452.1, GenBank Accession No. FJ687451.1, GenBank Accession No. FJ687450.1, GenBank Accession No. FJ687449.1, GenBank Accession No. AF500215.1, GenBank Accession No. KF476061.1, GenBank Accession No. KF476060.1, GenBank Accession No. KF476059.1, GenBank Accession No. KF476058.1, GenBank Accession No. KF476057.1, GenBank Accession No. KF476056.1, GenBank Accession No. KF476055.1, GenBank Accession No. KF476054.1, GenBank Accession No. KF476053.1, GenBank Accession No. KF476052.1, GenBank Accession No. KF476051.1, GenBank Accession No. KF476050.1, GenBank Accession No. KF476049.1, GenBank Accession No. KF476048.1, GenBank Accession No. KF177258.1, GenBank Accession No. KF177257.1, GenBank Accession No. KF177256.1, GenBank Accession No. KF177255.1), HCoV.229E (GenBank Accession No. NC_002645), HCoV.NL63.Amsterdam.I (GenBank Accession No. NC_005831), BtCoV.HKU2.HK.298.2006 (GenBank Accession No. EF203066), BtCoV.HKU2.HK.33.2006 (GenBank Accession No. EF203067), BtCoV.HKU2.HK.46.2006 (GenBank Accession No. EF203065), BtCoV.HKU2.GD.430.2006 (GenBank Accession No. EF203064), as well as any other subgroup 1b coronavirus now known (e.g., as can be found in the GenBank® Database) or later identified, and any combination thereof.

Non-limiting examples of a subgroup 2a coronavirus include HCoV.HKU1.C.N5 (GenBank Accession No. DQ339101), MHV.A59 (GenBank Accession No. NC 001846), PHEV.VW572 (GenBank Accession No. NC 007732), HCoV.OC43.ATCC.VR.759 (GenBank Accession No. NC_005147), bovine enteric coronavirus (BCoV.ENT) (GenBank Accession No. NC_003045), as well as any other subgroup 2a coronavirus now known (e.g., as can be found in the GenBank® Database) or later identified, and any combination thereof.

Non-limiting examples of subgroup 2b coronaviruses include Bat SARS COV (GenBank Accession No. FJ211859), SARS COV (GenBank Accession No. FJ211860), SARS-COV-2 (GenBank Accession No. NC_045512.2), BtSARS.HKU3.1 (GenBank Accession No. DQ022305), BISARS.HKU3.2 (GenBank Accession No. DQ084199), BtSARS.HKU3.3 (GenBank Accession No. DQ084200), BtSARS.Rm1 (GenBank Accession No. DQ412043), BtCoV.279.2005 (GenBank Accession No. DQ648857), BtSARS.Rf1 (GenBank Accession No. DQ412042), BtCoV.273.2005 (GenBank Accession No. DQ648856), BtSARS.Rp3 (GenBank Accession No. DQ071615), SARS CoV.A022 (GenBank Accession No. AY686863), SARSCOV.CUHK-W1 (GenBank Accession No. AY278554), SARSCOV.GDO1 (GenBank Accession No. AY278489), SARSCOV.HC.SZ.61.03 (GenBank Accession No. AY515512), SARSCOV.SZ16 (GenBank Accession No. AY304488), SARSCOV.Urbani (GenBank Accession No. AY278741), SARSCOV.civet010 (GenBank Accession No. AY572035), and SARSCOV.MA.15 (GenBank Accession No. DQ497008), Rs SHC014 (GenBank® Accession No. KC881005), Rs3367 (GenBank® Accession No. KC881006), WiV1 S (GenBank® Accession No. KC881007) as well as any other subgroup 2b coronavirus now known (e.g., as can be found in the GenBank® Database) or later identified, and any combination thereof.

Non-limiting examples of subgroup 2c coronaviruses include: Middle East Respiratory Syndrome coronavirus isolate Riyadh_2_2012 (GenBank Accession No. KF600652.1), Middle East respiratory syndrome coronavirus isolate Al-Hasa_18_2013 (GenBank Accession No. KF600651.1), Middle East respiratory syndrome coronavirus isolate Al-Hasa_17_2013 (GenBank Accession No. KF600647.1), Middle East respiratory syndrome coronavirus isolate Al-Hasa_15_2013 (GenBank Accession No. KF600645.1), Middle East respiratory syndrome coronavirus isolate Al-Hasa_16_2013 (GenBank Accession No. KF600644.1), Middle East respiratory syndrome coronavirus isolate Al-Hasa_21_2013 (GenBank Accession No. KF600634), Middle East respiratory syndrome coronavirus isolate Al-Hasa_19_2013 (GenBank Accession No. KF600632), Middle East respiratory syndrome coronavirus isolate Buraidah_1_2013 (GenBank Accession No. KF600630.1), Middle East respiratory syndrome coronavirus isolate Hafr-Al-Batin_1_2013 (GenBank Accession No. KF600628.1), Middle East respiratory syndrome coronavirus isolate Al-Hasa_12_2013 (GenBank Accession No. KF600627.1), Middle East respiratory syndrome coronavirus isolate Bisha_1_2012 (GenBank Accession No. KF600620.1), Middle East respiratory syndrome coronavirus isolate Riyadh_3_2013 (GenBank Accession No. KF600613.1), Middle East respiratory syndrome coronavirus isolate Riyadh_1_2012 (GenBank Accession No. KF600612.1), Middle East respiratory syndrome coronavirus isolate Al-Hasa_3_2013 (GenBank Accession No. KF186565.1), Middle East respiratory syndrome coronavirus isolate Al-Hasa_1_2013 (GenBank Accession No. KF186567.1), Middle East respiratory syndrome coronavirus isolate Al-Hasa_2_2013 (GenBank Accession No. KF186566.1), Middle East respiratory syndrome coronavirus isolate Al-Hasa_4_2013 (GenBank Accession No. KF186564.1), Middle East respiratory syndrome coronavirus (GenBank Accession No. KF192507.1), Betacoronavirus England 1-N1 (GenBank Accession No. NC_019843), MERS-COV_SA-N1 (GenBank Accession No. KC667074), following isolates of Middle East Respiratory Syndrome Coronavirus (GenBank Accession No: KF600656.1, GenBank Accession No: KF600655.1, GenBank Accession No: KF600654.1, GenBank Accession No: KF600649.1, GenBank Accession No: KF600648.1, GenBank Accession No: KF600646.1, GenBank Accession No: KF600643.1, GenBank Accession No: KF600642.1, GenBank Accession No: KF600640.1, GenBank Accession No: KF600639.1, GenBank Accession No: KF600638.1, GenBank Accession No: KF600637.1, GenBank Accession No: KF600636.1, GenBank Accession No: KF600635.1, GenBank Accession No: KF600631.1, GenBank Accession No: KF600626.1, GenBank Accession No: KF600625.1, GenBank Accession No: KF600624.1, GenBank Accession No: KF600623.1, GenBank Accession No: KF600622.1, GenBank Accession No: KF600621.1, GenBank Accession No: KF600619.1, GenBank Accession No: KF600618.1, GenBank Accession No: KF600616.1, GenBank Accession No: KF600615.1, GenBank Accession No: KF600614.1, GenBank Accession No: KF600641.1, GenBank Accession No: KF600633.1, GenBank Accession No: KF600629.1, GenBank Accession No: KF600617.1), Coronavirus Neoromicia/PML-PHE1/RSA/2011 GenBank Accession: KC869678.2, Bat Coronavirus Taper/CII_KSA_287/Bisha/Saudi Arabia/GenBank Accession No: KF493885.1, Bat coronavirus Rhhar/CII_KSA_003/Bisha/Saudi Arabia/2013 GenBank Accession No: KF493888.1, Bat coronavirus Pikuh/CII_KSA_001/Riyadh/Saudi Arabia/2013 GenBank Accession No: KF493887.1, Bat coronavirus Rhhar/CII_KSA_002/Bisha/Saudi Arabia/2013 GenBank Accession No: KF493886.1, Bat Coronavirus Rhhar/CII_KSA_004/Bisha/Saudi Arabia/2013 GenBank Accession No: KF493884.1, BtCoV.HKU4.2 (GenBank Accession No. EF065506), BtCoV.HKU4.1 (GenBank Accession No. NC_009019), BtCoV.HKU4.3 (GenBank Accession No. EF065507), BtCoV.HKU4.4 (GenBank Accession No. EF065508), BtCoV 133.2005 (GenBank Accession No. NC 008315), BtCoV.HKU5.5 (GenBank Accession No. EF065512); BtCoV.HKU5.1 (GenBank Accession No. NC_009020), BtCoV.HKU5.2 (GenBank Accession No. EF065510), BtCoV.HKU5.3 (GenBank Accession No. EF065511), human betacoronavirus 2c Jordan-N3/2012 (GenBank Accession No. KC776174.1; human betacoronavirus 2c EMC/2012 (GenBank Accession No. JX869059.2), Pipistrellus bat coronavirus HKU5 isolates (GenBank Accession No: KC522089.1, GenBank Accession No: KC522088.1, GenBank Accession No: KC522087.1, GenBank Accession No: KC522086.1, GenBank Accession No: KC522085.1, GenBank Accession No: KC522084.1, GenBank Accession No: KC522083.1, GenBank Accession No: KC522082.1, GenBank Accession No: KC522081.1, GenBank Accession No: KC522080.1, GenBank Accession No: KC522079.1, GenBank Accession No: KC522078.1, GenBank Accession No: KC522077.1, GenBank Accession No: KC522076.1, GenBank Accession No: KC522075.1, GenBank Accession No: KC522104.1, GenBank Accession No: KC522104.1, GenBank Accession No: KC522103.1, GenBank Accession No: KC522102.1, GenBank Accession No: KC522101.1, GenBank Accession No: KC522100.1, GenBank Accession No: KC522099.1, GenBank Accession No: KC522098.1, GenBank Accession No: KC522097.1, GenBank Accession No: KC522096.1, GenBank Accession No: KC522095.1, GenBank Accession No: KC522094.1, GenBank Accession No: KC522093.1, GenBank Accession No: KC522092.1, GenBank Accession No: KC522091.1, GenBank Accession No: KC522090.1, GenBank Accession No: KC522119.1 GenBank Accession No: KC522118.1 GenBank Accession No: KC522117.1 GenBank Accession No: KC522116.1 GenBank Accession No: KC522115.1 GenBank Accession No: KC522114.1 GenBank Accession No: KC522113.1 GenBank Accession No: KC522112.1 GenBank Accession No: KC522111.1 GenBank Accession No: KC522110.1 GenBank Accession No: KC522109.1 GenBank Accession No: KC522108.1, GenBank Accession No: KC522107.1, GenBank Accession No: KC522106.1, GenBank Accession No: KC522105.1) Pipistrellus bat coronavirus HKU4 isolates (GenBank Accession No: KC522048.1, GenBank Accession No: KC522047.1, GenBank Accession No: KC522046.1, GenBank Accession No: KC522045.1, GenBank Accession No: KC522044.1, GenBank Accession No: KC522043.1, GenBank Accession No: KC522042.1, GenBank Accession No: KC522041.1, GenBank Accession No: KC522040.1 GenBank Accession No: KC522039.1, GenBank Accession No: KC522038.1, GenBank Accession No: KC522037.1, GenBank Accession No: KC522036.1, GenBank Accession No: KC522048.1 GenBank Accession No: KC522047.1 GenBank Accession No: KC522046.1 GenBank Accession No: KC522045.1 GenBank Accession No: KC522044.1 GenBank Accession No: KC522043.1 GenBank Accession No: KC522042.1 GenBank Accession No: KC522041.1 GenBank Accession No: KC522040.1, GenBank Accession No: KC522039.1 GenBank Accession No: KC522038.1 GenBank Accession No: KC522037.1 GenBank Accession No: KC522036.1, GenBank Accession No: KC522061.1 GenBank Accession No: KC522060.1 GenBank Accession No: KC522059.1 GenBank Accession No: KC522058.1 GenBank Accession No: KC522057.1 GenBank Accession No: KC522056.1 GenBank Accession No: KC522055.1 GenBank Accession No: KC522054.1 GenBank Accession No: KC522053.1 GenBank Accession No: KC522052.1 GenBank Accession No: KC522051.1 GenBank Accession No: KC522050.1 GenBank Accession No: KC522049.1 GenBank Accession No: KC522074.1, GenBank Accession No: KC522073.1 GenBank Accession No: KC522072.1 GenBank Accession No: KC522071.1 GenBank Accession No: KC522070.1 GenBank Accession No: KC522069.1 GenBank Accession No: KC522068.1 GenBank Accession No: KC522067.1, GenBank Accession No: KC522066.1 GenBank Accession No: KC522065.1 GenBank Accession No: KC522064.1, GenBank Accession No: KC522063.1, or GenBank Accession No: KC522062.1, as well as any other subgroup 2b coronavirus now known (e.g., as can be found in the GenBank® Database) or later identified, and any combination thereof.

Non-limiting examples of a subgroup 2d coronavirus include BtCoV.HKU9.2 (GenBank Accession No. EF065514), BtCoV.HKU9.1 (GenBank Accession No. NC_009021), BtCoV.HKU9.3 (GenBank Accession No. EF065515), BtCoV.HKU9.4 (GenBank Accession No. EF065516), as well as any other subgroup 2d coronavirus now known (e.g., as can be found in the GenBank® Database) or later identified, and any combination thereof.

Non-limiting examples of a subgroup 3 coronavirus include IBV.Beaudette.IBV.p65 (GenBank Accession No. DQ001339), as well as any other subgroup 3 coronavirus now known (e.g., as can be found in the GenBank® Database) or later identified, and any combination thereof.

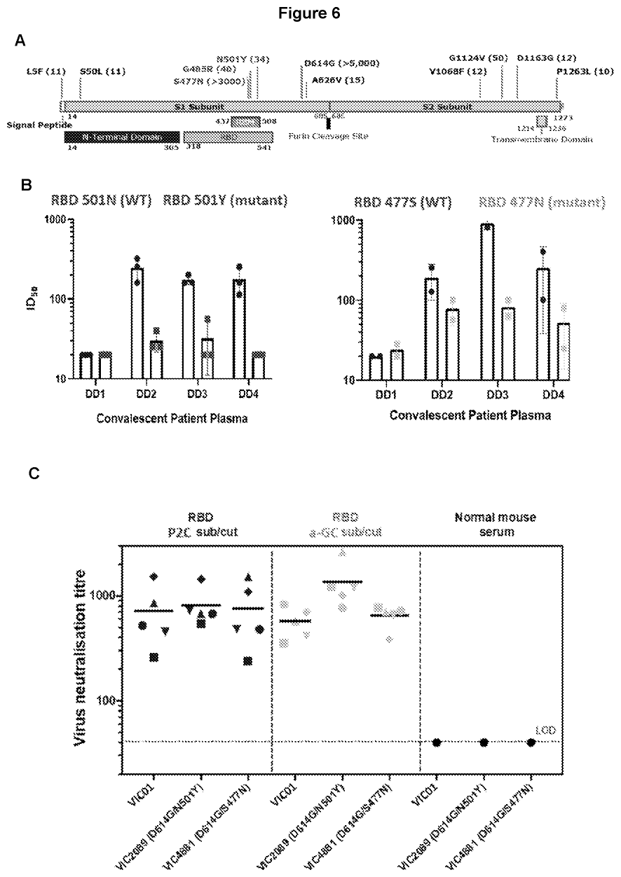

The coronavirus may be any virus that comprises a receptor binding domain of a Spike protein that includes one or more of the mutations as shown in FIG. 6.

A subject or individual in need of treatment according to any aspect of the invention, or requiring administration of any composition described herein, may be an individual who is displaying a symptom of a coronavirus infection or who has been diagnosed with a coronavirus infection. Further, the subject or individual may be one who has been clinically or biochemically determined to be infected with a coronavirus.

A subject may be in a stage of coronavirus infection before end stage-organ failure has developed. A subject in need thereof may be anyone with a coronavirus infection from the onset of clinical progression, before end-organ failure has developed. In one embodiment, the subject has had coronavirus infection symptoms for less than or equal to 12 days, and who does not have life-threatening organ dysfunction or organ failure. Preferably the subject is early in the course of the disease, for example, before day 14 from symptom onset, or during the viremic and seronegative stage.

A “subject” or “individual” can also be any animal that is susceptible to infection by coronavirus and/or susceptible to diseases or disorders caused by coronavirus infection. A subject of this invention can be a mammal and in particular embodiments is a human, which can be an infant, a child, an adult or an elderly adult. A “subject at risk of infection by a coronavirus” or a “subject at risk of coronavirus infection” is any subject who may be or has been exposed to a coronavirus. “Subject” or “individual” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs, or any animal that can be infected by coronavirus.

The subjects at risk include, but are not limited to, an immunocompromised person, an elderly adult (more than 65 years of age), children younger than 2 years of age, healthcare workers, adults or children in close contact with a person(s) with confirmed or suspected coronavirus infection, and people with underlying medical conditions such as pulmonary infection, heart disease or diabetes, primary or secondary immunodeficiency.

### Proteins and Polypeptides

“Isolated,” when used to describe the various polypeptides disclosed herein, means the polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated protein includes polypeptide in situ within recombinant cells, since at least one component of the polypeptide natural environment will not be present. Ordinarily, however, isolated polypeptide will be prepared by at least one purification step.

A “fragment” is a portion of a polypeptide of the present invention that retains substantially similar functional activity or substantially the same biological function or activity as the polypeptide, which can be determined using assays described herein.

“Percent (%) amino acid sequence identity” or “percent (%) identical” with respect to a polypeptide sequence, i.e. a polypeptide of the invention defined herein, is defined as the percentage of amino acid residues in a candidate sequence that are identical with the amino acid residues in the specific polypeptide of the invention, after aligning the sequences and introducing gaps, if necessary, to achieve the maximum percent sequence identity, and not considering any conservative substitutions as part of the sequence identity.

Those skilled in the art can determine appropriate parameters for measuring alignment, including any algorithms (non-limiting examples described below) needed to achieve maximal alignment over the full-length of the sequences being compared. When amino acid sequences are aligned, the percent amino acid sequence identity of a given amino acid sequence A to, with, or against a given amino acid sequence B (which can alternatively be phrased as a given amino acid sequence A that has or comprises a certain percent amino acid sequence identity to, with, or against a given amino acid sequence B) can be calculated as: percent amino acid sequence identity=X/Y100, where X is the number of amino acid residues scored as identical matches by the sequence alignment program's or algorithm's alignment of A and B and Y is the total number of amino acid residues in B. If the length of amino acid sequence A is not equal to the length of amino acid sequence B, the percent amino acid sequence identity of A to B will not equal the percent amino acid sequence identity of B to A.

In calculating percent identity, typically exact matches are counted. The determination of percent identity between two sequences can be accomplished using a mathematical algorithm. A non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, CA). After alignment of amino acid sequences with ClustalW, the percent amino acid identity can be assessed. A non-limiting examples of a software program useful for analysis of ClustalW alignments is GENEDOC™ or JalView (http://www.jalview.org/). GENEDOC™ allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, CA, USA). When utilizing the ALIGN program for comparing amino acid sequences, a PAM 120 weight residue table, a gap length penalty of 12, and a gap penalty of 4 can be used.

The polypeptide desirably comprises an amino end and a carboxyl end. The polypeptide can comprise D-amino acids, L-amino acids or a mixture of D- and L-amino acids. The D-form of the amino acids, however, is particularly preferred since a polypeptide comprised of D-amino acids is expected to have a greater retention of its biological activity in vivo.

The polypeptide can be prepared by any of a number of conventional techniques. The polypeptide can be isolated or purified from a naturally occurring source or from a recombinant source. Recombinant production is preferred. For instance, in the case of recombinant polypeptides, a DNA fragment encoding a desired peptide can be subcloned into an appropriate vector using well-known molecular genetic techniques (see, e.g., Maniatis et al., Molecular Cloning: A Laboratory Manual, 2nd ed. (Cold Spring Harbor Laboratory, 1982); Sambrook et al., Molecular Cloning A Laboratory Manual, 2nd ed. (Cold Spring Harbor Laboratory, 1989). The fragment can be transcribed and the polypeptide subsequently translated in vitro. Commercially available kits also can be employed (e.g., such as manufactured by Clontech, Palo Alto, Calif.; Amersham Pharmacia Biotech Inc., Piscataway, N.J.; InVitrogen, Carlsbad, Calif., and the like). The polymerase chain reaction optionally can be employed in the manipulation of nucleic acids.

The term “conservative substitution” as used herein, refers to the replacement of an amino acid present in the native sequence in the peptide with a naturally or non-naturally occurring amino acid or a peptidomimetic having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

Conservative amino acid substitution tables providing functionally similar amino acids are well known to one of ordinary skill in the art. The following six groups are examples of amino acids that may be considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

As naturally occurring amino acids are typically grouped according to their properties, conservative substitutions by naturally occurring amino acids can be determined bearing in mind the fact that replacement of charged amino acids by sterically similar non-charged amino acids are considered as conservative substitutions. For producing conservative substitutions by non-naturally occurring amino acids it is also possible to use amino acid analogs (synthetic amino acids) well known in the art. A peptidomimetic of the naturally occurring amino acid is well documented in the literature known to the skilled person and non-natural or unnatural amino acids are described further below. When affecting conservative substitutions, the substituting amino acid should have the same or a similar functional group in the side chain as the original amino acid.

The phrase “non-conservative substitution” or a “non-conservative residue” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cyclohexylmethyl glycine for alanine, isoleucine for glycine, or —NH—CH[(—CH2)5-COOH]—CO— for aspartic acid. Non-conservative substitution includes any mutation that is not considered conservative.

A non-conservative amino acid substitution can result from changes in: (a) the structure of the amino acid backbone in the area of the substitution; (b) the charge or hydrophobicity of the amino acid; or (c) the bulk of an amino acid side chain. Substitutions generally expected to produce the greatest changes in protein properties are those in which: (a) a hydrophilic residue is substituted for (or by) a hydrophobic residue; (b) a proline is substituted for (or by) any other residue; (c) a residue having a bulky side chain, e.g., phenylalanine, is substituted for (or by) one not having a bulky side chain, e.g., glycine; or (d) a residue having an electropositive side chain, e.g., lysyl, arginyl, or histidyl group, is substituted for (or by) an electronegative residue, e.g., glutamyl or aspartyl group.

Alterations of the native amino acid sequence to produce mutant polypeptides, such as by insertion, deletion and/or substitution, can be done by a variety of means known to those skilled in the art. For instance, site-specific mutations can be introduced by ligating into an expression vector a synthesized oligonucleotide comprising the modified site. Alternately, oligonucleotide-directed site-specific mutagenesis procedures can be used, such as disclosed in Walder et al., Gene 42:133 (1986); Bauer et al., Gene 37:73 (1985); Craik, Biotechniques, 12-19 (January 1995); and U.S. Pat. Nos. 4,518,584 and 4,737,462. A preferred means for introducing mutations is the QuikChange Site-Directed Mutagenesis Kit (Stratagene, LaJolla, Calif.).

Any appropriate expression vector (e.g., as described in Pouwels et al., Cloning Vectors: A Laboratory Manual (Elsevier, N.Y.: 1985)) and corresponding suitable host can be employed for production of recombinant polypeptides. Expression hosts include, but are not limited to, bacterial species within the genera Escherichia, Bacillus, Pseudomonas, Salmonella, mammalian or insect host cell systems including baculovirus systems (e.g., as described by Luckow et al., Bio/Technology 6:47 (1988)), and established cell lines such as the COS-7, C127, 3T3, CHO, HeLa, and BHK cell lines, and the like. The skilled person is aware that the choice of expression host has ramifications for the type of polypeptide produced. For instance, the glycosylation of polypeptides produced in yeast or mammalian cells (e.g., COS-7 cells) will differ from that of polypeptides produced in bacterial cells, such as Escherichia coli.

Alternately, a polypeptide of the invention can be synthesized using standard peptide synthesizing techniques well-known to those of ordinary skill in the art (e.g., as summarized in Bodanszky, Principles of Peptide Synthesis (Springer-Verlag, Heidelberg: 1984)). In particular, the polypeptide can be synthesized using the procedure of solid-phase synthesis (see, e.g., Merrifield, J. Am. Chem. Soc. 85:2149-54 (1963); Barany et al., Int. J. Peptide Protein Res. 30:705-739 (1987); and U.S. Pat. No. 5,424,398). If desired, this can be done using an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The polypeptide-containing mixture can then be extracted, for instance, with dimethyl ether, to remove non-peptidic organic compounds, and the synthesized polypeptide can be extracted from the resin powder (e.g., with about 25% w/v acetic acid). Following the synthesis of the polypeptide, further purification (e.g., using high performance liquid chromatography (HPLC)) optionally can be done in order to eliminate any incomplete polypeptides or free amino acids. Amino acid and/or HPLC analysis can be performed on the synthesized polypeptide to validate its identity. For other applications according to the invention, it may be preferable to produce the polypeptide as part of a larger fusion protein, such as by the methods described herein or other genetic means, or as part of a larger conjugate, such as through physical or chemical conjugation, as known to those of ordinary skill in the art and described herein.

A polypeptide of the invention may also be modified by, conjugated or fused to another moiety to facilitate purification, or increasing the in vivo half-life of the polypeptides, or for use in immunoassays using methods known in the art. For example, a polypeptide of the invention may be modified by glycosylation, acetylation, pegylation, phosphorylation, amidation, derivatization by known protecting/blocking groups, proteolytic cleavage, linkage to a cellular ligand or other protein, etc.

A “peptidomimetic” is a synthetic chemical compound that has substantially the same structure and/or functional characteristics of a polypeptide of the invention, the latter being described further herein. Typically, a peptidomimetic has the same or similar structure as a polypeptide of the invention, for example the same or similar sequence of SEQ ID NO: 6 or 23 or fragment thereof that has a reduced capacity to form a dimer. A peptidomimetic generally contains at least one residue that is not naturally synthesised. Non-natural components of peptidomimetic compounds may be according to one or more of: a) residue linkage groups other than the natural amide bond (‘peptide bond’) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

Peptidomimetics can be synthesized using a variety of procedures and methodologies described in the scientific and patent literatures, e.g., Organic Syntheses Collective Volumes, Gilman et al. (Eds) John Wiley & Sons, Inc., NY, al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymot. 267:220-234

Modifications contemplated herein include, but are not limited to, modification to side chains, incorporating of unnatural amino acids and/or their derivatives during polypeptide synthesis and the use of crosslinkers and other methods which impose conformational constraints on the polypeptides of the invention. Any modification, including post-translational modification that reduces the capacity of the molecule to form a dimer is contemplated herein. An example includes modification incorporated by click chemistry as known in the art. Exemplary modifications include PEGylation and glycosylation.

Examples of side chain modifications contemplated by the present invention include modifications of amino groups such as by reductive alkylation by reaction with an aldehyde followed by reduction with NaBH4; amidation with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH4.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 2.

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl) glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyl-α-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH2) n spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The term “antibody” refers to various antibody structures, including but not limited to monoclonal antibodies, polyclonal antibodies, multispecific antibodies. The term “antibody” may also be used interchangeably with the term “immunoglobulin”.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Particularly, the term “recombinant human antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Thus, such recombinant human antibodies have variable and constant regions derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

### Spike Protein and RBD

Coronavirus virions are spherical with diameters of approximately 80-200 nm. The most prominent feature of coronaviruses is the club-shape spike projections emanating from the surface of the virion. These spikes are a defining feature of the virion and give them the appearance of a solar corona, prompting the name, coronaviruses. Within the envelope of the virion is the nucleocapsid. Coronaviruses have helically symmetrical nucleocapsids, which is uncommon among positive-sense RNA viruses, but far more common for negative-sense RNA viruses. SARS-COV-2, MERS-COV, and SARS-COV belong to the coronavirus family.

The term “CoV-S” also called “S” or “S protein” or “spike protein” of the coronavirus and can refer to specific S proteins such as SARS-COV-2-S, MERS-COV S, and SARS-COV S or other members of the coronavirus family. The SARS-COV-2 spike protein is a 1273 amino acid type I membrane glycoprotein which assembles into trimers that constitute the spikes or peplomers on the surface of the enveloped coronavirus particle. The protein has two essential functions, (1) host receptor binding and (2) membrane fusion, which are attributed to the N-terminal (S1) and C-terminal (S2) halves of the S protein. CoV-S binds to its cognate receptor via a receptor binding domain (RBD) present in the S1 subunit. The amino acid sequence of the full-length SARS-COV-2 spike protein is exemplified by the amino acid sequence provided in SEQ ID NO: 21. The term “CoV-S” includes protein variants of CoV spike protein isolated from different CoV spike protein or a fragment thereof. The term also encompasses CoV spike protein or a fragment thereof coupled to, for example, a histidine tag, mouse or human Fc, or a signal sequence such as ROR1.

In any embodiment, the Spike protein may be from a variant or strain as described herein, including in Table 3 below.

In any aspect, a receptor binding domain may be from a SARS-COV-2 variant, such as those described in Table 3 below. SARS-COV-2 variants have evolved from the original WT strain of the virus (genome reference sequence: genbank accession NC_045512.2). As described herein, a receptor binding domain may be from a WT, alpha, beta, gamma, kappa, delta, lambda, iota, mu or omicron strain (Table 3) and other strains that are expected to emerge with mutations in the RBD. In any embodiment, the receptor binding domain may be N334-P527 of a WT, alpha, beta, gamma, kappa, delta, lambda, iota, mu, or omicron strains and others that may emerge such as delta plus, for example Y.1, AY.2, AY.3 and AY.4.2. The alpha variant carries 1 mutation in the RBD compared to the original WT strain: N501Y. The beta variant carries three mutations in the RBD compared to the original WT strain: N501Y, E484K and K417N. The gamma variant carries three mutations in the RBD compared to the original WT strain: K417T, E484K and N501Y. The kappa variant carries two mutations in the RBD compared to the original WT strain: L452R and E484Q. The delta variant carries two mutations in the RBD compared to the original WT strain: L452R and T478K. The lambda variant carries two mutations in the RBD compared to the original WT strain: L452Q and F490S. The iota variant carries 1 mutation in the RBD compared to the original WT strain: E484K. The mu variant carries two mutations in the RBD compared to the original WT strain: E484K and N501Y. The omicron variant carries fifteen mutations in the RBD compared to the original WT strain: G339D, S371L, S373P, S375F, K417N, N440K, G446S, S477N, T478K, E484A, Q493R, G496S, Q498R, N501Y and Y505H.

The beta RBD and hence a chimeric or fusion protein of the invention comprising a beta RBD (e.g. SEQ ID NO: 22) (with mutations (N501Y, K417N and E484K) is closer to omicron than a wildtype RBD because it shares with omicron the RBD mutations N501Y and K417N (two important mutations known to influence binding to ACE2 (N501Y) and immune evasion (K417N)). For similar reasons, the beta RBD and hence a chimeric or fusion protein of the invention comprising a beta RBD is closer to Gamma (N501Y, E484K (immune evasion) and K417T), Mu (N501Y and E484K) and lota (E484K). A chimeric or fusion protein of the invention or composition of the invention comprising a beta RBD (e.g. SEQ ID NO: 2) should drive superior responses against Gamma, Mu, lota and Omicron.

TABLE 3
Summary of SARS-CoV-2 variants or strains and RBD mutations

| Nextstrain | | WHO | | RBD mutations compared |
|---|---|---|---|---|
| Clade | Pango Lineage | Label | Other | to original WT strain |
| 20I(Alpha, V1) | B.1.1.7 | α (Alpha) | VOC 202012/01 | N501Y |
| 20H(Beta, V2) | B.1.351 | β (Beta) | 501Y.V2 | K417N, E484K, N501Y |
| 20J(Gamma, V3) | P.1 | γ (Gamma) | | K417T, E484K, N501Y |
| 21A(Delta) | B.1.617.2 | δ (Delta) | | L452R, T478K |
| 21B(Kappa) | B.1.617.1 | κ (Kappa) | | L452R, E484Q |
| 21C(Epsilon) | B.1.427, B.1.429 | ε (Epsilon) | CAL.20C | L452R |
| 21D(Eta) | B.1.525 | η (Eta) | | E484K |
| 21F(Iota) | B.1.526 | ι (Iota) | (Part of Pango lineage) | E484K |
| 21G(Lambda) | C.37 | λ (Lambda) | | L452Q, F490S |
| 21H(Mu) | B.1.621 | μ (Mu) | | E484K, N501Y |
| 21K(Omicron) | B.1.1.529 | ο Omicron | | G339D, S371L, S373P, |
| | | | | S375F, K417N, N440K, |
| | | | | G446S, S477N, T478K, |
| | | | | E484A, Q493R, G496S, |
| | | | | Q498R, N501Y, Y505H |
| 20E (EU1) | B.1.177 | | EU1 | none |
| 20B/S:732A | B.1.1.519 | | | T478K |
| 20A/S:126A | B.1.620 | | | S477N, E484K |
| 20A.EU2 | B.1.160 | | | S477N |
| 20A/S:439K | B.1.258 | | | S439K |
| 20A/S:98F | B.1.221 | | | none |
| 20C/S:80Y | B.1.367 | | | none |
| 20B/S:626S | B.1.1.277 | | | none |
| 20B/S:1122L | B.1.1.302 | | | none |

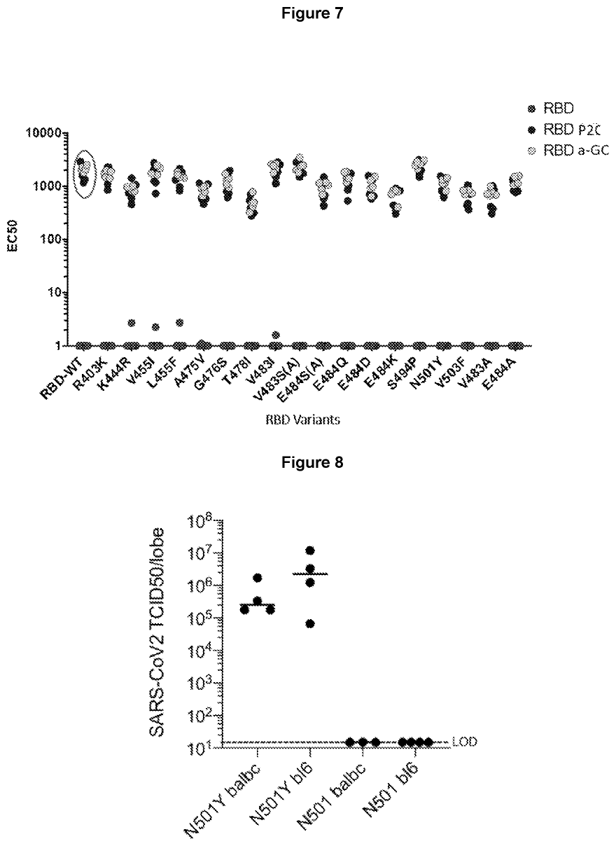

In any aspect, a receptor binding domain from a Spike protein of a coronavirus comprises, consists essentially of or consists of an amino acid sequence of any one or more of SEQ ID NO: 15 or 28, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NO: 15 or 28. In one embodiment, the amino acid sequence may include one or more of the mutations as shown in FIG. 6 or 7, or described herein in relation to a SARS-COV-2 variant, such as an alpha, beta, gamma, kappa or delta strain.

In any aspect, a receptor binding domain from a Spike protein of a coronavirus comprises, consists essentially of or consists of an amino acid sequence of any one or more of SEQ ID NO: 15 or 28 having with 0 to 8 amino acid insertions, deletions, substitutions or additions (or a combination thereof) In some embodiments, the relevant amino acid sequence may have from 0 to 7, preferably from 0 to 6, preferably from 0 to 5, preferably from 0 to 4, preferably from 0 to 3, preferably from 0 to 2, preferably from 0 to 1 amino acid insertions, deletions, substitutions or additions (or a combination thereof), wherein the amino acid insertions, deletions, substitutions or additions (or a combination thereof) are located at the N- and/or C-terminus.

### Fc Region of an Antibody and Fc Receptor Binding Domains

The term “Fc region” herein is used to define a C-terminal region of an immunoglobulin heavy chain that contains at least a portion of the constant region. In other words, the Fc region contains two heavy chain fragments comprising the CH2 and CH3 domains of an antibody. In the context of the present invention, the Fc region comprises two heavy chain fragments, preferably the CH2 and CH3 domains of said heavy chain. The two heavy chain fragments are held together by two or more disulfide bonds and by hydrophobic interactions of the CH3 domains. The heavy chain constant domains that correspond to the different classes of immunoglobulins are called a, 8, &, Y, and u.

In some embodiments, the RBD-Fc dimer does not exhibit any effect function or any detectable effector function. “Effector functions” or “effector activities” refer to those biological activities attributable to the Fc region of an antibody, which vary with the antibody isotype. Examples of antibody effector functions include: C1q binding and complement dependent cytotoxicity (CDC); antibody dependent cell-mediated cytotoxicity (ADCC); phagocytosis; down regulation of cell surface receptors (e.g. B cell receptor); and B cell activation. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity). The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). For example, an antibody variant may comprise an Fc region with one or more amino acid substitutions which diminish FcγR binding, e.g., substitutions at positions 234 and 235 of the Fc region (EU numbering of residues). For example, the substitutions are L234A and L235A (LALA) (See, e.g., WO 2012/130831). Further, alterations may be made in the Fc region that result in altered (i.e., diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

In some aspects, the Fc region includes mutations to the complement (C1q) and/or to Fc gamma receptor (FcγR) binding sites. In some aspects, such mutations can render the fusion protein incapable of antibody directed cytotoxicity (ADCC) and complement directed cytotoxicity (CDC).

The term “Fc region” also includes native sequence Fc regions and variant Fc regions. The Fc region may include the carboxyl-terminus of the heavy chain. Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. Amino acid sequence variants of the Fc region of an antibody may be contemplated. Amino acid sequence variants of an Fc region of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the Fc region of the antibody. Any combination of deletion, insertion, and substitution can be made to arrive at the final construct, provided that the final construct possesses the desired characteristics, e.g., inducing or supporting an anti-inflammatory response.

The Fc region of the antibody may be an Fc region of any of the classes of antibody, such as IgA, IgD, IgE, IgG, and IgM. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. Accordingly, as used in the context of the present invention, the antibody may be an Fc region of an IgG. For example, the Fc region of the antibody may be an Fc region of an IgG1, an IgG2, an IgG2b, an IgG3 or an IgG4. In some aspects, the fusion protein of the present invention comprises an IgG of an Fc region of an antibody. In the context of the present invention, the Fc region of the antibody is an Fc region of an IgG, preferably IgG1.

The Fc region is composed of two heavy chains that contribute two or three constant domains depending on the class of the antibody. By binding to specific proteins, the Fc region ensures that each antibody generates an appropriate immune response for a given antigen.

An Fc receptor binding domain is any protein or polypeptide that binds to the Fc receptor on the surface of a cell. The Fc receptor binding domain may be an antigen binding domain of an antibody. The Fc receptor binding domain also binds to various cell receptors, such as Fc receptors, and other immune molecules, such as complement proteins.

### Linkers

Moreover, the herein provided fusion proteins may comprise a linker (or “spacer”). In the context of the present invention, the 2 or more polypeptides comprising or consisting of an amino acid sequence of a receptor binding domain, or the dimer of receptor binding domains from a spike protein of a coronavirus, is fused via a linker at the C-terminus to the Fc region or Fc receptor binding domain.

A linker is usually a peptide having a length of up to 20 amino acids. The term “linked”, “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 amino acids. For example, the herein provided fusion protein may comprise a linker between the 2 or more polypeptides comprising or consisting of an amino acid sequence of a receptor binding domain, or the dimer of receptor binding domains from a spike protein of a coronavirus, and the Fc region of the antibody, such as between the N-terminus of the Fc regions/FcR binding domains and the C-terminus of the receptor binding domain polypeptide. Such linkers have the advantage that they can make it more likely that the different polypeptides of the fusion protein fold independently and behave as expected.

Thus, in the context of the present invention the 2 or more polypeptides comprising or consisting of an amino acid sequence of a receptor binding domain, or the dimer of receptor binding domains from a spike protein of a coronavirus, and the Fc region of an antibody or Fc receptor binding domain may be comprised in a single covalently associated multi-functional polypeptide.

In some aspects, the fusion protein of the present invention includes a peptide linker. In some aspects, the peptide linker can include the amino acid sequence Gly-Gly-Ser (GGS), Gly-Gly-Gly-Ser (GGGS) or Gly-Gly-Gly-Gly-Ser (GGGGS). In some aspects, the peptide linker can include the amino acid sequence GGGGS (a linker of 6 amino acids in length) or even longer. The linker may a series of repeating glycine and serine residues (GS) of different lengths, i.e., (GS) n where n is any number from 1 to 15 or more. For example, the linker may be (GS) 3 (i.e., GSGSGS) or longer (GS) 11 or longer. It will be appreciated that n can be any number including 1, 2, 3, 4, 5, 6, 7, 8, 9, 10, 11 or more. Fusion proteins having linkers of such length are included within the scope of the present invention. Additionally, fusion proteins that have no linker are included within the scope of the present invention.

### Nucleic Acids

Nucleic acid molecules that encode any of the polypeptides of the invention are also within the scope of the invention. The nucleic acids are useful, for example, in making the polypeptides of the present invention and as therapeutic agents. They may be administered to cells in culture or in vivo and may include a secretory signal that directs or facilitates secretion of the polypeptide of the invention from the cell. Also within the scope of the invention are expression vectors and host cells that contain or include nucleic acids of the invention (described further below). While the nucleic acids of the invention may be referred to as “isolated,” by definition, the polypeptides of the invention are not wild-type polypeptides and, as such, would not be encoded by naturally occurring nucleic acids. Thus, while the polypeptides and nucleic acids of the present invention may be “purified,” “substantially purified,” “isolated,” “recombinant” or “synthetic” they need not be so in order to be distinguished from naturally occurring materials.

An “isolated” nucleic acid molecule is a nucleic acid molecule that is identified and separated from at least one contaminant nucleic acid molecule with which it is ordinarily associated in the natural source of the polypeptide encoding, typically spike protein, nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes nucleic acid molecules contained in infected cells that ordinarily express spike protein where, for example, the nucleic acid molecule is in a chromosomal location different from that of natural cells.

The terms “nucleic acid molecule” and “polynucleotide” are used interchangeably herein and refer to a polymeric form of nucleotides of any length, either deoxyribonucleotides or ribonucleotides or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or purified form. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides of the invention can be synthesised according to methods well known in the art, as described by way of example in Sambrook et al (1989, Molecular Cloning—a laboratory manual; Cold Spring Harbor Press).

The polynucleotide molecules of the present invention may be provided in the form of an expression cassette which includes control sequences operably linked to the inserted sequence, thus allowing for expression of the polypeptide of the invention in vivo in a targeted subject. These expression cassettes, in turn, are typically provided within vectors (e.g., plasmids or recombinant viral vectors) which are suitable for use as reagents for nucleic acid immunization. Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Thus, the present invention provides a vector for use in preventing or treating an inflammatory disease or condition comprising a polynucleotide sequence which encodes a polypeptide of the invention and optionally one or more further polynucleotide sequences which encode different polypeptides as defined herein.

Furthermore, it will be appreciated that the compositions and products of the invention may comprise a mixture of polypeptides and polynucleotides. Accordingly, the invention provides a composition or product as defined herein, wherein in place of any one of the polypeptide is a polynucleotide capable of expressing said polypeptide.

Expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard the inventors refer to Sambrook et al (Molecular Cloning A Laboratory Manual, 2nd ed. (Cold Spring Harbor Laboratory, 1989).

Thus, a polypeptide of the invention may be provided by delivering such a vector to a cell and allowing transcription from the vector to occur. Preferably, a polynucleotide of the invention or for use in the invention in a vector is operably linked to a control sequence which is capable of providing for the expression of the coding sequence by the host cell, i.e. the vector is an expression vector.

“Operably linked” refers to an arrangement of elements wherein the components so described are configured so as to perform their usual function. Thus, a given regulatory sequence, such as a promoter, operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

A number of expression systems have been described in the art, each of which typically consists of a vector containing a gene or nucleotide sequence of interest operably linked to expression control sequences. These control sequences include transcriptional promoter sequences and transcriptional start and termination sequences. The vectors of the invention may be for example, plasmid, virus or phage vectors provided with an origin of replication, optionally a promoter for the expression of the said polynucleotide and optionally a regulator of the promoter. A “plasmid” is a vector in the form of an extra-chromosomal genetic element. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polypeptide.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

A polynucleotide, expression cassette or vector according to the present invention may additionally comprise a signal peptide sequence. The signal peptide sequence is generally inserted in operable linkage with the promoter such that the signal peptide is expressed and facilitates secretion of a polypeptide encoded by coding sequence also in operable linkage with the promoter.

Typically, a signal peptide sequence encodes a peptide of 10 to 30 amino acids for example 15 to 20 amino acids. Often the amino acids are predominantly hydrophobic. In a typical situation, a signal peptide targets a growing polypeptide chain bearing the signal peptide to the endoplasmic reticulum of the expressing cell. The signal peptide is cleaved off in the endoplasmic reticulum, allowing for secretion of the polypeptide via the Golgi apparatus. Thus, a peptide of the invention may be provided to an individual by expression from cells within the individual, and secretion from those cells.

### Immunogenic and Vaccine Compositions

The invention further provides compositions comprising the chimeric or fusion proteins defined herein, and the use of such chimeric or fusion proteins in immunogenic or vaccine compositions in the treatment or prevention of coronavirus infection.

The term “vaccine composition” used herein is defined as composition used to elicit an immune response against an antigen (immunogen) within the composition in order to protect or treat an organism against disease.

As used herein, the terms “immunostimulating composition”, “vaccine composition” and “immunogenic composition” may generally be used interchangeably.

The immunostimulating compositions or vaccines of the invention may suitably include a pharmaceutically acceptable carrier, excipient, diluent, adjuvant, vehicle, buffer or stabiliser in addition to one or more peptides of the invention as the therapeutically or prophylactically active ingredient. Such carriers include, but are not limited to, saline, buffered saline, dextrose, liposomes, water, glycerol, polyethylene glycol, ethanol and combinations thereof.

The immunostimulating compositions or vaccine compositions can be adapted for administration by any appropriate route, for example by the parenteral (including subcutaneous, intramuscular, intravenous or intradermal or by injection into the cerebrospinal fluid), oral (including buccal or sublingual), nasal, topical (including buccal, sublingual or transdermal), vaginal or rectal route. Such compositions can be prepared by any method known in the art of pharmacy, for example by admixing peptides with the carrier(s) or excipient(s) under sterile conditions. Typically, the vaccine composition is adapted for administration by the subcutaneous, intramuscular, intravenous or intradermal route, typically by injection. Alternatively, the vaccine composition may be adapted for oral or nasal administration.

An immunostimulating composition or vaccine composition adapted for parenteral administration may be an aqueous and non-aqueous sterile injection solution which can contain anti-oxidants, buffers, bacteriostats and solutes which render the formulation substantially isotonic with the blood of the intended recipient; and aqueous and non-aqueous sterile suspensions which can include suspending agents and thickening agents. Excipients which can be used for injectable solutions include water, alcohols, polyols, glycerine and vegetable oils, for example. The composition can be presented in unit-dose or multi-dose containers, for example sealed ampoules and vials, and can be stored in a freeze-dried (lyophilized) condition requiring only the addition of the sterile liquid carried, for example water for injections, immediately prior to use. Extemporaneous injection solutions and suspensions can be prepared from sterile powders, granules and tablets.

An immunostimulating or vaccine composition adapted for oral administration, can be presented as discrete units such as capsules or tablets; as powders or granules; as solutions, syrups or suspensions (in aqueous or non-aqueous liquids; or as edible foams or whips; or as emulsions)

Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc.

For the preparation of solutions and syrups, excipients which can be used include for example water, polyols and sugars. For the preparation of suspensions, oils (e.g. vegetable oils) can be used to provide oil-in-water or water in oil suspensions.

An immunostimulating or vaccine composition adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. A suitable composition wherein the carrier is a liquid, for administration as a nasal spray or as nasal drops, may comprise an aqueous or oil solution of the active ingredient.

Compositions adapted for administration by inhalation include fine particle dusts or mists that can be generated by means of various types of metered dose pressurised aerosols, nebulizers or insufflators.

An immunostimulating or vaccine composition adapted for transdermal administration may be presented as a discrete patch intended to remain in intimate contact with the epidermis of the recipient for a prolonged period of time. For example, the active ingredient can be delivered from the patch by iontophoresis as generally described in Pharmaceutical Research. 3(6):318 (1986).

A composition adapted for topical administration may be formulated as an ointment, cream, suspension, lotion, powder, solution, paste, gel, spray, aerosol or oil. For infections of the eye or other external tissues, for example mouth and skin, the composition may be applied as a topical ointment or cream. When formulated in an ointment, the active ingredient can be employed with either a paraffinic or a water-miscible ointment base. Alternatively, the active ingredient can be formulated in a cream with an oil-in-water cream base or a water-in-oil base. A pharmaceutical composition adapted for topical administration to the eye may comprise eye drops wherein the active ingredient is dissolved or suspended in a suitable carrier, especially an aqueous solvent. A pharmaceutical composition adapted for topical administration in the mouth may comprise lozenges, pastilles or mouth washes.

The immunostimulating or vaccine composition may contain preserving agents, solubilising agents, stabilising agents, wetting agents, emulsifiers, sweeteners, colourants, odourants, salts (substances of the present invention can themselves be provided in the form of a pharmaceutically acceptable salt), buffers, coating agents or antioxidants.

The vaccine composition of the invention may also contain one or more other prophylactically or therapeutically active agents in addition to the chimeric or fusion protein as defined herein.

A chimeric or fusion protein for use in the vaccine compositions of the invention may or may not be lyophilised.

### Adjuvants

The vaccine compositions of the invention may also include a pharmaceutically acceptable adjuvant in addition to the peptide(s) as defined herein. Adjuvants are added in order to enhance the immunogenicity of the vaccine composition.

Suitable adjuvants are known in the art and include any one described herein, preferably a TLR2-agonist, more preferably a Pam-2-Cys containing molecule such as PEG-R4-Pam-2-Cys, or preferably a stimulator of NKT cells, more preferably alpha-Galactosylceramide (also referred to herein as “α-GalCer”), alpha-glucosylceramide, alpha-glucosyldiacylglycerol, alpha-galactosyldiacylglycerol, beta-mannosylceramide, and analogues thereof comprising variations in acyl and sphingosine chain lengths, saturation, and variations in polar head group composition.

In some embodiments, the TLR2-agonist can be selected from the group consisting of Pam3CSK4, PEG-R4-Pam-2-Cys, MALP-2, lipoteichoic acid, OspA, Porin, LcrV, lipomannan, Lysophosphatidylserine, Lipophosphoglycan (LPG), Glycophosphatidylinositol (GPI) and Zymosan.

In some embodiments, the adjuvant can be selected from the group consisting of is selected from the group consisting of poly-I: C, CpG, poly-ICLC, 1018 ISS, aluminum salts, Amplivax, AS15, B (C, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59®, AddaVax™, monophosphoryl lipid A, Montanide IMS 1312, Montanide ISA 206, Montanide ISA V, Montanide ISA-51, OK-432, OM-174, OM-197-MP-EC, ONTAK. PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon.

In some embodiments, the adjuvant comprises a metabolizable oil and an emulsifying agent (such as a detergent or surfactant). Preferably, the oil and the emulsifying agent are present in the form of an oil-in-water emulsion having oil droplets substantially all of which are less than 1 micron in diameter. Exemplary metabolizable oils and emulsifying agents are described in U.S. Pat. Nos. 6,299,884 and 6,086,901. In one embodiment, the adjuvant comprises an oil-in-water emulsion. Preferably, the oil is squalene. Preferably, the aqueous phase is a citrate buffer (for example 10 mM at pH 6.5).

In one embodiment, the adjuvant comprises squalene in an oil-in-water emulsion. Preferably, the adjuvant further comprises TWEEN® 80 (polyoxyethylenesorbitan monooleate) and Span® 85 (sorbitan trioleate). The adjuvant may comprise 4.3% squalene, 0.5% TWEEN® 80 (polyoxyethylenesorbitan monooleate), 0.5% Span® 85 (sorbitan trioleate), optionally with 400 pig/ml MTP-PE (N-acetylmuramyl-L-alanyl-D-isoglutaminyl-L-alanine-2-[1,2-dipalmitoyl-sn-glycero-3-3(hydroxyphosphoryl-oxy)]ethylamide)

In one embodiment, the composition comprises 50% vol/vol adjuvant, preferably the adjuvant is MF59®. MF59® is an oil-in-water emulsion of squalene oil. Squalene, a naturally occurring substance found in humans, animals and plants, is highly purified for the vaccine manufacturing process. MF59® adjuvant (MF59C.1) is an oil-in-water emulsion with a squalene internal oil phase and a citrate buffer external aqueous phase. See, e.g., U.S. Pat. Nos. 6,299,884 and 6,086,901; Ott et al. “MF59—Design and Evaluation of a Safe and Potent Adjuvant for Human Vaccines,” Vaccine Design: The Subunit and Adjuvant Approach (Powell, M. F. and Newman, M. J. eds.) Plenum Press, New York, pp. 277-296 (1995). Two nonionic surfactants, sorbitan trioleate and polysorbate 80, serve to stabililize the emulsion. The safety of the MF59® adjuvant has been demonstrated in animals and in humans in combination with a number of antigens. See, e.g., Higgins et al., “MF59 Adjuvant Enhances the Immunogenicity of Influenza Vaccine in Both Young and Old Mice,” Vaccine 14 (6): 478-484 (1996). MF59® is 4.3% squalene, 0.5% TWEEN® 80 (polyoxyethylenesorbitan monooleate), 0.5% Span® 85 (sorbitan trioleate), optionally with 400 μg/ml MTP-PE (N-acetylmuramyl-L-alanyl-D-isoglutaminyl-L-alanine-2-[1,2-dipalmitoyl-sn-glycero-3-3(hydroxyphosphoryl-oxy)]ethylamide). An exemplary composition of MF59 comprises citrate buffer pH 6.5 (10 mM citrate, 140 mM NaCl, 0.02% PS80, pH 6.5) and 39 mg/ml Squalene, 4.7 mg/ml Polysorbate 80, 4.7 mg/ml Sorbitan Trioleate, 2.65 mg/ml Sodium Citrate, 0.17 mg/ml Citric Acid monohydrate.

In any embodiment, the adjuvant is any one described herein, preferably PEG-R4-Pam-2-Cys, alpha-Galactosylceramide (also referred to herein as “α-GalCer”) or MF59®.

### Dosages and Route of Administration

As used herein, the upper respiratory tract (URT) may include the following regions: nose and nasal passages, paranasal sinuses, the pharynx, and the portion of the larynx above the vocal folds (cords).

Typically, the lower respiratory tract (LRT) includes the following regions: portion of the larynx below the vocal folds, trachea, bronchi and bronchioles. The lungs can be included in the lower respiratory tract and include the respiratory bronchioles, alveolar ducts, alveolar sacs, and alveoli. In any aspect of the present invention, administration to the URT may be administration to the nose and nasal passages, paranasal sinuses, the pharynx, and the portion of the larynx above the vocal folds (cords). Also contemplated is administration to any one or more regions of the URT provided that the compound is retained in the URT or does not contact a region of the LRT.

The phrase “therapeutically effective amount” generally refers to an amount of one or more polypeptides or polynucleotides of the invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g., side effects, are sometimes manifested along with the desired therapeutic effect; hence, a practitioner balances the potential benefits against the potential risks in determining what is an appropriate “effective amount”.

In some embodiments, an effective amount of a chimeric or fusion protein of the invention for a human subject lies in the range of about 0.25 nmoles/kg body weight/dose to 0.0001 nmoles/kg body weight/dose. Preferably, the range is about 0.25 nmoles/kg body weight/dose to 0.0001 nmoles/kg body weight/dose. More preferably, the range is about 0.002 nmoles/kg body weight/dose to 0.001 nmoles/kg body weight/dose. In some embodiments, the body weight/dose range is about 0.25 nmoles/kg, to 0.001 nmoles/kg, about 0.1 nmoles/kg to 0.001 nmoles/kg, about 0.025 nmoles/kg to 0.001 nmol/kg, about 0.01 nmoles/kg to 0.001 nmoles/kg, or about 0.005 nmoles/kg to 0.001 nmoles/kg body weight/dose. In some embodiments, the amount is at, or about, 0.25 nmoles, 0.1 nmoles, 0.05 nmoles, 0.01 nmoles, 0.005 nmoles, 0.002 nmoles, or 0.001 nmoles/kg body weight/dose of the chimeric or fusion protein of the invention. Dosage regimes are adjusted to suit the exigencies of the situation and may be adjusted to produce the optimum therapeutic dose.

In some embodiments, an effective amount of a chimeric or fusion protein of the invention for a human subject lies in the range of about 1 μg to 100 μg per dose. Preferably, the range is about 1 μg to 50 μg per dose. More preferably, the range is about 1 μg to 20 μg per dose. In some embodiments, the dose is about 5 μg to 100 μg, about 5 μg to 50 μg, about 10 μg to 45 μg, about 10 μg to 25 μg or about 10 μg to 20 μg per dose. In some embodiments, the dose is at, or about, 0.1 μg, 0.2 μg, 0.5 μg, 1 μg, 5 μg, 10 μg, 15 μg, 25 μg, 30 μg, 35 μg, 40 μg, 45 μg, 50 μg. Preferably the dose is 15 μg.

The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. In one aspect, the dose administered to a subject is any dose that reduces viral load.

The chimeric or fusion protein or vaccine compositions thereof can be administered using immunization schemes known by persons of ordinary skill in the art to induce protective immune responses. These include a single immunization or multiple immunizations in a prime-boost strategy. A boosting immunization can be administered at a time after the initial, prime, immunization that is days, weeks, months or even years after the prime immunization. In certain embodiments, a boost immunization is administered 2 weeks, 1 month, 2 months, 3 months, 4 months, 5 months, or 6 months or more after the initial prime immunization. Additionally multiple boost immunizations can be administered weekly, every other week, monthly, every other month, every third month, or more. In other embodiments, the boost immunization is administered every 3 weeks, every 4 weeks, every 5 weeks, every 6 weeks, every 7 weeks, every 8 weeks, every 9 weeks, every 10 weeks, every 11 weeks, or every 12 weeks. In certain embodiments, boosting immunization can continue until a protective anti-SARS-COV-2 antibody titer is seen in the subject's serum. In certain embodiments, a subject is given one boost immunization, two boost immunizations, three boost immunizations, or four or more boost immunizations, as needed to obtain a protective antibody titer. In further embodiments, the adjuvant in the in the initial prime immunization and the adjuvant in the boost immunization are different. Alternatively, the adjuvant in the prime immunization and the adjuvant in the boost immunization are the same.

This dosage can be repeated as often as appropriate. For example, an initial dose of the vaccine may be administered and then a booster administered at a later date.

The composition of the invention can be administered by any convenient route as described herein, such as via the intramuscular, intranasal, subcutaneous, intravenous, intraperitoneal or oral routes.

The composition of the invention is formulated for or adapted for administration by any convenient route as described herein, such as via the intramuscular, intranasal, subcutaneous, intravenous, intraperitoneal or oral routes.

The composition of the invention may be formulated for administration to the URT only. Limitation to the URT may be achieved by an amount, particularly volume and composition of form i.e. particle size, physical form whether dry powder or solution droplet, of composition that would otherwise be administered to the LRT or TRT. Alternatively, the vaccine composition of the invention may be administered via a device that ensures retention in the URT only.

The composition as described herein may be formulated for intranasal administration, including dry powder, sprays, mists, or aerosols. This may be particularly preferred for treatment of coronavirus infection.

There are a number of options that can be employed to limit delivery to the URT using intranasal delivery: (1) Ensuring the droplet/particle size is sufficiently large to prevent access into the LRT (>10 um); (2) For liquids limiting dose volume to minimise run-off/drainage; (3) Similarly administration with the head in an inverted position also minimises run-off/drainage; (4) Inclusion of a viscosity enhancer/mucoadhesive to promote retention in the nasal cavity and prevent run-off/drainage; (5) Use a nasal device that entirely eliminates the potential for LRT exposure e.g. the Optinose bi-directional delivery device. One or a combination of these methods can be applied.

The selection of appropriate carriers depends upon the particular type of administration that is contemplated. For administration via the upper respiratory tract, e.g., the nasal mucosal surfaces, the compound can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or upper respiratory administration.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odour for the formulation. For nasal administration of solutions or suspensions according to the invention, various devices are available in the art for the generation of drops, droplets and sprays. For example, a vaccine composition described herein can be administered into the nasal passages by means of a simple dropper (or pipet) that includes a glass, plastic or metal dispensing tube from which the contents are expelled drop by drop by means of air pressure provided by a manually powered pump, e.g., a flexible rubber bulb, attached to one end.

The tear secretions of the eye drain from the orbit into the nasal passages, thus, if desirable, a suitable pharmaceutically acceptable ophthalmic solution can be readily provided by the ordinary artisan as a carrier for the vaccine composition described herein to be delivered and can be administered to the orbit of the eye in the form of eye drops to provide for both ophthalmic and intranasal administration.

In one embodiment, a premeasured unit dosage dispenser that includes a dropper or spray device containing a solution or suspension for delivery as drops or as a spray is prepared containing one or more doses of the drug to be administered. The invention also includes a kit containing one or more unit dehydrated doses of vaccine composition, together with any required salts and/or buffer agents, preservatives, colorants and the like, ready for preparation of a solution or suspension by the addition of a suitable amount of water. The water may be sterile or nonsterile, although sterile water is generally preferred.

The composition of the invention can be provided in unit dosage form, will generally be provided in a sealed container and may be provided as part of a kit. Such a kit would normally (although not necessarily) include instructions for use. It can include a plurality of said unit dosage forms.

### Methods of Prevention and Treatment

In any aspect, the chimeric or fusion protein of the invention, or composition of the invention generates antibodies to, preferably neutralising antibodies, any one or more of the coronavirus strains described herein, particularly those in Table 3. In any aspect, the chimeric or fusion protein of the invention, or composition of the invention provides a therapeutic or prophylactic treatment for any or more of the coronavirus strains described herein, particularly those in Table 3. In the aspects and embodiments defined below, reference to “a coronavirus” also includes reference to one or more strains of coronavirus as described herein, including but not limited to those in Table 3, for example: WT, alpha and beta strains; WT, alpha, beta and VIC2089 strains; WT, alpha, beta, and delta strains; WT, alpha, beta, delta, delta plus, gamma, lambda, mu, iota, kappa and omicron strains. The chimeric or fusion protein of the invention, or composition of the invention may provide a therapeutic or prophylactic benefit against SARS-COV-2 strains that are different from which the RBD amino acid sequence in the chimeric or fusion protein was derived.

Exemplary strains against which the chimeric or fusion protein of the invention, or composition of the invention provides a therapeutic or prophylactic benefit are shown in the Examples.

In one aspect, the present invention provides a method of treating and/or preventing a disease associated with a coronavirus, the method comprising administering to the subject in need thereof, a chimeric or fusion protein, composition, vaccine or immune stimulating composition as described herein, thereby treating and/or preventing a disease associated with a coronavirus.

In another aspect, the present invention provides a method of treating and/or preventing a disease associated with, or caused by, a coronavirus, the method comprising administering to a subject in need thereof a chimeric or fusion protein, composition, vaccine or immune stimulating composition as described herein described herein, thereby treating and/or preventing a disease associated with, or caused by, a coronavirus.

In another aspect, the present invention provides a method of treating and/or preventing a respiratory disease or condition associated with a coronavirus infection, the method comprising administering to a subject in need thereof a chimeric or fusion protein, composition, vaccine or immune stimulating composition as described herein, thereby treating and/or preventing a respiratory disease or condition associated with a coronavirus infection.

In another aspect, the present invention provides a method for reducing airway inflammation associated with, or caused by, a coronavirus, the method comprising administering to a subject in need thereof a chimeric or fusion protein, composition, vaccine or immune stimulating composition as described herein, thereby reducing airway inflammation associated with, or caused by, a coronavirus.

In another aspect, the present invention also provides a method of improving the ability of a subject to control a respiratory disease or condition during a coronavirus infection, the method comprising administering to a subject in need thereof a chimeric or fusion protein, composition, vaccine or immune stimulating as described herein, thereby improving the ability of a subject to control a respiratory disease or condition during a coronavirus infection.

In another aspect, the present invention provides for use of a chimeric or fusion protein, composition, vaccine or immune stimulating composition as described herein, in the preparation of a medicament for raising an innate immune response in a subject diagnosed with, or suspected of having, a coronavirus infection.

In another aspect, the present invention provides for use of a chimeric or fusion protein, composition, vaccine or immune stimulating composition as described herein, in the preparation of a medicament for treating and/or preventing a disease caused by a coronavirus.

In another aspect, the present invention further provides for use of a chimeric or fusion protein, composition, vaccine or immune stimulating composition as described herein, in the preparation of a medicament for treating and/or preventing a respiratory disease or condition associated with a coronavirus infection in a subject.

In another aspect, the present invention further provides for use of a chimeric or fusion protein, composition, vaccine or immune stimulating composition as described herein, in the preparation of a medicament for treating and/or preventing a coronavirus infection in a subject.

In another aspect, the present invention further provides use of a chimeric or fusion protein, composition, vaccine or immune stimulating composition as described herein, in the preparation of a medicament for reducing airway inflammation in a subject diagnosed with, or suspected of having, a coronavirus infection.

In another aspect, the present invention further provides use of a chimeric or fusion protein, composition, vaccine or immune stimulating composition as described herein, in the preparation of a medicament for improving the ability of a subject to control a respiratory disease or condition during a coronavirus infection.

In one aspect, the present invention provides for a chimeric or fusion protein, composition, vaccine or immune stimulating composition as described herein, for use in raising an innate immune response in a subject diagnosed with, or suspected of having, a coronavirus infection.

In another aspect, the present invention provides for a chimeric or fusion protein, composition, vaccine or immune stimulating composition as described herein, for use in treating and/or preventing a disease caused by a coronavirus in a subject.

In another aspect, the present invention provides for a chimeric or fusion protein, composition, vaccine or immune stimulating composition as described herein, for use in treating and/or preventing a respiratory disease or condition associated with a coronavirus infection in a subject.

In another aspect, the invention provides a chimeric or fusion protein, composition, vaccine or immune stimulating composition as described herein, for use in reducing airway inflammation in a subject diagnosed with, or suspected of having, a coronavirus infection.

In another aspect, the invention provides a chimeric or fusion protein, composition, vaccine or immune stimulating composition as described herein, for use in controlling a respiratory disease or condition during a coronavirus infection in a subject.

In any aspect of the invention, where prevention or prophylaxis is intended or required, chimeric or fusion protein, composition, vaccine or immune stimulating composition as described herein is administered to the subject before any clinically or biochemically detectable symptoms of viral infection.

In any aspect of the invention, administration of a chimeric or fusion protein, composition, vaccine or immune stimulating composition as described herein to a subject reduces viral load in the subject. Preferably, the viral load is reduced in the respiratory tract, for example the upper and/or lower respiratory tract. Preferably, the viral load is reduced in the nasal cavity and pharynx (i.e. throat). In alternative embodiments, the viral load may be in the gastrointestinal tract, in the peripheral circulation, in the heart, liver, kidney, spleen or other organ known to be susceptible to infection with coronavirus, preferably to infection with SARS-COV-2.

The term “coronavirus infection” or “CoV infection” as used herein, refers to infection with a coronavirus such as SARS-COV-2, MERS-COV, or SARS-COV. The term includes coronavirus respiratory tract infections, often in the lower respiratory tract. Symptoms can include high fever, dry cough, shortness of breath, pneumonia, gastro-intestinal symptoms such as diarrhoea, organ failure (kidney failure and renal dysfunction), septic shock, and death in severe cases.

A reduction in coronavirus infection may be determined using any method known in the art or described herein, including measuring viral load in a sample from the subject after treatment and comparing it to viral load in a sample from the same subject before treatment. The sample may be any biological sample obtained from the subject, and may include blood, saliva, urine, faeces, nasal wash, sputum, and mucous secretions. The sample may be taken from the respiratory tract, preferably the upper respiratory tract, for example the nose or pharynx (i.e. throat). The term ‘respiratory disease’ or ‘respiratory condition’ refers to any one of several ailments that involve inflammation and affect a component of the respiratory system including the upper (including the nasal cavity, pharynx and larynx) and lower respiratory tract (including trachea, bronchi and lungs). The inflammation in the upper and lower respiratory tract may be associated with or caused by viral infection.

A symptom of respiratory disease may include cough, excess sputum production, a sense of breathlessness or chest tightness with audible wheeze.

The existence of, improvement in, treatment of or prevention of a respiratory disease may be determined by any clinically or biochemically relevant method of the subject or a biopsy therefrom. For example, a parameter measured may be the presence or degree of lung function, signs and symptoms of obstruction; exercise tolerance; night time awakenings; days lost to school or work; bronchodilator usage; Inhaled corticosteroid (ICS) dose; oral glucocorticoid (GC) usage; need for other medications; need for medical treatment; hospital admission.

As used herein, the term respiratory infection means an infection anywhere in the respiratory tract. Examples of respiratory infection include but are not limited to colds, sinusitis, throat infection, tonsillitis, laryngitis, bronchitis, pneumonia, or bronchiolitis. Preferably, in any embodiment of the invention the respiratory infection is a cold. An individual may be identified as having a respiratory tract infection by viral testing and may exhibit systems of itchy watery eyes, nasal discharge, nasal congestion, sneezing, sore throat, cough, headache, fever, malaise, nausea, vomiting, fatigue and weakness. In one aspect, a subject having a respiratory infection may not have any other respiratory condition. Detection of the presence or amount of virus, preferably coronavirus, may be by PCR/sequencing of RNA isolated from clinical samples (nasal wash, sputum, BAL) or serology.

As used herein, the term respiratory infection means an infection by a coronavirus, preferably by SARS-COV-2, anywhere in the respiratory tract.

An individual may be identified as having a respiratory tract infection by viral testing and may exhibit symptoms of itchy watery eyes, nasal discharge, nasal congestion, sneezing, sore throat, cough, headache, fever, malaise, nausea, vomiting, fatigue and weakness. In one aspect, a subject having a respiratory infection may not have any other respiratory condition. Detection of the presence or amount of virus may be by PCR/sequencing of RNA isolated from clinical samples (nasal wash, sputum, BAL) or serology. The terms “treatment” or “treating” of a subject includes the application or administration of a chimeric or fusion protein, composition, vaccine or immune stimulating composition of the invention to a subject (or application or administration of a chimeric or fusion protein, composition, vaccine or immune stimulating composition as described herein of the invention to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

Given the breadth of symptoms caused by coronaviruses, including by SARS-CoV-2, it will be appreciated that a positive response to vaccination according to the methods described herein, may include any amelioration or improvement of symptoms experienced by the subject.

For example, a positive response to vaccination may be a reduction in general levels of fatigue, muscle pain, headache and/or lethargy in the subject. A positive response may also include a reduction in fever, and a return to afebrile state in the subject.

A positive response to vaccination may also be prevention or attenuation of worsening of respiratory symptoms following a respiratory virus infection. This could be assessed by comparison of the mean change in disease score from baseline to end of study period, for example, based on a questionnaire, and could also assess lower respiratory symptom score (LRSS-symptoms of chest tightness, wheeze, shortness or breath and cough) daily following infection/onset of cold symptoms. Change from baseline lung function (peak expiratory flow PEF) could also be assessed and a positive response to therapy could be a significant attenuation in reduced PEF. For example, a placebo treated group would show a significant reduction in morning PEF of 15% at the peak of exacerbation whilst the treatment group would show a non-significant reduction in PEF less than 15% change from baseline.

A positive response to vaccination may also be a reduction in the presence of ground-glass type opacities in the lung periphery or near the pleura (for example, as determined using chest CT imaging techniques).

A positive response to vaccination may also include an increase or return to normal levels of blood oxygenation levels.

A positive response to vaccination may also include an improvement in cardiovascular disorders such as alterations in blood pressure and increased presence of clotting factors.

Protective immune responses can include humoral immune responses and cellular immune responses. Protection against SARS-COV-2 is believed to be conferred through serum neutralizing antibodies (humoral immune response) directed to the spike protein, with mucosal IgA antibodies and cell-mediated immune responses also playing a role. Cellular immune responses are useful in protection against SARS-COV-2 infection with CD4+ and CD8+ T cell responses and memory B cell responses being particularly important. CD8+ immunity is of particular importance in killing virally infected cells. Natural killer cells and NKT cells may also be important for killing and/or clearance of virally infected cells.

### Kits

In another embodiment there is provided a kit or article of manufacture including one or more proteins, polypeptides or polynucleotides of the invention and/or immunogenic composition as described above.

In yet another aspect, the present invention provides a kit of parts comprising a vaccine composition of the invention and one or more adjuvants for separate, subsequent or simultaneous administration to a subject.

In other embodiments there is provided a kit for use in a therapeutic or prophylactic application mentioned above, the kit including:
a container holding a protein, polypeptide, polynucleotide or immunogenic composition of the invention;
a label or package insert with instructions for use.

In any embodiment the kit may contain one or more further active principles or ingredients for eliciting an immune response to coronavirus in a subject.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use and indicates that the therapeutic or prophylactic composition can be used to treat an inflammatory disease or condition described herein.

The kit may comprise (a) a therapeutic or prophylactic composition; and (b) a second container with a second active principle or ingredient contained therein. The kit in this embodiment of the invention may further comprise a package insert indicating the composition and other active principle can be used to treat a disorder or prevent a complication stemming from an inflammatory disease or condition described herein. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In any embodiment the therapeutic composition may be provided in the form of a device, disposable or reusable, including a receptacle for holding the therapeutic, prophylactic or immunogenic composition. In one embodiment, the device is a syringe. The device may hold 1-2 mL of the therapeutic or immunogenic composition. The therapeutic or prophylactic composition may be provided in the device in a state that is ready for use or in a state requiring mixing or addition of further components.

It will be understood that the invention disclosed and defined in this specification extends to all alternative combinations of two or more of the individual features mentioned or evident from the text or drawings. All of these different combinations constitute various alternative aspects of the invention.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1—Generation of RBD-IgG1 Fc Fusion Proteins

### Materials and Methods

### RBD-Mouse IgG1 Fc-Fusion:

To produce recombinant RBD-mouse IgG1 Fc-fusion protein (SEQ ID NO: 3), recombinant DNA fragments encoding the truncated receptor binding domain (RBD) of ancestral SARS-COV-2 (N334-P527; GenBank accession NC_045512.2; SEQ ID NO: 15) were synthesised between a 5′ AgeI and a 3′ BamHI cloning site (GeneArt Gene Strings, Thermo Scientific). These were then cloned into the mammalian expression vector pHLSec containing mouse IgG1 Fc domain (SEQ ID NO: 16) from the core hinge region through to the C-terminal lysine and including an N-terminal GSGSG linker (SEQ ID NO: 17), thereby fusing the RBD to mIgG1-Fc.

### RBD-Human IgG1 Fc-Fusion:

To produce recombinant RBD-human IgG1 Fc-fusion protein (SEQ ID NO: 25), recombinant DNA fragments encoding the truncated receptor binding domain (RBD) of ancestral SARS-COV-2 (N334-P527; GenBank accession NC_045512.2; SEQ ID NO: 15) fused via a GSGSG linker (SEQ ID NO: 17) to the Fc domain of human IgG1 (SEQ ID NO: 18) from the core-hinge region to the C-terminal lysine followed by a stop codon were codon-optimised for mammalian expression (GeneArt Gene Strings, Thermo Scientific) and synthesised between a 5′ NheI and a 3′ XhoI cloning site (IDT). This was then cloned into the mammalian expression vector pHLSec (SEQ ID NO: 4).

### RBD Monomer:

To produce recombinant RBD monomer, recombinant DNA fragments encoding the truncated receptor binding domain (RBD) of ancestral SARS-COV-2 (N334-P527; GenBank accession NC_045512.2; SEQ ID NO: 15) were codon-optimised for mammalian expression (GeneArt Gene Strings, Thermo Scientific) and synthesised between a 5′ NheI and a 3′ BamHI cloning site (IDT). This were then cloned into the mammalian expression vector pHLSec to incorporate a 6-HIS tag followed by a stop codon at the C-terminus (SEQ ID NO: 8). The RBD tandem dimer is also referred to herein as the single chain RBD dimer.

### RBD Tandem Dimer:

To produce recombinant RBD-tandem dimer based on Dai et al., (Cell, 2020), recombinant DNA fragments encoding 2 truncated receptor binding domains (RBD) of ancestral SARS-COV-2 (R319-K537; genbank accession NC_045512.2, SEQ ID Nos: 19 and 20) repeated in tandem were codon-optimised for mammalian expression (GeneArt Gene Strings, Thermo Scientific) and synthesised between a 5′ NheI and a 3′ BamHI cloning site (IDT). This were then cloned into the mammalian expression vector pHLSec to incorporate a 6-HIS tag followed by a stop codon at the C-terminus (SEQ ID NO: 11).

### RBD-Beta Human IgG1 Fc Fusion

To enable the cloning of the recombinant DNA fragments encoding the truncated RBD of ancestral SARS-COV-2 (N334-P527; GenBank accession NC_045512; SEQ ID NO: 15) fused via a GSGSG linker (SEQ ID NO: 17) to the Fc domain of human IgG1 (SEQ ID NO: 18) from the core-hinge region to the C-terminal lysine followed by a stop codon from the mammalian expression vector pHLSec into the mammalian expression vector pXC-17.4 PCR primers were designed. The resulting PCR product was cloned into the mammalian expression vector pXC-17.4 using the restriction enzymes sites HindIII and EcoRI to produce the polynucleotide as set forth in SEQ ID NO: 29. To introduce the mutations K417N, E484K and N501Y PCR mutagenesis was used. This was the construct used for production of the DoCo-Pro-RBD-1 ADP.

### Protein Expressions:

All RBD proteins, with the exception of the RBD-beta human IgG1 Fc fusion protein, were expressed by transient transfection of Expi293S cells using ExpiFectamine 293 Transfection Kits as per manufacturer's instruction (ThermoFisher Scientific). Proteins were harvested on day six. RBD-Fc proteins were purified from supernatants by Protein A Sepharose (CL-4B, Cytiva) and RBD monomers and RBD tandem dimers were purified by Ni-NTA resin (HisPur, Thermo Scientific). All proteins were further purified by gel filtration size exclusion chromatography: RBD-Fc and RBD tandem dimers using a Superdex-200 column (Cytiva), and RBD monomers using a Superdex S75 column (Cytiva). All proteins were sterile filtered and stored at −80° C. prior to use.

A research cell bank (RCB) for a stable pool expressing the beta RBD-hFc protein was developed at the National Biologics Facility (QLD node). Cells from a single vial were revived and expanded in shake flasks and the protein produced in a fed batch shaker flask production over 12 days according to the Lonza GSv9™ recommendations. The conditioned media was harvested by depth filtration and 0.2 μm filtration. The antigen was captured on a protein A resin, MabSelect PrismA (Cytiva) with elution at pH 4.0. The antigen was subjected to low pH viral inactivation, with acidification to pH 3.5, holding for >60 min, then neutralisation to pH 5.5. The antigen was buffer exchanged to pH 6.5 citrate buffer and formulated with PS80 to 0.02% and diluted to 1.06 mg/ml before being aseptically filled to vials.

### AF647-Conjugation of Proteins:

Purified RBD-mFc as well as mouse IgG1 isotype control (clone MOPC-21, Biolegend) were then labelled with Alexa Fluor 647 antibody labelling kit (Thermo Scientific) as per manufacturer's instruction.

### Results and Discussion

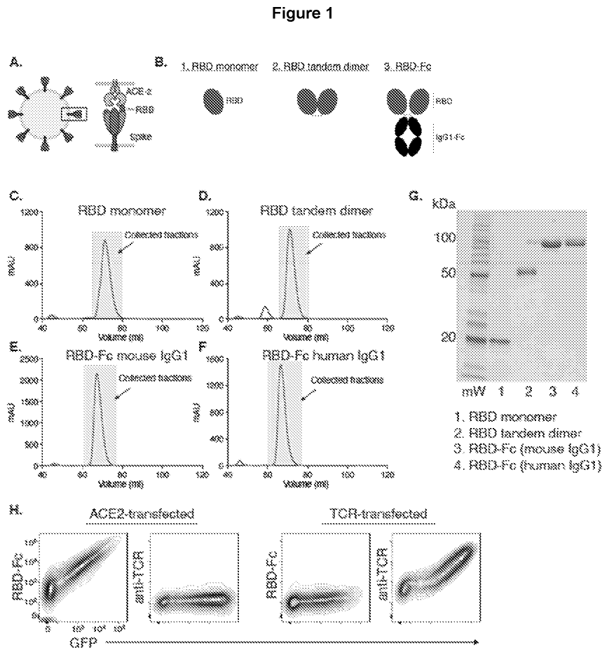

DNA encoding the SARS-COV-2 spike receptor binding domain (RBD) (FIG. 1A) in an isolated form was cloned into the mammalian expression vector pHLSec for expression in three different forms: RBD monomer, RBD tandem dimer and RBD-IgG1 Fc dimer (FIG. 1B). Both mouse and human RBD-IgG1 Fc dimers were produced. These proteins were expressed by transiently transfecting mammalian Expi-293S cells and, 6 days later, proteins isolated and purified by gel filtration size exclusion chromatography (FIG. 1C-G) prior to being sterile filtered and stored at −80° C.

The activity of the purified RBD-Fc dimeric proteins was validated by staining 293T cells transfected to express human ACE2, the natural receptor for the RBD (FIG. 1H). A control cell line transfected with an irrelevant protein (T cell receptor) did not stain with RBD-Fc dimer but did stain with anti-TCR antibody as expected.

### Example 2—Comparison of Subcutaneous and Intranasal Routes of Inoculation of RBD-Fc Dimer with and without Adjuvant

### Materials and Methods

### Vaccination and Bleeding Regime

BALB/c mice (n=5 per group) were vaccinated either via the subcutaneous route at the base of the tail (100 μl) or intranasally to the URT (15 μl) with 0.1 nmoles of RBD dimer administered without adjuvant or administered in the presence of 0.3 nmoles PEG-R4-Pam-2-Cys or 0.2 μg a-GC adjuvant. Mice were primed on day 0 and boosted on day 28. Mice were bled just prior to the first injection (pre-bleed), just prior to the second injection (1°), and two weeks following the second injection (2°).

### Enzyme-Linked Immunosorbent Assay (ELISA) for Measurement of RBD-Specific Antibody Responses

RBD-specific antibody titres were determined by ELISA. Flat bottom 96 well maxisorp plates (ThermoFisher Scientific) were coated with 50 μl/well of RBD monomer at a concentration of 2 μg/ml in Dulbecco's phosphate buffered saline (DPBS; Gibco Life Technologies). Plates were incubated overnight at 4° C. in a humidified atmosphere. Unbound antibody was removed, and wells were blocked with 100 μl/well of 1% bovine serum albumin (BSA fraction V, Invitrogen Corporation, Gibco) in PBS for 1-2 hours before washing 3 times with PBS containing 0.05% v/v Tween-20 (PBST). Serial dilutions of mouse sera were added to wells and left to incubate overnight at room temperature. After washing, bound Ab was detected using horseradish peroxidase (HRP)-conjugated rabbit anti-mouse Ig Abs (Dako, Denmark). The detection antibody was incubated for 1 hour at room temperature in a humidified atmosphere and the plates then washed five times with PBST. 100 μl of tetramethylbenzidine substrate (TMB, BD Biosciences, cat #555214) was then added to each well and the reaction was stopped after 5-7 minutes by the addition of 100 μl/well of 1M orthophosphoric acid (BDH Chemicals, Australia). A Labsystems Multiskan microplate reader (Labsystems, Finland) was used to measure the optical density (OD) of each well at wavelengths of 450 nm and 540 nm. The titres of Ab are expressed as the reciprocal of the highest dilution of serum required to achieve an OD of 0.3.

### Microneutralisation Assay

SARS-COV-2 isolates used in the microneutralisation assay were propagated in Vero cell cultures and stored at −80° C. Flat-bottom 96-well plates were seeded with Vero cells at 2×10⁴ cells/well the day before assay. Serial 2-fold dilutions of heat-inactivated sera were incubated with 100 TCID₅₀ (50% tissue culture infectious dose) of SARS-COV-2 for 1 hour and residual virus infectivity was assessed in quadruplicate wells of Vero cells. Plates were incubated at 37° C. and viral cytopathic effect was read on day 5. The neutralising antibody titre was calculated using the Reed/Muench method.

### SARS-COV-2 Surrogate Virus Neutralisation Test

The detection of circulating antibodies directed against the spike protein receptor binding domain (RBD) based on antibody-mediated blockage of interaction between the ACE2 receptor protein and RBD was measured in the mouse serum using SARS-COV-2 Surrogate Virus Neutralisation Test (GenScript, USA) according to the manufacturer's instructions. In brief, 10 μl of mouse serum was diluted with 90 μl of sample dilution buffer and incubated with horseradish peroxidase conjugated SARS-CoV-2 RBD protein (HRP-RBD); the test solution was added to wells coated with fixed ACE2 receptor. The degree to which serum inhibited binding of the HRP-RBD to ACE2 receptors, compared to control serum, was determined by optical density reading, with 20% inhibition and above considered a positive result.

### Results and Discussion

Groups of 5 BALB/c mice were immunised with RBD-mouse IgG1-Fc dimer (RBD), either without adjuvant, or mixed with PEG-R4-Pam-2-Cys (P2C) adjuvant or α-galactosylceramide (a-GC) adjuvant. Two immunisation routes were tested: Subcutaneous and Intranasal (upper respiratory tract, URT).

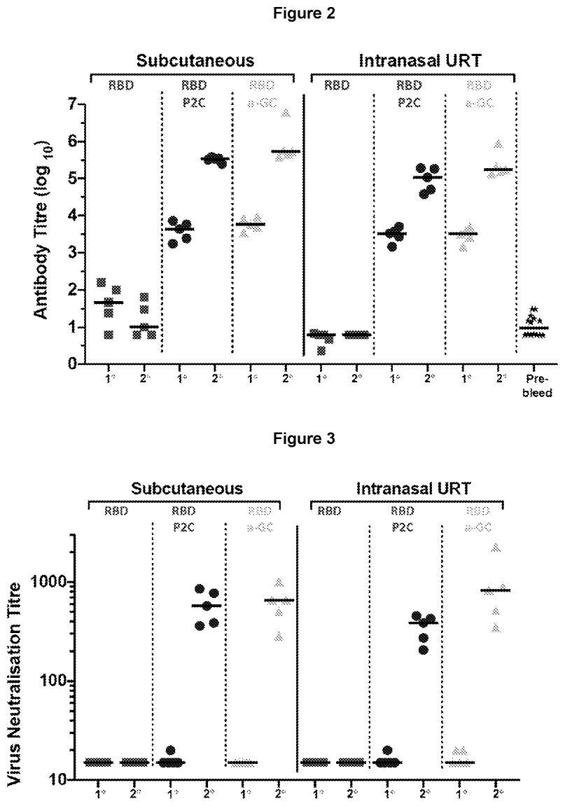

The first injection (day 0) was followed four weeks later by an identical boost injection. Mice were bled just prior to the first injection (pre-bleed), just prior to the second injection (1°), and two weeks following the second injection (2°). Total anti-RBD IgG antibody titres were determined using an RBD specific ELISA assay for each mouse in each group (FIG. 2).

These results showed that RBD without adjuvant barely induced a response above background determined by the pre-bleed samples. However, both adjuvants (P2C and a-GC) induced a very strong antibody titre (between 10³ and 10⁴) after the primary injection and >105 antibody titre after the secondary injection.

These serum samples were also tested in a microneutralisation assay using the SARS-COV-2 virus, where neutralisation titre was determined by the titre required to inhibit infection of Vero cells. This assay demonstrated that despite a high antibody titre after the primary immunisation, very little or no antibody was induced. However, after the secondary immunisation, a very strong neutralising antibody response was detected in all groups that received RBD plus adjuvant, ranging between 200 and 2000 antibody titre (FIG. 3).

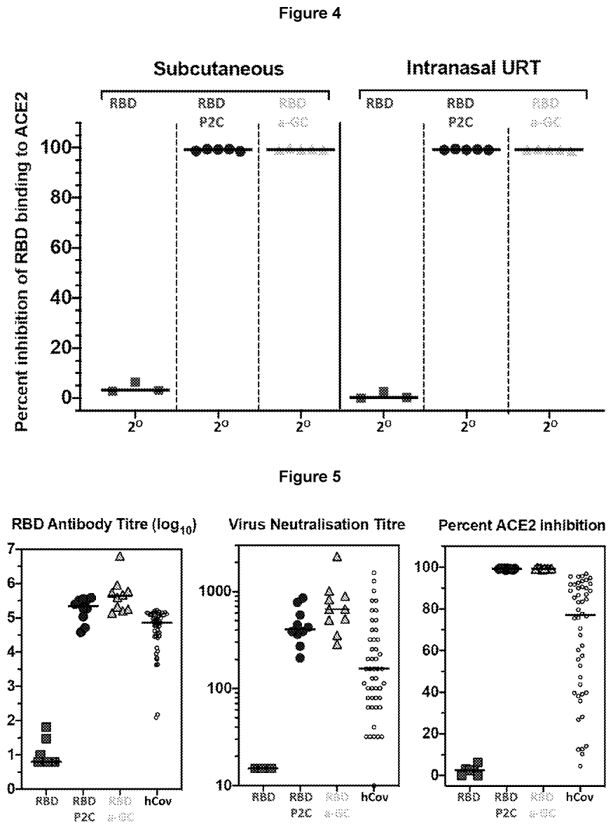

As an independent validation of the neutralising potential of these serum samples, a distinct assay known as the surrogate virus neutralisation test (sVNT) (Tan et al. (2020) Nature Biotechnology) was also employed. This test measures the ability of an antibody-containing sample to interfere with RBD binding to ACE2. Due to the limited numbers of tests that could be performed, only the secondary serum samples were tested and these revealed that the adjuvanted RBD vaccine provided >95% inhibition of RBD-ACE-2 binding in this assay. Sera from mice immunised with non-adjuvanted RBD vaccine failed to register a neutralising antibody response in this assay (FIG. 4).

These data are also presented in association with similar data derived from human convalescent patient samples (hCov) collected and assayed over the same time frame, although not run in the same experiments (FIG. 5). These data are provided as a comparison only.

### Example 3—Testing of Spike Protein Variants

Human Convalescent Plasma Samples (hCov)

Human convalescent plasma samples (hCov) DD1, DD2, DD3 and DD4 were sourced from Professor Katherine Kedzierska (Department of Microbiology and Immunology, The Peter Doherty Institute for Infection and Immunity). These patients were infected in April 2020 with the SARS-COV-2 Wuhan Index strain VIC01. Sera were tested for neutralising activity against 501Y and 477N mutants (VIC2089 and VIC4881) and the original VIC01 strain using the microneutralisation assay.

### Mouse Serum Samples

BALB/c mice (n=5 per group) were vaccinated via the subcutaneous route at the base of the tail with 0.1 nmoles of RBD dimer administered without adjuvant or administered in the presence of 0.3 nmoles PEG-R4-Pam-2-Cys or 0.2 μg a-GC adjuvant. Mice were primed on day 0 and boosted on day 28. Sera collected 4 weeks after the second dose were tested for neutralising activity against 501Y and 477N mutants (VIC2089 and VIC4881) and the original VIC01 strain using the microneutralisation assay. These sera were also tested for binding to 18 RBD variant proteins (GenScript, USA) using a multiplex bead array.

### Results and Discussion

As two SARS-COV-2 variants had emerged in Australia (D614G/N501Y) and (D614G/S477N) the inventors tested isolates of these viruses (VIC2089 and VIC4881) for their ability to be neutralised by 3 different convalescent patient plasma samples (DD2, DD3 and DD4), in comparison to the original VIC01 strain (FIG. 6B). DD1 was included as a patient sample that did not contain a measurable neutralising antibody titre. Samples DD2, DD3 and DD4 were impaired in their ability to neutralise the variant strains (VIC2089 and VIC4881). This suggests that patients infected by the original VIC01 strain may have limited immunity against these new variant strains, and furthermore, the emergence of these variants may limit effectiveness and longevity of vaccine-induced immunity as S proteins used in vaccines are based on the Wuhan Index (VIC01) strain.

Next, the inventors tested whether sera from mice immunized with the RBD-Fc dimer vaccine, in the presence of P2C or a-GC adjuvant, was impaired in its ability to neutralize these two variant strains of SARS-COV-2 (FIG. 6C). The results clearly show that these immunized mouse sera samples were capable of neutralizing these variant virus strains at least as well, or better when a-GC was used as an adjuvant, than the original VIC01 strain. To examine a broader range of RBD variants, the inventors used an RBD multiplex assay allowing us to compare binding to 18 different RBD variants that have been detected thus far during the pandemic (FIG. 7). It was found that there was little difference in the binding ability (indicated as the half-maximal effective concentration (EC₅₀)) of sera samples derived from mice immunized with the RBD vaccine combined with either P2C or a-GC adjuvants. The variant with the weakest binding (lowest EC₅₀) was T4781, and even this one showed a high EC₅₀ reading of over 300.

Taken together, vaccination with the RBD-Fc dimer plus adjuvant achieves comparable levels of neutralisation in micro-neutralisation assays against different virus strains (FIG. 5). This is likely to be because the vaccine focusses all of the immune response on RBD epitopes, providing a comprehensive coverage of this target, such that variations in individual amino acid residues are less able to escape vaccine-induced immunity. This may be less likely to happen if the vaccine was whole spike or whole virus based, because there are many more epitopes for the immune system to cover.

### Example 4—Testing of Vaccine in SARS-COV-2 Mouse Challenge Model

### Materials and Methods

### Establishing a SARS-COV-2 Mouse Challenge Model Using VIC2089 (N501Y/D614G)

Groups of 4 BALB/c and C57BL/6 mice were aerosol challenged with either the Index SARS-COV-2 strain VIC01 or the VIC2089 (N501Y/D614G) variant. Three days after challenge mice were killed, right lung lobe harvested, homogenised and supernatants collected and stored at −80° C. Titre of infectious virus (TCID₅₀; 50% tissue culture infectious dose) was determined by titrating the supernatants on Vero cell monolayers that had been seeded in flat-bottom 96-well plates at a concentration of 2×10⁴ cells/well the day before. Serial 10-fold dilutions of SARS-COV-2 were assessed in quadruplicate wells of Vero cells; viral cytopathic effect (CPE) was read 5 days later.

### Assessing Protective Efficacy in the Upper Airways and Lungs of Vaccinated Mice

At specified times post vaccination mice were challenged with VIC2089 (N501Y/D614G) variant via aerosol. At day 3 post-challenge, mice were killed, and blood was collected by cardiac puncture. Left lungs were collected and fixed in 4% paraformaldehyde for histology. To determine viral titres in the lungs and nasal turbinates of challenged mice, the right lung lobe and nasal turbinates were harvested, homogenized by bead-beating in 500 μl media and centrifuged at 2000×g for 5 min. Supernatants were then collected and stored at −80° C. until they were tested. Titre of infectious virus (TCID₅₀; 50% tissue culture infectious dose) was determined by titrating the supernatants on Vero cell monolayers that had been seeded in flat-bottom 96-well plates at a concentration of 2×10⁴ cells/well the day before. Serial 10-fold dilutions of SARS-COV-2 were assessed in quadruplicate wells of Vero cells; viral cytopathic effect (CPE) was read 5 days later.

### Results and Discussion

The VIC2089 strain of SARS-COV-2 carries two important mutations compared to the original VIC01 strain; D614G and N501Y. The latter variation is particularly significant because this amino acid substitution has previously been observed in mouse-adapted SARS-COV-2 viruses. This is because the N501Y variant RBD is capable of binding to mouse ACE2, in contrast to the original ancestral SARS-COV-2 virus with the 501N residue. The inventors therefore tested an isolate of the naturally occurring variant in two commonly used mouse strains (C57BL/6 and BALB/c) (FIG. 8). The results showed that the N501Y variant infected these mice with a high titre of virus peaking around 3 days whereas the original wild-type strain (N501) was unable to infect these mice. Thus, the inventors have developed a small animal model for challenging immunised mice with virus.

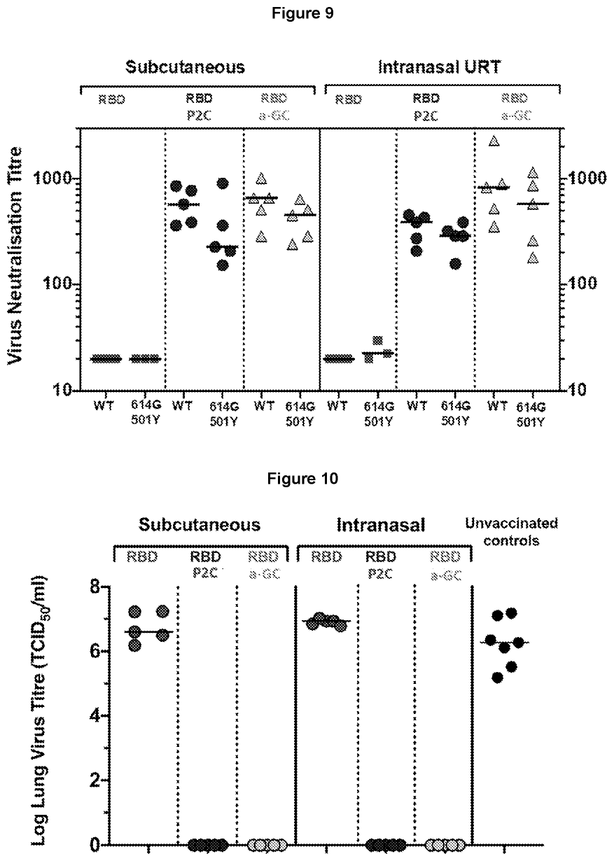

To confirm that serum samples from the immunised mice were capable of neutralising the VIC2089 strain, FIG. 9 shows neutralising titres comparing WT and the VIC2089 strain side by side, with no significant difference observed.

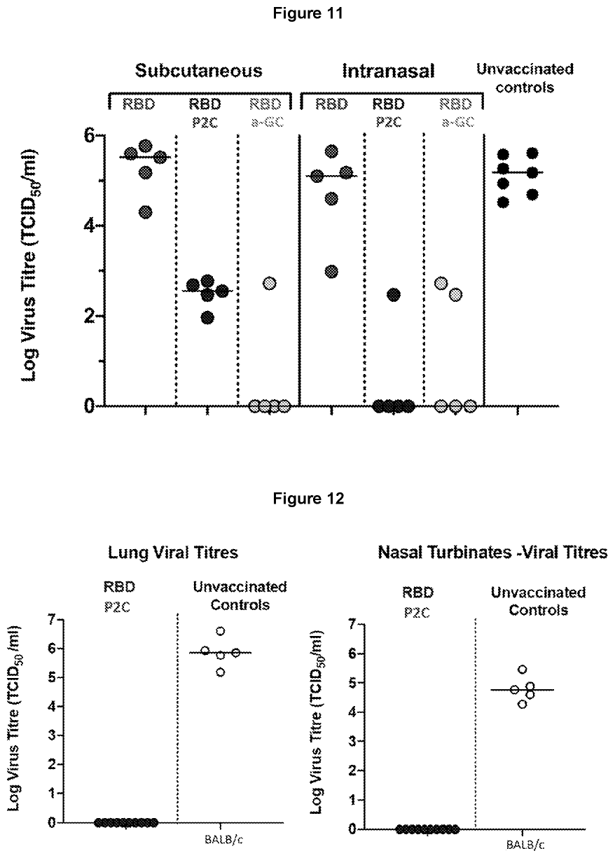

The inventors tested mice that had been previously (approximately 100 days earlier) immunised with the RBD-mouse IgG1-Fc vaccine alone, or with PEG-R4-Pam-2-Cys or α-GalCer adjuvants. Despite the long period between the second immunisation and virus challenge, the mice immunised with RBD plus either adjuvant were completely protected from lung infection, with no detectable virus in lung samples. This was seen regardless of whether the vaccine had been administered subcutaneously or intranasally. RBD vaccine in the absence of the adjuvants provided no protection (FIG. 10). The inventors also tested samples from the mouse nasal turbinates as a measure of upper airway protection. While low levels of virus were still detectable in some of these immunised mice, the virus titres were reduced by at least 99% and in 4/5 mice immunised with RBD plus α-GalCer via the subcutaneous route, there was no detectable virus (FIG. 11).

### Example 5—Assessing Efficacy of Intramuscular Administration of RBD Vaccine

### Materials and Methods

### Vaccination and Challenge Regime

A group of 10 BALB/c mice were vaccinated intramuscularly in 50 μl with 0.1 nmoles of RBD dimer administered with 0.3 nmoles PEG-R4-Pam-2-Cys. Mice were primed on day 0 and boosted on day 28. Four weeks later mice were aerosol challenged with VIC2089 (D614G/N501Y) variant. Five age matched unvaccinated control BALB/c mice were also challenged at the same time.

A group of 5 C57BL/6 mice were vaccinated intramuscularly with 0.1 nmoles of RBD dimer administered with 0.3 nmoles PEG-R4-Pam-2-Cys. Mice were primed on day 0, boosted on day 14 and challenged three months later with VIC2089 (D614G/N501Y) variant. Five age matched unvaccinated control C57BL/6 mice were also challenged at the same time.

### Assessing Protective Efficacy in the Upper Airways and Lungs of Vaccinated Mice

Three days after challenge of both the BALB/c and C57BL/6 mice, animals were killed, the right lung lobe and nasal turbinates were harvested, homogenised and supernatants collected and stored at −80° C. Titre of infectious virus in the tissues (TCID₅₀) was determined by titrating the supernatants on Vero cell monolayers and assessing CPE 5 days later.

### Results and Discussion

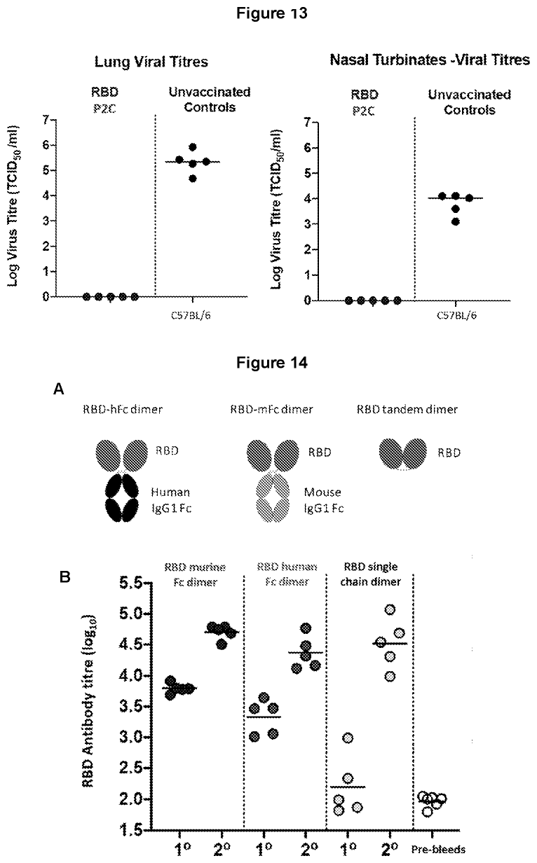

As the injection route in a clinical setting is usually intramuscular, the inventors also tested mice that had been immunised via the intramuscular route (quadracept) in the mouse virus challenge essay. These data impressively showed undetectable virus in both lung and nasal turbinates from 10/10 mice immunised with RBD vaccine plus PEG-R4-Pam-2-Cys (FIG. 13). Similar results were observed for 5/5 of C57BL/6 mice indicating that the vaccine was fully protective across two different laboratory mouse strains.

### Example 6—Comparison of RBD-Murine Fc Dimer, RBD-Human Fc Dimer and Single Chain RBD Dimer

### Materials and Methods

### Vaccination and Bleeding Regime

C57BL/6 mice (5 per group) were vaccinated intramuscularly in 50 μl with 0.1 nmoles of either RBD-mouse Fc dimer, RBD-human Fc dimer or single chain dimer all administered with 0.3 nmoles PEG-R4-Pam-2-Cys. Mice were primed on day 0 and boosted on day 28. Mice were bled just prior to the first injection (pre-bleed), just prior to the second injection (1°), and two weeks following the second injection (2°, day 42). To assess the durability of the antibody response induced by the three different RBD antigens administered with PEG-R4-Pam-2-Cys mice were also bled on days 63 and 82. All serum samples were tested for RBD-specific antibodies by ELISA.

### Results and Discussion

Next, the inventors tested whether there were any differences in the potency of the RBD vaccine based on whether the mouse or human Fc region was used to generate the dimer, and whether a simple single chain dimer would work as well. The inventors compared RBD fused with a mouse IgG1 Fc region, a human IgG1 Fc region, or a single chain RBD dimer (FIG. 14a), in each case with PEG-R4-Pam-2-Cys as the adjuvant. The mouse Fc dimer gave a slightly stronger response than the human Fc dimer after the primary and secondary immunisation whereas the RBD single chain dimer induced a very weak response after the primary immunisation but this rose to similar levels as the Fc dimers after the boost (FIG. 14b). The mouse Fc dimer worked better than human Fc dimer in mice almost certainly because of the higher compatibility of mouse Fc to mouse Fc receptor. However, as humans will be ultimately immunised with the vaccine, it was encouraging to see that the human Fc dimer worked almost as well in mice, and this will likely work better in humans.

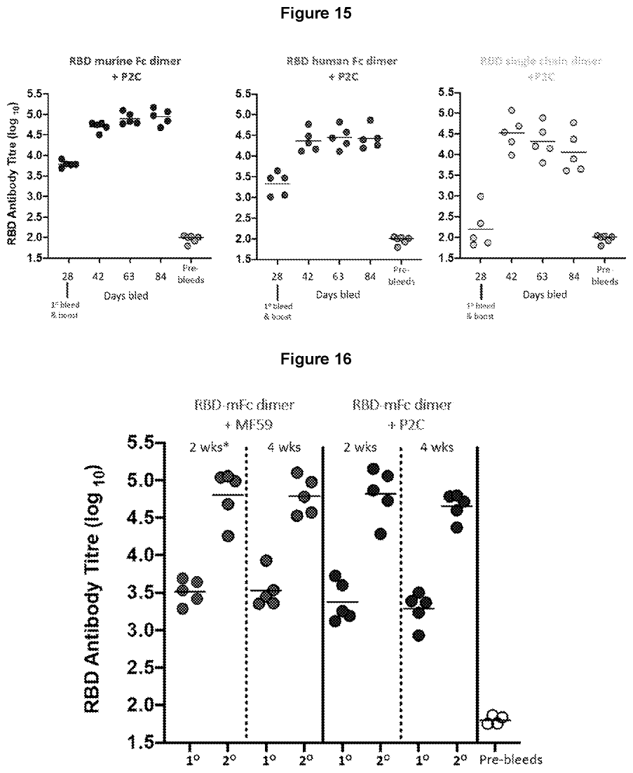

The inventors measured durability of the response to each of the RBD formulations (with PEG-R4-Pam-2-Cys as the adjuvant) (FIG. 15). Both the mouse and human dimers provided stable level of RBD specific antibody titre for up to 84 days from the first injection. In contrast, the antibody levels in mice immunised with the RBD single chain dimer showed a gradual decline with the 84-day time frame, suggesting a less durable response by this dimer. This suggests that, for immunising humans, the RBD-human IgG1 Fc dimer will be the best formulation to provide a strong and durable response.

### Example 7—Testing RBD-Mouse IgG1-Fc Dimer with MF59® Adjuvant

### Materials and Methods

### Vaccination and Bleeding Regime

To assess RBD-specific antibody responses in mice vaccinated with RBD-mouse IgG1-Fc dimer formulated with either MF59® or PEG-R4-Pam-2-Cys, groups of 10 C57BL/6 mice were immunised intramuscularly in 50 μl with 0.1 nmoles RBD mouse Fc dimer and 0.3 nmoles of PEG-R4-Pam-2-Cys, or 0.1 nmoles RBD mouse Fc dimer and MF59® (50% vol/vol). Mice were bled prior to the first immunisation (pre-bleeds), on day 14 (1°) and boosted either on day 14 or day 28, and re-bled two weeks after the secondary immunisation in each case (2°).

To assess RBD-specific antibody responses in mice vaccinated with RBD-mouse IgG1-Fc dimer or RBD single chain dimer formulated with either MF59® or PEG-R4-Pam-2-Cys C57BL/6 mice were immunised intramuscularly with 10 μg RBD mouse Fc dimer or 10 μg of RBD single chain dimer both formulated with either 0.3 nmoles of PEG-R4-Pam-2-Cys or MF59® (50% vol/vol). Mice were primed on day 0 and boosted on day 14. Mice were bled just prior to the first injection (pre-bleed), just prior to the second injection (19), and four weeks following the second injection (2°). All serum samples were tested for RBD-specific antibodies by ELISA.

### Results and Discussion

Because MF59® is a relatively well-established adjuvant the inventors next compared this to the PEG-R4-Pam-2-Cys adjuvant (FIG. 16). Mice were immunised with RBD mouse Fc dimer with either PEG-R4-Pam-2-Cys or MF59® adjuvant. Mice were bled prior to the first immunisation (pre-bleeds) and boosted either two or four weeks later, and re-bled two weeks after the secondary immunisation in each case, and total anti-RBD-specific IgG titres determined. This experiment revealed that MF59® adjuvant was as good as, or better, at inducing high titres of RBD-specific antibody.

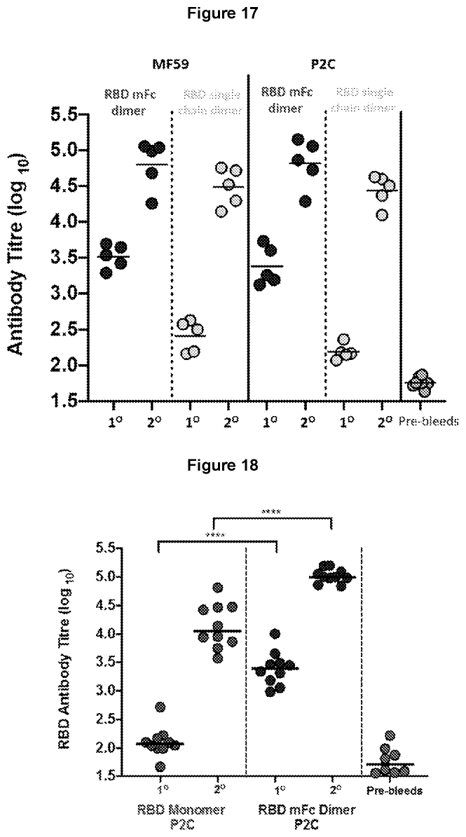

The inventors also compared RBD mouse Fc dimer to RBD single chain dimer in the presence of MF59® adjuvant or PEG-R4-Pam-2-Cys adjuvant (FIG. 17). After both the primary and secondary immunisation, the antibody titres were higher when RBD Fc dimer was used as the vaccine compared to when RBD single chain dimer was used, regardless of which adjuvant was used.

### Example 8—Comprehensive Analysis of Responses to RBD Monomer Versus RBD m-Fc Dimer Administered with PEG-R4-Pam-2-Cys

### Materials and Methods

### Vaccination and Bleeding Regime

Groups of 10 BALB/c mice were immunised intramuscularly in 50 μl with either 0.2 nmoles RBD monomer formulated with 0.3 nmoles PEG-R4-Pam-2-Cys, or 0.1 nmoles RBD mouse Fc dimer formulated with 0.3 nmoles of PEG-R4-Pam-2-Cys. Mice were primed on day 0 and boosted on day 28. Mice were bled just prior to the first injection (pre-bleed), just prior to the second injection (1°), and two weeks following the second injection (2°). Sera were tested for RBD-specific antibodies by ELISA.

### Assessing Protective Efficacy in the Upper Airways and Lungs of Vaccinated Mice

Four weeks after the day 28 boost mice were challenged with VIC2089 (D614G/N501Y) variant. Five age matched unvaccinated control BALB/c mice were also challenged at the same time. Three days after challenge mice were killed, the right lung lobe and nasal turbinates were harvested, homogenised and supernatants collected and stored at −80° C. Titre of infectious virus in the tissues (TCID₅₀) was determined by titrating the supernatants on Vero cell monolayers and assessing CPE 5 days later.

### Results and Discussion

The next experiment was designed to test whether RBD monomer was capable of inducing a similar response to RBD Fc dimer in the presence of adjuvant (FIG. 18). Groups of 10 BALB/c mice were pre-bled, then immunised intramuscularly with either a formulation of vaccine with RBD monomer with PEG-R4-Pam-2-Cys or RBD m-Fc dimer with PEG-R4-Pam-2-Cys. Mice were bled 4 weeks after the primary immunisation, then boosted and bled two weeks later after the secondary immunisation. Serum samples were tested for total anti-RBD antibody titres, which revealed that the RBD Fc dimer yielded a much stronger response after the primary immunisation than did the RBD monomer. The titre of anti-RBD antibody in the serum increased markedly after the boost but still did not get to the same level as the RBD Fc dimer vaccine.

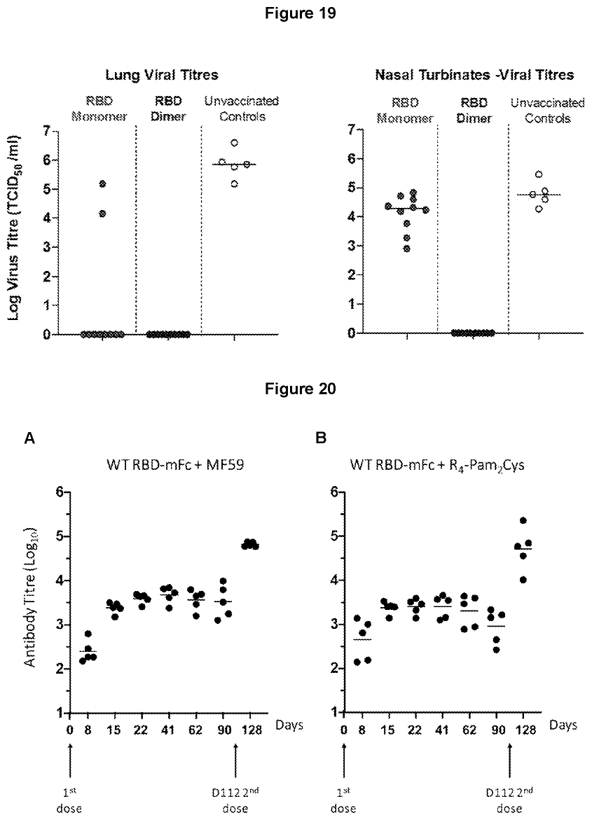

Four weeks after the boost, these mice were tested with the N501Y mouse challenge model (FIG. 19). As before, mice immunised with the RBD dimer were completely protected with no detectable virus in the lung or nasal turbinates. In contrast, while 8/10 mice immunised with RBD monomer showed no detectable virus in their lungs, they all had moderate to high virus titres remaining in their nasal turbinates.

### Example 9—Comparison of the Immunogenicity of the WT RBD-mFc Antigen Formulation with Either the MF59® or PEG-R4-Pam-2-Cys Adjuvant, and Effect of a Dose Regimen and Number

### Materials and Methods

The clinically approved squalene-oil-in-water adjuvant MF59® (Seqirus, Holly Springs, USA) was selected for testing with the WT RBD-mFc antigen due to its long standing and favourable safety record in the Fluad® Quad vaccine.

The WT RBD-mFc dimer vaccine (10 μg) with 0.3 nmoles PEG-R4-Pam-2-Cys adjuvant was compared to WT RBD-mFc dimer vaccine (10 μg) with the 50% vol/vol MF59® adjuvant administered intramuscularly in C57BL/6 mice. Mice were inoculated according to either a two-dose regimen on Day 0 and 112, or a three-dose regimen on Day 0, 22 and 112. Sera was collected pre- and post-inoculation.

### Results and Discussion

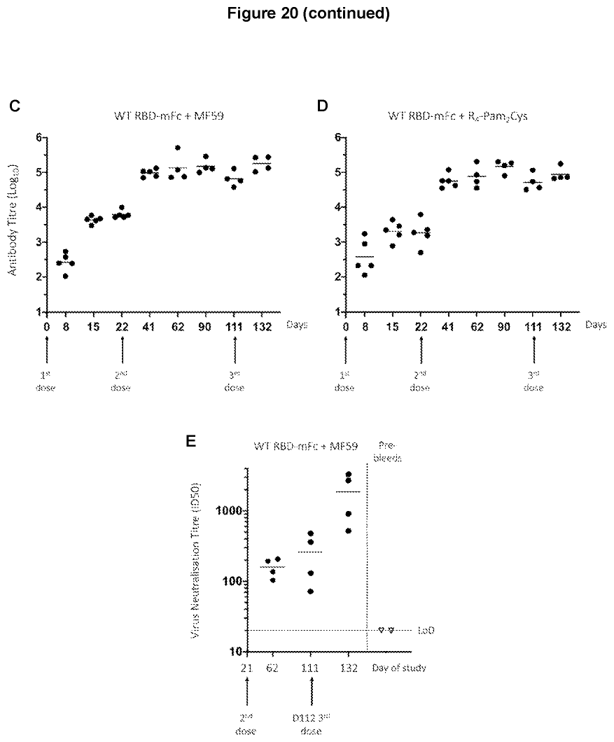

Intramuscular administration of the WT RBD-mFc dimer with either MF59® or PEG-R4-Pam-2-Cys provided comparable total RBD-specific antibody responses. In both groups, enhanced total RBD-specific Ab responses were observed following both the primary and secondary immunisation with the 2-dose schedule (FIG. 20A-B) and the three-dose schedule (FIG. 20C-D).

The extended period between the primary and secondary immunisations with WT RBD-mFc dimer+MF59® in either the two-dose regimen or the three-dose regimen did not appear to impact total RBD-antibody responses following the booster immunisation with similar titres observed 16 days and 12 days after the secondary immunisation in the two-dose (FIG. 20A) and three-dose regime (FIG. 20C), respectively. In the 112 days between the two doses in the two-dose regime, total antibody levels appeared to plateau (FIG. 20A). With the three-dose regimen total antibody responses there was a trend for total RBD-specific Ab responses to increase from secondary immunisation (day 22) to day 90 and then reduce slightly on day 111 (89 days after secondary immunisation). The third dose administered on Day 111 reversed the trend in decline and total RBD-specific Ab responses increased again by day 132 (FIG. 20C).

Results of the virus neutralisation titres from mice inoculated with WT RBD-mFc dimer+MF59® in a three-dose regime demonstrated an increasing level of SARS-CoV-2-specific nAbs from 40 days post-secondary immunisation (Day 62) and increased further 21 days following the tertiary immunisation (Day 132) (FIG. 20E).

These results demonstrate the immunogenicity of the WT RBD-mFc dimer is maintained when used with the clinically approved MF59® adjuvant when compared to the PEG-R4-Pam-2-Cys adjuvant. In addition, antibody levels appear similar when a booster is administered 22 or 112 days following the priming immunisation. A third booster immunisation appears to be able to reverse any decline in total antibody levels and enhance nAb levels further. This reflects the ability of WT RBD-mFc dimer to provide a boost that raises total and nAb levels higher than they were after the second injection.

### Experiment 10—Assessing the Immunogenicity and Protective Efficacy of Increasing Doses of WT RBD-mFc with MF59® after One Dose

### Materials and Method

To determine whether doses greater than 10 μg of WT RBD-mFc, with or without the MF59® adjuvant, could provide improved protective immunity after a single vaccination, C57BL/6 mice were immunised via the intramuscular route using a prime only regimen with either 10, 30 or 50 μg of the WT RBD-mFc in the presence or absence of MF59® (50% v/v).

### Results and Discussion

Results indicated a single dose of either 10 or 30 μg WT RBD-mFc plus MF59® was not sufficient to provide protective immunity where virus titres were detected in the lungs of all challenged animal. In addition, when compared to the 10 μg dose of WT RBD-mFc plus MF59®, the higher 30 μg dose did not provide superior protective immunity against SARS-COV-2 viral challenge with higher virus titres comparable to those observed in unvaccinated controls.

These results demonstrate that increasing the dose of WT RBD-mFc plus MF59® in the priming vaccine does not overcome the need for a second vaccination for induction of a protective immune response in naïve mice.

### Experiment 11—Comparison of the Immunogenicity and Protective Efficacy of RBD-Human IgG1 Fc Dimers to RBD-Mouse IgG1 Fc Dimers

### Materials and Method

To further progress the WT RBD-Fc vaccine candidate to human clinical trials, a WT RBD-human Fc dimer protein (using human IgG1 Fc region) was generated. Intramuscular administration of 10 μg WT RBD-hFc dimer+PEG-R4-Pam-2-Cys was compared with 10 μg WT RBD-mFc dimer+PEG-R4-Pam-2-Cys in both BALB/c and C57BL/6 mice strains using a prime-boost regime.

### Results and Discussion

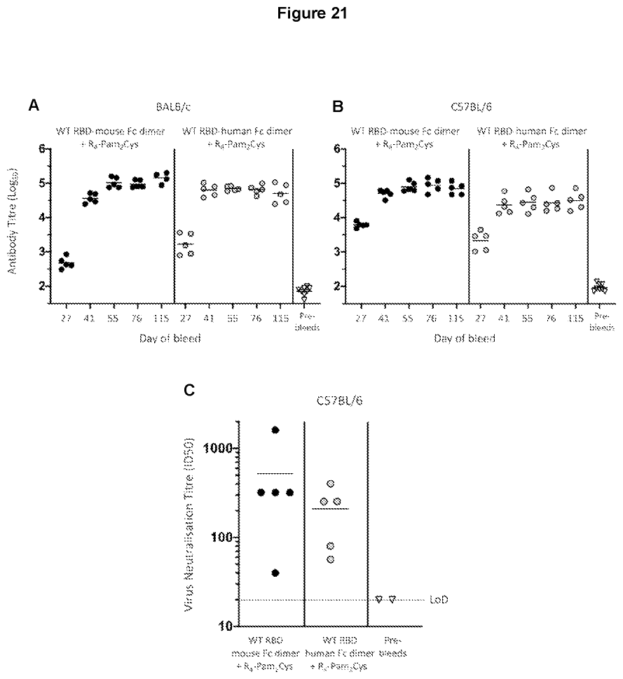

RBD-specific antibody analysis demonstrated that the human Fc form of the RBD-Fc vaccine induced a similar level and pattern of total anti-RBD antibodies to the mouse Fc form, albeit slightly lower in C57BL/6 mice. In both mouse strains the mouse and human Fc form of the RBD vaccine induced durable antibody responses that were generally maintained at a constant level up to day 115 (87 days post-boost) (FIG. 21A-B).

Neutralising Ab (nAb) titres assessed in C57BL/6 mice collected 13 days after the second immunisation demonstrated elevated nAbs in all animals and as observed with total RBD-specific antibodies, the human Fc form of the RBD vaccine induced a similar, or slightly lower level of nAbs to the mouse Fc form of the vaccine (FIG. 21C).

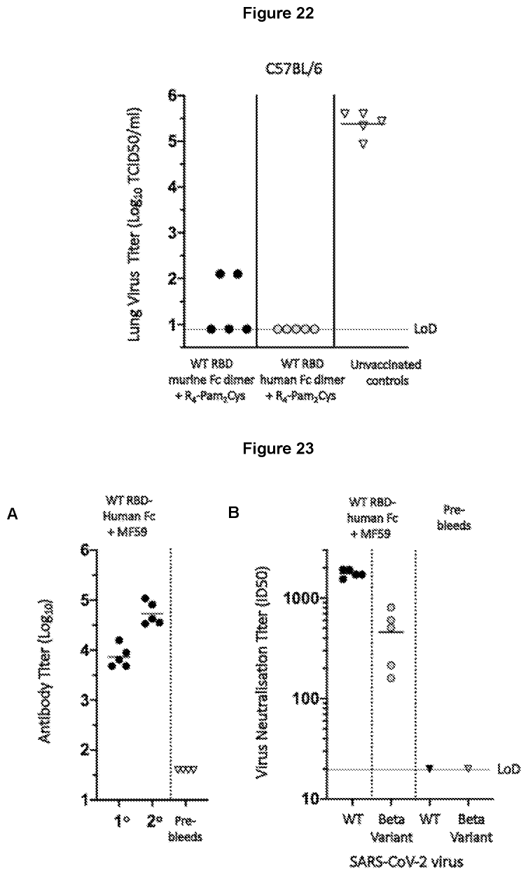

The mouse SARS-COV-2 challenge model was applied on Day 139 (11 days post-booster immunisation) and results demonstrated both the mouse and human forms of the RBD-Fc vaccine in the presence of the +PEG-R4-Pam-2-Cys adjuvant were capable of complete protection against lower airway infection in 5/5 mice immunised with RBD-human Fc and 3/5 mice immunised with RBD-mouse Fc (FIG. 22).

These results demonstrate the WT RBD-human Fc dimer protein provided similar immunogenicity and protective efficacy to the WT RBD-mouse Fc dimer in the presence of PEG-R4-Pam-2-Cys adjuvant.

### Experiment 12—Immunogenicity and Protective Efficacy of WT RBD-hFc with MF59®

The immunogenicity and protective efficacy of the WT RBD-human FC plus the MF59® adjuvant was next assessed in C57BL/6 mice via the intramuscular route using a prime-boost regimen.

The WT RBD-hFc+MF59® vaccine candidate provided high levels of RBD-specific total Abs 20 days post-primary immunisation (day 20) that elevated further 12 days post-boost immunisation (Day 33) (FIG. 23A). High nAb levels capable of neutralising both the WT virus and the Beta variant virus were also observed (FIG. 23B). As expected, higher nAbs against the WT virus were observed compared to the Beta virus.

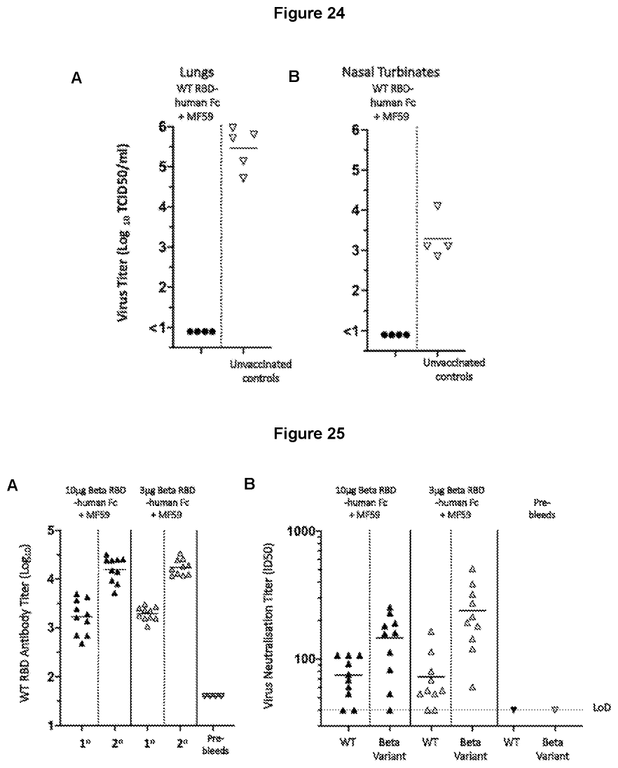

The mouse SARS-COV-2 challenge model was applied on day 81 (60 days post-secondary immunisation) and complete protection against both upper and lower airway infections were indicated (FIG. 24).

These results demonstrate the WT RBD-hFc dimer plus MF59® adjuvant can provide protective immunity to SARS-COV-2 in mice when administered intramuscularly with a prime/boost regimen.

### Experiment 13—Immunogenicity of the Beta RBD-hFc Plus MF59® Vaccine Candidate

### Materials and Methods

Since onset of the pandemic, SARS-COV-2 variants have replaced the original WT strain of the virus. Some of these variants, including the Beta and Delta strains, appear to be partially resistant to vaccine-induced immunity mediated by WT based vaccines. The Beta variant is the most evasive variant described to date and carries three mutations in the RBD: N501Y, E484K and K417N.

To demonstrate the adaptability of the RBD-hFc+MF59® vaccine, a Beta RBD version of the vaccine was generated (Beta RBD-hFc). Two doses (10 μg and 3 μg) of the subsequent Beta RBD-hFc+MF59® vaccine were investigated in mice via the intramuscular route in a prime-boost regimen.

### Results and Discussion

A robust total antibody response against the WT RBD monomer was observed following the prime-boost regimen in mice immunised with either the 10 μg or 3 μg dose of Beta RBD-hFc+MF59® (FIG. 25A).

Neutralising antibody titres specific for the Beta variant strain were observed in 19/20 mice immunised with either the 3 μg or 10 μg of Beta RBD-hFc+MF59® (FIG. 25B). Interestingly, mice prime-boosted with the 3 μg dose appeared to have higher nAbs for the Beta variant compared to mice prime-boosted with the 10 μg dose. In addition, nAbs capable of neutralising the WT variant was observed in 16/20 mice, albeit at lower effective titres (FIG. 25B).

These results indicate that intramuscular administration of the Beta RBD-hFc dimer plus MF59® in mice using a prime-boost regimen produces a robust total WT-RBD specific antibody response and nAbs capable of neutralising both WT and Beta variants of SARS-COV-2.

### Experiment 14—Assessing the Immunogenicity and Protective Efficacy of Different Doses of WT RBD-hFc Antigen and Beta Variant RBD-hFc Antigen Administered with MF59®

### Materials and Methods

To determine the minimal effective dose of the WT RBD-hFc dimer and the Beta RBD-hFc dimer, C57BL/6 mice were immunised intramuscularly with either dimer at a range of doses (10, 3, 1 and 0.3 μg) in the presence of MF59® using a prime-boost regimen.

### Results and Discussion

### Immunogenicity

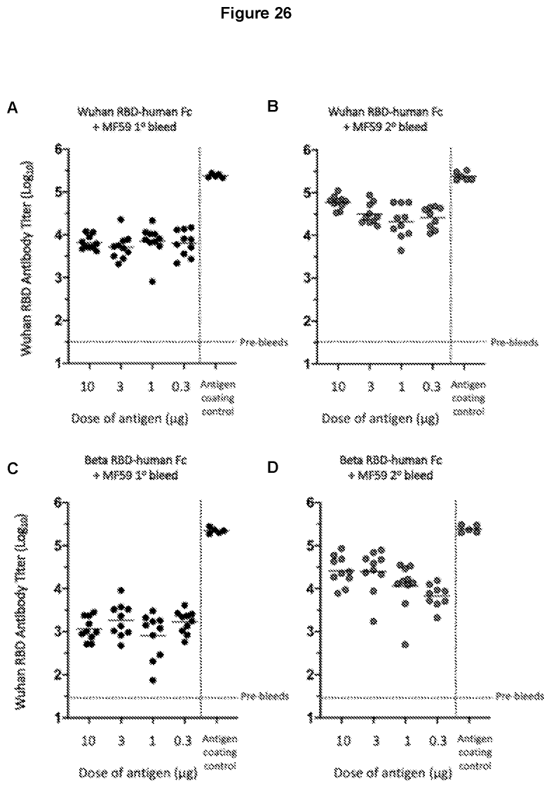

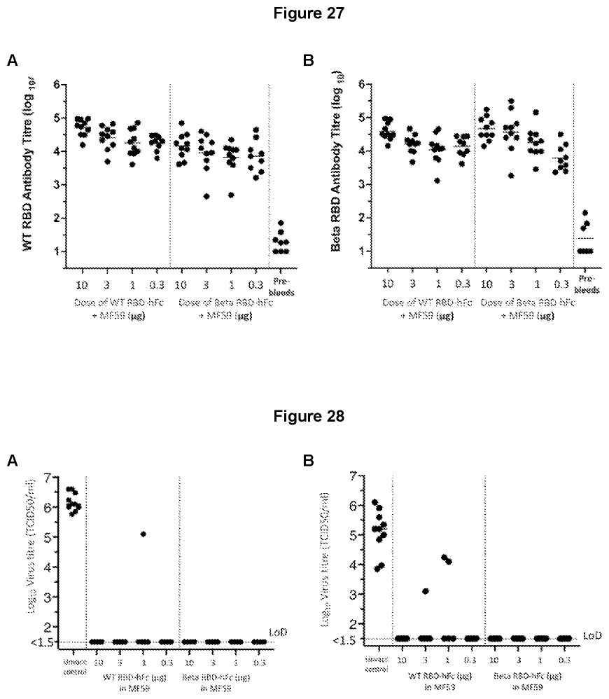

Total anti-WT RBD antibody titres in primary and secondary sera of mice vaccinated intramuscularly with 10, 3, 1 or 0.3 μg of either WT RBD-hFc or Beta RBD-hFc in the presence of MF59® were determined using an RBD-specific ELISA (FIG. 14). Primary RBD antibody responses were relatively consistent across all the doses of WT RBD-hFc) (FIG. 26) and Beta RBD-hFc (FIG. 26) assessed, although the overall titres were slightly lower in the mice immunised with the Beta RBD-hFc formulations. Secondary RBD antibody titres were boosted across all groups with a slight trend towards a decrease in the level of boosting as the dose of antigen decreased (FIG. 26C,D). Similarly, after boosting the overall RBD antibody titres were slightly lower in the mice immunised with the Beta RBD-hFc formulations and there was also a greater degree of variability observed within each of the Beta RBD-hFc vaccinated groups. Similar results were observed when secondary (day 56) sera from WT RBD-hFc+MF59® or Beta RBD-hFc+MF59® vaccinated mice were analysed for Ab responses against both the WT RBD monomer and the Beta RBD monomer (FIG. 27).

### Protective Efficacy

Protective efficacy against lower airways infection was assessed in a mouse SARS-COV-2 challenge model using VIC2089 (N501Y/D614G) and the Beta variant B.1.351 as challenge strains. On day 76 of the study (55 days after the boost) 5 of the 10 mice from each vaccination group immunised with 10, 3, 1 or 0.3 μg of either WT RBD-hFc or Beta RBD-hFc in the presence of MF59® were aerosol challenged with VIC2089 (FIG. 28A). On day 83 of the study (62 days after the boost) the remaining 5 mice in each group were aerosol challenged with B.1.351 (FIG. 28B).

### Experiment 15—Assessing the Immunogenicity and Protective Efficacy of RBD-hFc Dimer Vaccines in a Hamster Model

### Materials and Methods

Hamsters infected with SARS-COV-2 closely resemble the manifestations of upper and lower respiratory tract infection in humans. They also lose weight, become lethargic, and develop ruffled fur, a hunched posture, and rapid breathing. High levels of SARS-COV-2 have been found in the hamsters' lungs and intestines, which are tissues studded with ACE2 that is capable of binding to the SARS-COV-2 virus. For this reason, hamsters have been used as a model to evaluate COVID vaccines during development.

The hamsters were initially immunised on day 0 and 21 with 30 or 10 μg of WT RBD-hFc dimer formulated with either MF59® or PEG-R4-Pam-2-Cys. SARS-COV-2 challenge was conducted in accordance with the methods of Zhou et al., Cell Host & Microbe, 2021, 29:551-563 per “Syrian Hamster Experiments”.

### Results and Discussion

### Total RBD-Specific Antibody Responses

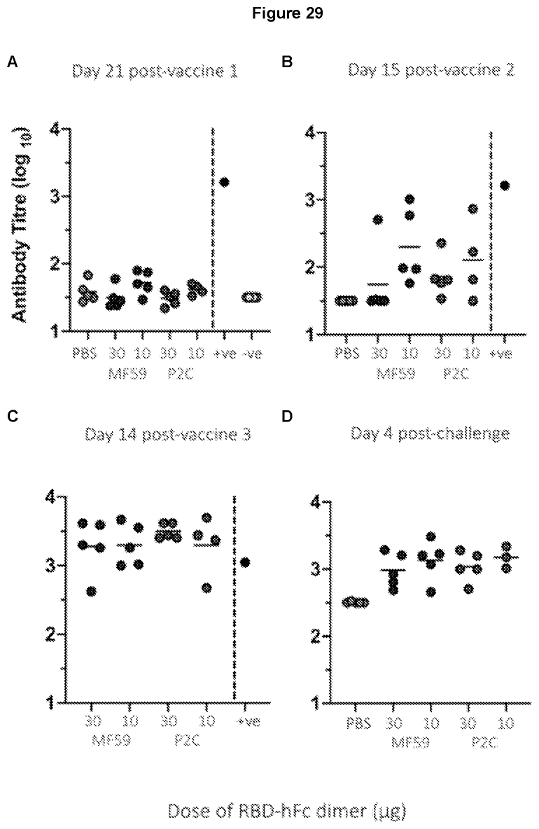

All hamsters sero-converted after the second injection, although the responses for several hamsters were lower and with greater variability than observed in previous mouse studies. Therefore, the hamsters were administered a third dose of the corresponding vaccine, on day 49. This resulted in much higher antibody production in all hamsters presented in FIG. 29. Interestingly, following the second vaccine dose, the 30 μg WT RBD-hFc dimer+MF59® group had lower antibody titres than did the 10 μg WT RBD-hFc dimer+MF59® group. The inverse response was also observed in the mouse studies.

### Experiment 16—Assessing the Immunogenicity and Protective Efficacy of RBD Vaccines Against the Beta Variant in a Hamster Model

### Materials and Methods

Hamsters were vaccinated with 30, 10 or 3 μg of the WT RBD-hFc dimer+MF59® or Beta RBD-hFc dimer+MF59®, receiving 2 intramuscular doses on days 0 and 21. A 3^{rd} dose was administered on day 63, and challenge performed on day 85 with tissues harvested on day 88 for determination of viral load in the lungs and nasal turbinates of each hamster. Neutralising antibody (nAb) titres were assessed as described elsewhere in the specification.

### Results and Discussion

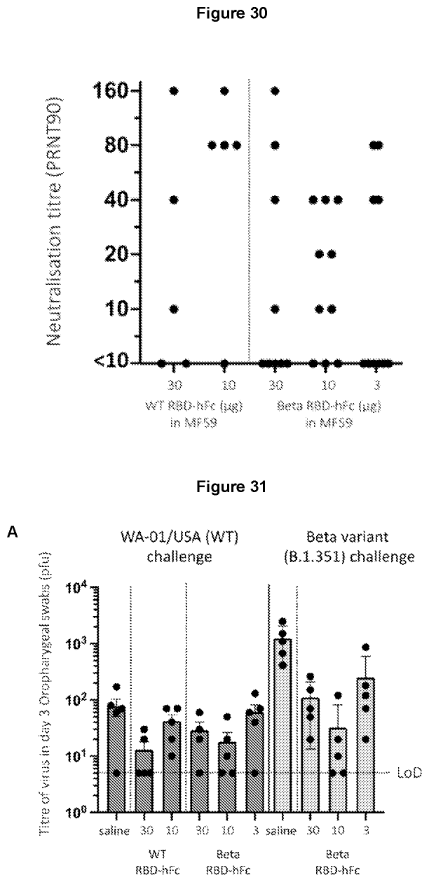

FIG. 30 displays the individual nAb titres of the hamsters in each group. At this timepoint, 7 of the 10 hamsters immunised with WT RBD-hFc dimer+MF59® had detectable nAb responses and 15 of the 30 mice immunised with WT RBD-hFc dimer+MF59® had detectable nAb responses. These nAb responses were measured against WT virus.

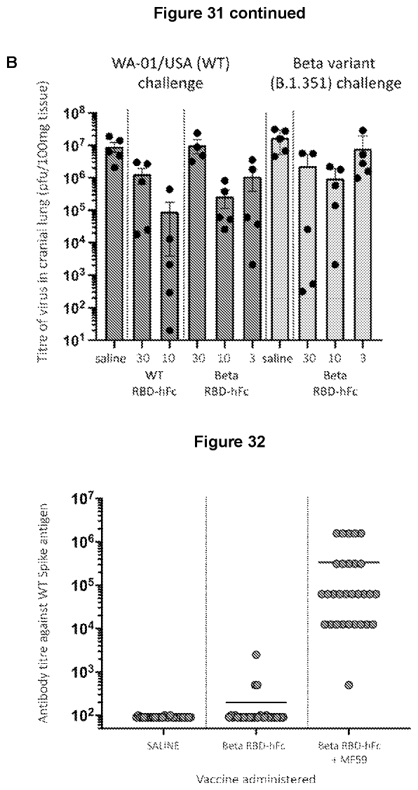

Next, these hamsters were then challenged with either WT (WA-01/USA) or Beta variant (B.1.351) SARS-COV-2 virus on day 85 and oropharyngeal swabs collected every day for 3 days post challenge. Hamsters were sacrificed on day 88 for lung virus titre assessment. Data is presented for day 3 swabs data and viral load/mg lung tissue at end of experiment. Consistent with the moderate and highly varied nAb response observed (FIG. 30), the immunised hamsters also showed a moderate and variable degree of resistance to viral challenge (FIG. 31). For the WT SARS-COV-2 virus challenge, the viral load in the hamsters administered PBS-(placebo) in the day 3 swabs was low (mean ˜80 PFU) and one control hamster was below the limit of detection of 5 PFU, which left little scope to show a clear reduction with the vaccinated hamsters. Nonetheless, the mean viral load in day 3 swabs was lower (13 PFU) in the hamsters immunised with 30 μg WT RBD-hFc+MF59® where 3 hamsters had PFU below the limit of detection (5 PFU) and 2 had low but detectable virus. Hamsters treated with 10 μg WT RBD-hFc+MF59® all had detectable virus and the mean viral load in day 3 swabs (42 PFU) was only moderately lower than that of the PBS-treated hamsters. Hamsters immunised with 30, 10 and 3 μg Beta SARS-COV-2 RBD-hFc+MF59® showed only moderate reductions in mean PFU (29, 18, 61 PFU) (FIG. 31A).

Hamsters challenged with the Beta (B.1.351) SARS-COV-2 virus had higher viral loads in the PBS-treated group (mean 1200 PFU) and showed better evidence of vaccine-induced protection in the day 3 swab samples. All hamsters immunised with Beta RBD-hFc+MF59®, except for one animal that received the lowest 3 μg dose, had lower viral loads with no overlap with the PBS group (mean PFU of 110, 32 and 250 from the 30, 10 and 3 μg treated groups, respectively) (FIG. 31A).

Results were obtained from the sample of cranial lung tissue were similar, but with some notable differences to the 3-day swab samples (FIG. 31B). Thus, the 10 μg WT RBD-hFc+MF59® group showed the best protection against WT (WA-01/USA) SARS-COV-2 challenge (9.1×10\hat{ }4 versus 9.2×10\hat{ }6 PFU/100 mg tissue in the PBS-treated group) and the 10 μg and 3 μg Beta RBD-hFc+MF59® groups had lower mean titres (2.7×10\hat{ }5 and 1.1×10\hat{ }6) than the 30 μg Beta RBD+MF59® group (1×10\hat{ }7 PFU/100 mg tissue). For the Beta virus challenge groups, the PBS treated group had mean 1.7×10\hat{ }7 PFU/100 mg tissue), whereas the lowest mean PFU came from the 10 μg Beta RBD-hFc+MF59® immunised group (9.4×10\hat{ }5). The 30 μg Beta RBD-hFc+MF59® immunised group exhibited a large spread where three hamsters were much lower (100-fold or more) than the PBS group whereas two were similar to the PBS group, giving a mean PFU value of 2.3×10⁶. Little to no evidence of protection was seen in hamsters in the lowest 3 μg Beta RBD-hFc+MF59® immunised group (7.9×10⁶ PFU).

Taken together, the RBD-hFc+MF59® vaccine was shown to be immunogenic in the hamster studies (FIGS. 29-30).

### Experiment 17—Immunogenicity of the Beta RBD Vaccine in a Rat Model

### Materials and Methods

As part of a toxicology study, the immunogenicity of the Beta RBD-hFc vaccine with or without MF59® adjuvant in a rat model was assessed (FIG. 32). Three groups of 30 Sprague Dawley Rats were vaccinated intramuscularly on days 0, 22 and 43 with saline, 50 μg of Beta RBD-hFc or 50 μg of Beta RBD-hFc+MF59® adjuvant. At termination of the toxicity on days 44-45, or day 56, a terminal blood sample was collected from each rat and processed to serum and assessed for WT SARS-COV-2 spike-specific IgG antibody responses by ELISA.

### Results and Discussion

As shown in FIG. 32, the Beta RBD-hFc vaccine administered with MF59® was highly immunogenic, and all but one of the 30 rats immunised with Beta RBD-hFc vaccine+MF59® produced titres of spike-reactive IgG antibodies at 10⁴ or higher. One rat produced lower titre of antibody, but this was still clearly higher than the background levels (titre of 100) in the saline injected control animals. In contrast, only 3 of 30 rats that were immunised with Beta RBD hFc without MF59® adjuvant produced detectable IgG anti-spike antibody.

### Experiment 18—Assessing the Stability of Lyophilised RBD-Human Fc/PEG-R4-Pam-2-Cys Vaccine Formulations

### Methods and Materials

The inventors assessed the stability of freeze-dried RBD-human Fc dimers formulated with PEG-R4-Pam-2-Cys. These formulations were stored at 40° C., room temperature (RT), 4° C. or −20° C. for 1 month prior to being reconstituted and used for intranasal vaccination of BALB/c mice. Responses to stored freeze-dried formulations were compared to responses elicited by freshly prepared RBD-human Fc dimers formulated with PEG-R4-Pam-2-Cys, and freeze-dried formulations reconstituted 24 hours after lyophilisation.

Groups of 5 BALB/c mice were vaccinated intranasally with 2 doses of either freshly prepared vaccines, freeze-dried vaccines reconstituted 24 hours after lyophilisation or reconstituted freeze-dried vaccines stored at 40° C., RT, 4° C. or −20° C. for 4 weeks. The vaccines were administered on days 0 and 30. The lyophilised, stored vaccines for administration of the first dose were prepared on day-32 and lyophilised, stored vaccines for administration of the second dose were prepared on day 0 of the experiment. This allowed vaccines for both the first and second dose to be stored at the specified conditions for 1 month. Mice were bled just prior to the first injection (pre-bleed), 21 days after the first immunisation (1° bleeds), and at various timepoints following the second immunisation (2° bleeds).

Humoral responses were evaluated after the first and second immunisation and included anti-RBD antibody responses assessed by ELISA, as well as evaluation of SARS-COV-2 neutralising antibodies measured by an in vitro microneutralisation assay and by a surrogate virus neutralising test (GenScript, USA).

On day 112 of the study (82 days after the second dose) mice inoculated with freshly prepared RBD-hFc/PEG-R4-Pam-2-Cys formulations and mice vaccinated with RBD-hFc/PEG-R4-Pam-2-Cys formulations lyophilised and stored at 40° C. for 1 month, were challenged with VIC2089 (D614G/N501Y).

### Results and Discussion

### Total Immunoglobulin (Lg) Reactive to WT RBD Monomer, and Neutralising Ab Responses

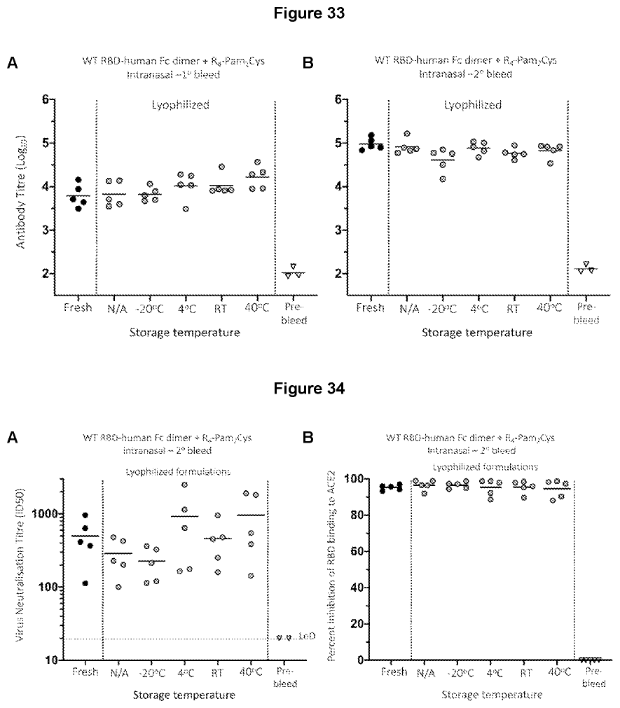

Primary and secondary WT RBD-specific antibody responses in the sera of mice vaccinated with either freshly prepared PEG-R4-Pam-2-Cys/RBD-hFc or freeze-dried PEG-R4-Pam-2-Cys/RBD-hFc formulations were assessed. All mice receiving lyophilized formulations stored under different conditions exhibited strong antibody titres that were comparable to the titres generated in mice vaccinated with the freshly prepared formulation (FIG. 33).

Similarly, when the neutralising antibody responses in the sera of mice receiving freshly prepared PEG-R4-Pam-2-Cys+RBD-hFc were compared to neutralising responses in animals vaccinated with the freeze-dried PEG-R4-Pam-2-Cys+RBD-hFc formulations, comparable neutralising responses were observed amongst all the mice using both a microneutralisation assay (FIG. 34A) and a surrogate virus neutralisation test (FIG. 34B).

### Protective Efficacy

Protective efficacy against upper and lower airways infection was assessed in the mouse SARS-COV-2 challenge model using VIC2089 (N501Y/D614G). Mice vaccinated intranasally on days 0 and 30 with WT RBD-hFc+PEG-R4-Pam-2-Cys or lyophilized WT RBD-hFc+PEG-R4-Pam-2-Cys stored at 40° C. for one month, were aerosol challenged with VIC2089, 82 days after the second immunisation. Age and sex matched unvaccinated control mice were also challenged at the same time. Three days after challenge mice were killed, and the titre of infectious virus (TCID50) in the lungs and nasal turbinates of individual mice were determined by titrating lung and nasal supernatants on Vero cell monolayers and measuring viral CPE 5 days later.

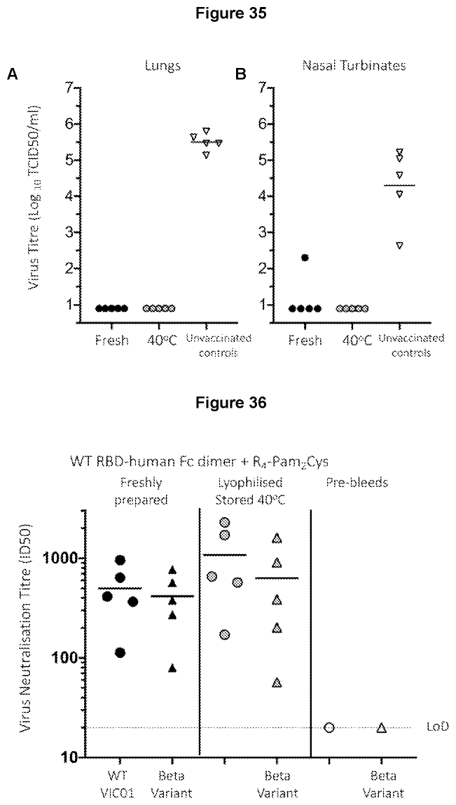

Complete protection against lung infection was observed in all BALB/c mice inoculated with either freshly prepared or lyophilised PEG-R4-Pam-2-Cys/RBD-mFc vaccine stored at 40° C. (FIG. 35A). Furthermore, no virus was detected in nasal turbinates of 4/5 mice receiving the freshly prepared vaccine and 5/5 mice immunised with the lyophilised vaccine (FIG. 35B).

From these results the inventors conclude that immunogenicity and protective efficacy are maintained when PEG-R4-Pam-2-Cys+RBD-mFc formulations are lyophilized and stored for up to 1 month at temperatures ranging from −20° C. to 40° C.

### Neutralising Activity Against Wuhan Index Strain VIC01 and Beta Variant B1.351

Secondary (day 70) sera from mice vaccinated intranasally with freshly prepared WT RBD-hFc+PEG-R4-Pam-2-Cys or lyophilized WT RBD-hFc+PEG-R4-Pam-2-Cys stored at 40° C. were assessed for their ability to neutralise both the Wuhan Index strain VIC01 and Beta variant B.1.351 via microneutralisation assay. As shown in FIG. 36 both formulations despite containing the Wuhan Index strain RBD antigen elicited potent neutralising activity against both the homologous Wuhan Index strain VIC01 and the beta variant B.1.351.

1-49. (canceled)

50. A chimeric or fusion protein for inducing an immune response to coronavirus,
wherein the chimeric or fusion protein comprises a dimer of receptor binding domains from a Spike protein of a coronavirus linked to an Fc region of an antibody, or
wherein the chimeric or fusion protein comprises a dimer of receptor binding domains from a Spike protein of a coronavirus linked to a polypeptide comprising an Fc receptor binding domain.

51. A chimeric or fusion protein according to claim 50, wherein the receptor binding domain comprises an amino acid sequence from a WT, alpha, beta, gamma, kappa or delta SARS-COV-2 strain or variant or any other strain or variant defined in Table 3.

52. A chimeric or fusion protein according to claim 50, wherein the receptor binding domain comprises an amino acid sequence from a WT SARS-COV-2 strain.

53. A chimeric or fusion protein according to claim 50, wherein the receptor binding domain comprises an amino acid sequence from a beta SARS-COV-2 strain.

54. (canceled)

55. (canceled)

56. A chimeric or fusion protein according to claim 50, wherein the receptor binding domain from a Spike protein of a coronavirus comprises, consists essentially of or consists of an amino acid sequence having at least 80% identity to any one of SEQ ID NO: 15 or 28.

57. (canceled)

58. (canceled)

59. A chimeric or fusion protein according to claim 50, wherein the Fc region of the antibody is an Fc region of an IgG.

60. A chimeric or fusion protein according to claim 59, wherein the IgG is IgG1.

61. (canceled)

62. A chimeric or fusion protein according to claim 50, wherein the Fc region of the chimeric or fusion protein comprises two heavy chain fragments, more preferably the CH2 and CH3 domains of said heavy chain.

63. A chimeric or fusion protein according to claim 50, wherein the Fc region of an antibody comprises, consists essentially of or consists of an amino acid sequence having at least 80% identity to any one of SEQ ID NO: 16 or 18.

64. (canceled)

65. A chimeric or fusion protein according to claim 50, wherein the chimeric or fusion protein comprises, consists essentially of or consists of a sequence as set forth in any one of SEQ ID NO: 3, 22 or 23.

66. A chimeric or fusion protein according to claim 50, wherein the receptor binding domain from the Spike protein of a coronavirus consists of SEQ ID NO: 15 or 28.

67. A composition comprising: a chimeric or fusion protein according to claim 50, and an adjuvant.

68. A composition according to claim 67, wherein the adjuvant is a TLR2-agonist.

69. A composition according to claim 68, wherein the TLR2-agonist is a Pam-2-Cys containing molecule such as PEG-R4-Pam-2-Cys, or a stimulator of NKT cells.

70. A composition according to claim 67, wherein the adjuvant is selected from the group consisting of Pam3CSK4, PEG-R4-Pam-2-Cys, MALP-2, lipoteichoic acid, OspA, Porin, LcrV, lipomannan, Lysophosphatidylserine, Lipophosphoglycan (LPG), Glycophosphatidylinositol (GPI) and Zymosan.

71. A composition according to claim 67, wherein the adjuvant is selected from the group consisting of poly-I: C, CpG, poly-ICLC, 1018 ISS, aluminum salts, Amplivax, AS15, B (C, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59®, AddaVax™, monophosphoryl lipid A, Montanide IMS 1312, Montanide ISA 206, Montanide ISA V, Montanide ISA-51, OK-432, OM-174, OM-197-MP-EC, ONTAK. PEPTEL, vector system, PLGA microparticles, resiquimod, SRL172, Virosomes and other Virus-like particles, YF-17D, VEGF trap, R848, beta-glucan, Pam-3-Cys, and Aquila's QS21 stimulon.

72. A composition according to claim 71, wherein the adjuvant is MF59®.

73. A method for inducing an immune response to a coronavirus in a subject, the method comprising administering to the subject a chimeric or fusion protein according to claim 50, thereby inducing an immune response in the subject to the coronavirus.

74. A method according to claim 73, wherein the coronavirus infection is a SARS-COV-2 infection.

75. A method according to claim 73, wherein the chimeric or fusion protein is administered in combination with an adjuvant.


SEQUENCE LISTING (33 sequences)

SEQ ID NO: 1 - DNA, 1383 bp, Artificial Sequence
  RBD-mouse IgG1 Fc-fusion nucleotide sequence
gaattcaagcttgccaccatggggattcttcccagccctgggatgcctgcgctgctctccctcgtgagccttctctccgtgctgctgatgggttgcgtagctgaaaccggtaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaactctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctggatccgggagcggctgcaaaccctgtatctgcaccgtgcctgaagtctcctccgtctttatcttcccccccaagcctaaggacgtcctgaccatcaccctcacccctaaggtcacctgcgtcgtggtcgatatatccaaagatgaccccgaggtgcagttcagctggttcgtggacgatgtggaagtccacaccgcccagacccaacccagggaggagcagttcaactccaccttcaggtccgtgagcgagctgcccattatgcaccaggactggctcaacggaaaggaatttaagtgtagagtcaacagcgctgccttccccgcccccatcgaaaagacaatctccaagaccaagggcaggcctaaagctcctcaggtctacaccatccccccccccaaggagcagatggccaaggacaaggtgagcctcacctgcatgatcacagacttcttccccgaggatataaccgtggagtggcagtggaatggccagcccgccgagaactataagaacacccagcccattatggacaccgacggcagctatttcgtctatagcaagctgaacgtccagaagtccaattgggaagccggcaacacctttacctgctccgtgctgcatgagggcctgcacaatcaccacaccgagaagagcctgagccactcccccggcaagtgatgactcgag

SEQ ID NO: 2 - protein, 459 aa, Artificial Sequence
  RBD-mouse IgG1 Fc-fusion amino acid sequence
EFKLATMGILPSPGMPALLSLVSLLSVLLMGCVAETGNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSGSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKLE

SEQ ID NO: 3 - protein, 423 aa, Artificial Sequence
  Purified RBD-mouse IgG1 Fc-fusion monomer amino acid sequence
ETGNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSGSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 4 - DNA, 1395 bp, Artificial Sequence
  RBD-human IgG1 Fc-fusion nucleotide sequence
gaattcaagcttgccaccatggggatacttcccagccctgggatgcctgcgctgctctccctcgtgagccttctctccgtgctgctgatgggttgcgtagctgaagctagcaatctgtgccccttcggcgaggtgttcaacgccacaagattcgcctctgtgtacgcctggaaccggaagcggatcagcaattgcgtggccgactacagcgtgctgtacaacagcgccagcttcagcaccttcaagtgctacggcgtgtcccctaccaagctgaacgacctgtgcttcaccaacgtgtacgccgacagcttcgtgatcagaggcgacgaagtgcggcagattgcccctggacagacaggcaagatcgccgattacaactacaagctgcccgacgacttcaccggctgtgtgattgcctggaacagcaacaacctggacagcaaagtcggcggcaactacaactacctgtaccggctgttccgcaagagcaacctgaagcctttcgagcgggacatcagcaccgaaatctaccaggccggcagcaccccttgcaatggcgtggaaggcttcaactgctacttcccactgcaaagctacggcttccagcctacaaacggcgtgggctaccagccttacagagtggtggtgctgagcttcgagctgctgcatgctcctgccacagtgtgtggacctggatccggcagcggtacccacacctgtcctccatgtcctgctccagaactgctcggcggaccttccgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctggtcaagggcttttaccccagcgacattgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaagtgatgactcgag

SEQ ID NO: 5 - protein, 463 aa, Artificial Sequence
  RBD-human IgG1 Fc-fusion amino acid  sequence
EFKLATMGILPSPGMPALLSLVSLLSVLLMGCVAEASNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSGSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLE

SEQ ID NO: 6 - protein, 427 aa, Artificial Sequence
  RBD-human IgG1 Fc-fusion monomer amino acid sequence showing cleavage site
EASNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSGSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 729 bp, Artificial Sequence
  RBD monomer nucleotide sequence
gaattcaagcttgccaccatggggatacttcccagccctgggatgcctgcgctgctctccctcgtgagccttctctccgtgctgctgatgggttgcgtagctgaagctagcaatctgtgccccttcggcgaggtgttcaacgccacaagattcgcctctgtgtacgcctggaaccggaagcggatcagcaattgcgtggccgactacagcgtgctgtacaacagcgccagcttcagcaccttcaagtgctacggcgtgtcccctaccaagctgaacgacctgtgcttcaccaacgtgtacgccgacagcttcgtgatcagaggcgacgaagtgcggcagattgcccctggacagacaggcaagatcgccgattacaactacaagctgcccgacgacttcaccggctgtgtgattgcctggaacagcaacaacctggacagcaaagtcggcggcaactacaactacctgtaccggctgttccgcaagagcaacctgaagcctttcgagcgggacatcagcaccgaaatctaccaggccggcagcaccccttgcaatggcgtggaaggcttcaactgctacttcccactgcaaagctacggcttccagcctacaaacggcgtgggctaccagccttacagagtggtggtgctgagcttcgagctgctgcatgctcctgccacagtgtgtggacctggatcccaccaccaccatcaccactgatgactcgag

SEQ ID NO: 8 - protein, 241 aa, Artificial Sequence
  RBD monomer amino acid sequence
EFKLATMGILPSPGMPALLSLVSLLSVLLMGCVAEASNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSHHHHHHLE

SEQ ID NO: 9 - protein, 205 aa, Artificial Sequence
  RBD monomer amino acid sequence showing cleavage site
EASNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSHHHHHH

SEQ ID NO: 10 - protein, 197 aa, Artificial Sequence
  RBD monomer amino acid sequence without His-tag showing cleavage site
EASNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 11 - DNA, 1461 bp, Artificial Sequence
  RBD tandem dimer nucleotide sequence
gaattcaagcttgccaccatggggatacttcccagccctgggatgcctgcgctgctctccctcgtgagccttctctccgtgctgctgatgggttgcgtagctgaagctagcagagtgcagcccaccgagagcatcgtgcggttccccaacatcaccaatctgtgccctttcggcgaggtgttcaacgccaccagattcgcctctgtgtacgcctggaaccggaagcggatcagcaattgcgtggccgactacagcgtgctgtacaacagcgccagcttcagcaccttcaagtgctacggcgtgtcccctaccaagctgaacgacctgtgcttcaccaacgtgtacgccgacagcttcgtgatcagaggcgacgaagtgcggcagattgcccctggacagacaggcaagatcgccgattacaactacaagctgcccgacgacttcaccggctgtgtgattgcctggaacagcaacaacctggacagcaaagtcggcggcaactacaactacctgtaccggctgttccggaagtccaacctgaagcctttcgagcgggacatcagcaccgagatctatcaggccggcagcaccccttgcaatggcgtggaaggcttcaactgctacttcccactgcagtcctacggcttccagcctacaaacggcgtgggctaccagccttacagagtggtggtgctgagcttcgagctgctgcatgctcctgccacagtgtgcggccctaagaaaagcaccaacctggtcaagaacaagcgggtgcagccaacagagtccattgtgcgctttccgaatatcaccaacctctgtccattcggggaagtctttaatgccacacgcttcgccagcgtctacgcttggaataggaagagaatctccaactgcgtggcagattactccgtcctgtacaattccgccagcttttccacgtttaagtgttatggcgtcagccccacaaagctcaatgatctctgctttaccaatgtctatgccgactcctttgtgattcgcggagatgaagtccgccagatcgctccaggacagaccggaaaaatcgctgactataattacaagctcccggatgatttcacgggatgcgtgatcgcttggaactccaacaatctggattccaaagttggcgggaattacaattatctctatcgcctcttccgcaagagcaatctgaagccctttgagagagacatctctacagaaatctaccaggctggctctaccccatgcaacggcgtcgaggggtttaattgctatttcccgctgcaaagctacgggtttcagcccaccaatggcgtcggatatcagccctatcgcgtggtggtcctgtcttttgaactgctgcacgccccagctaccgtgtgtggcccaaaaaagtccaccaatctcgtgaagaacaaaggatcccaccaccaccatcaccactgatgactcgag

SEQ ID NO: 12 - protein, 485 aa, Artificial Sequence
  RBD tandem dimer amino acid sequence
EFKLATMGILPSPGMPALLSLVSLLSVLLMGCVAEASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSHHHHHHLE

SEQ ID NO: 13 - protein, 449 aa, Artificial Sequence
  RBD tandem dimer amino acid sequence showing cleavage site
EASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSHHHHHH

SEQ ID NO: 14 - protein, 441 aa, Artificial Sequence
  RBD tandem dimer amino acid sequence without His-tag showing cleavage site
EASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 15 - protein, 194 aa, Severe acute respiratory syndrome coronavirus 2
NLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 16 - protein, 221 aa, Mus musculus
CKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 17 - protein, 5 aa, Artificial Sequence
  Linker amino acid sequence
GSGSG

SEQ ID NO: 18 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 219 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 20 - protein, 219 aa, Severe acute respiratory syndrome coronavirus 2; identical to SEQ ID NO: 19

SEQ ID NO: 21 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - protein, 425 aa, Artificial Sequence
  Purified Beta RBD human IgG1 Fc-fusion monomer
SNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGSGSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 425 aa, Artificial Sequence
  RBD-human IgG1 Fc-fusion monomer amino acid sequence
SNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSGSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 203 aa, Artificial Sequence
  RBD monomer amino acid sequence
SNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSHHHHHH

SEQ ID NO: 25 - protein, 195 aa, Artificial Sequence
  Purified RBD monomer amino acid sequence without His-tag
SNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 26 - protein, 447 aa, Artificial Sequence
  RBD tandem dimer amino acid sequence
SRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSHHHHHH

SEQ ID NO: 27 - protein, 439 aa, Artificial Sequence
  RBD tandem dimer amino acid sequence without His-tag
SRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 28 - protein, 194 aa, Severe acute respiratory syndrome coronavirus 2
NLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGP

SEQ ID NO: 29 - DNA, 1344 bp, Artificial Sequence
  Beta RBD-human IgG1 Fc-fusion nucleotide sequence with leader sequence
atgcctgcgctgctctccctcgtgagccttctctccgtgctgctgatgggttgcgtagctgaagctagcaatctgtgccccttcggcgaggtgttcaacgccacaagattcgcctctgtgtacgcctggaaccggaagcggatcagcaattgcgtggccgactacagcgtgctgtacaacagcgccagcttcagcaccttcaagtgctacggcgtgtcccctaccaagctgaacgacctgtgcttcaccaacgtgtacgccgacagcttcgtgatcagaggcgacgaagtgcggcagattgcccctggacagacaggcaacatcgccgattacaactacaagctgcccgacgacttcaccggctgtgtgattgcctggaacagcaacaacctggacagcaaagtcggcggcaactacaactacctgtaccggctgttccgcaagagcaacctgaagcctttcgagcgggacatcagcaccgaaatctaccaggccggcagcaccccttgcaatggcgtgaaaggcttcaactgctacttcccactgcaaagctacggcttccagcctacatacggcgtgggctaccagccttacagagtggtggtgctgagcttcgagctgctgcatgctcctgccacagtgtgtggacctggatccggcagcggtacccacacctgtcctccatgtcctgctccagaactgctcggcggaccttccgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctggtcaagggcttttaccccagcgacattgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaagtga

SEQ ID NO: 30 - DNA, 1278 bp, Artificial Sequence
  Beta RBD-human IgG1 Fc-fusion nucleotide sequence without leader sequence
agcaatctgtgccccttcggcgaggtgttcaacgccacaagattcgcctctgtgtacgcctggaaccggaagcggatcagcaattgcgtggccgactacagcgtgctgtacaacagcgccagcttcagcaccttcaagtgctacggcgtgtcccctaccaagctgaacgacctgtgcttcaccaacgtgtacgccgacagcttcgtgatcagaggcgacgaagtgcggcagattgcccctggacagacaggcaacatcgccgattacaactacaagctgcccgacgacttcaccggctgtgtgattgcctggaacagcaacaacctggacagcaaagtcggcggcaactacaactacctgtaccggctgttccgcaagagcaacctgaagcctttcgagcgggacatcagcaccgaaatctaccaggccggcagcaccccttgcaatggcgtgaaaggcttcaactgctacttcccactgcaaagctacggcttccagcctacatacggcgtgggctaccagccttacagagtggtggtgctgagcttcgagctgctgcatgctcctgccacagtgtgtggacctggatccggcagcggtacccacacctgtcctccatgtcctgctccagaactgctcggcggaccttccgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctggtcaagggcttttaccccagcgacattgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaagtga

SEQ ID NO: 31 - DNA, 1344 bp, Artificial Sequence
  RBD-human IgG1 Fc-fusion nucleotide sequence with leader sequence
atgcctgcgctgctctccctcgtgagccttctctccgtgctgctgatgggttgcgtagctgaagctagcaatctgtgccccttcggcgaggtgttcaacgccacaagattcgcctctgtgtacgcctggaaccggaagcggatcagcaattgcgtggccgactacagcgtgctgtacaacagcgccagcttcagcaccttcaagtgctacggcgtgtcccctaccaagctgaacgacctgtgcttcaccaacgtgtacgccgacagcttcgtgatcagaggcgacgaagtgcggcagattgcccctggacagacaggcaagatcgccgattacaactacaagctgcccgacgacttcaccggctgtgtgattgcctggaacagcaacaacctggacagcaaagtcggcggcaactacaactacctgtaccggctgttccgcaagagcaacctgaagcctttcgagcgggacatcagcaccgaaatctaccaggccggcagcaccccttgcaatggcgtggaaggcttcaactgctacttcccactgcaaagctacggcttccagcctacaaacggcgtgggctaccagccttacagagtggtggtgctgagcttcgagctgctgcatgctcctgccacagtgtgtggacctggatccggcagcggtacccacacctgtcctccatgtcctgctccagaactgctcggcggaccttccgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctggtcaagggcttttaccccagcgacattgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaagtga

SEQ ID NO: 32 - DNA, 1278 bp, Artificial Sequence
  RBD-human IgG1 Fc-fusion nucleotide sequence without leader sequence
agcaatctgtgccccttcggcgaggtgttcaacgccacaagattcgcctctgtgtacgcctggaaccggaagcggatcagcaattgcgtggccgactacagcgtgctgtacaacagcgccagcttcagcaccttcaagtgctacggcgtgtcccctaccaagctgaacgacctgtgcttcaccaacgtgtacgccgacagcttcgtgatcagaggcgacgaagtgcggcagattgcccctggacagacaggcaagatcgccgattacaactacaagctgcccgacgacttcaccggctgtgtgattgcctggaacagcaacaacctggacagcaaagtcggcggcaactacaactacctgtaccggctgttccgcaagagcaacctgaagcctttcgagcgggacatcagcaccgaaatctaccaggccggcagcaccccttgcaatggcgtggaaggcttcaactgctacttcccactgcaaagctacggcttccagcctacaaacggcgtgggctaccagccttacagagtggtggtgctgagcttcgagctgctgcatgctcctgccacagtgtgtggacctggatccggcagcggtacccacacctgtcctccatgtcctgctccagaactgctcggcggaccttccgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctggtcaagggcttttaccccagcgacattgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaagtga

SEQ ID NO: 33 - protein, 455 aa, Artificial Sequence
  Beta RBD human IgG1 Fc-fusion monomer
MGILPSPGMPALLSLVSLLSVLLMGCVAEASNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGSGSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK